(19) United States
(12) Patent Application Publication
(10) Pub. No.: US 2026/0110034 A1
(43) Pub. Date: Apr. 23, 2026

(54) IMPROVED METHODS TO DIAGNOSE HEAD AND NECK CANCER AND USES THEREOF

(71) Applicant: The University of North Carolina at Chapel Hill, Chapel Hill, NC (US)

(72) Inventors: Wendell Gray Yarbrough, Chapel Hill, NC (US); Natalia Isaeva, Pittsboro, NC (US); Travis Parke Schrank, Chapel Hill, NC (US)

(21) Appl. No.: 18/534,101

(22) PCT Filed: Jun. 9, 2022
(86) PCT No.: PCT/US2022/032871

§ 371 Date: Dec. 8, 2023

Related U.S. Application Data
(60) Provisional application No. 63/208,547, filed on Jun. 9, 2021.

(51) Int. Cl. C12Q 1/6886; G01N 33/574

(52) U.S. Cl. CPC C12Q 1/6886; G01N 33/57557; C12Q 2600/106; C12Q 2600/118; C12Q 2600/156; C12Q 2600/158

(57) ABSTRACT

This disclosure provides a method for evaluating the prognosis of a head and neck cancer patient. The head and neck cancer may be human papillomavirus positive (HPV+) and originate in the upper aerodigestive tract (e.g. oropharynx, nasopharynx, nasal cavity, sinus, or hypopharynx). In addition, the disclosure provides a method for predicting a response of a head and neck cancer patient to a selected treatment. The disclosure also provides a method for generating an improved head and neck cancer biomarker signature for patient prognosis and uses thereof.

### CROSS REFERENCE TO RELATED APPLICATIONS

This application a § 371 U.S. National Stage of International Application PCT/US2022/032871, filed 9 Jun. 2022, having Atty. Docket No. 150-34-PCT, which claims the benefit of claims the benefit of 63/208,547 filed 9 Jun. 2021, Yarbrough et al., entitled IMPROVED METHODS TO DIAGNOSE HEAD AND NECK CANCER AND USES THEREOF, Atty. Dkt. No. 150-34-PROV which are hereby incorporated by reference in their entireties.

### STATEMENT REGARDING FEDERALLY SPONSORED RESEARCH OR DEVELOPMENT

This invention was made with government support under DC005360, DE029754, DE029241, and CA236762 awarded by the National Institutes of Health. The government has certain rights in the invention.

### REFERENCE TO A “SEQUENCE LISTING,” A TABLE, OR A COMPUTER PROGRAM LISTING APPENDIX SUBMITTED AS AN ASCII TEXT FILE

This application contains a sequence listing appendix. It has been submitted electronically via EFS-Web as an ASCII text file entitled 150-34-PCT_2022-06-09A_ST25.txt”. The sequence listing is 1639 bytes in size, and was created on Jun. 9, 2022. It is hereby incorporated by reference in its entirety.

### 1. FIELD

The present disclosure provides a method for evaluating the prognosis of a head and neck cancer patient. Specifically, human papilloma virus (HPV) positive, HPV+, squamous cell carcinomas of the oropharynx, oral cavity, hypopharynx, nasopharynx, and sinonasal cavity. In addition, the disclosure provides a method for predicting a response of a head and neck cancer patient to a selected treatment. The disclosure also provides a method for generating an improved head and neck cancer biomarker signature for patient prognosis and uses thereof.

### 2. BACKGROUND

### 2.1. Introduction

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present disclosure.

Head and neck cancers arise in mucosal epithelia lining various cavities in the head and neck region, such as the oral cavity, sinonasal cavity, larynx and throat. According to the American Cancer Society, head and neck cancer accounts for about 4% of all cancers in the United States. In 2020 approximately 65,000 people (48,000 men and 17,000 women) developed head and neck cancer and approximately 14,500 people died (10,760 men and 3,740 women). A substantial portion of head and neck cancers are associated with human papilloma virus (HPV); whereas the remainder are linked to other risk factors, such as tobacco use and alcohol consumption.

HPV associated head & neck squamous cell carcinoma (HPV+ HNSCC) has now surpassed cervical cancer in incidence, and is the most commonly diagnosed malignancy caused by HPV in the USA.¹ HPV+ HNSCC is clinically distinguished from tumors not associated with HPV by immunohistochemical staining that showed expression of p16INK4a (p16+). HPV+ HNSCC has an improved prognosis compared to HNSCC not associated with HPV, leading to a distinct staging system for these tumors.^{2,3} The combination of improved outcomes and significant and lifelong therapeutic toxicity has encouraged study de-intensified therapy for patients with HPV+ HNSCC in effort to limit morbidity while preserving favorable outcomes.⁴⁻⁷ Initial results of these studies are mixed, likely because of the inadequacy of current prognosticators that are limited to clinical stage and tobacco history. Implementation of de-escalated therapy is being hampered by inability to identify appropriate low risk patients^{8,4}. Therefore, it has become a key goal of the head and neck research community to develop accurate prognostic biomarkers which could assist physicians in choosing the intensity of treatment.

### 3. SUMMARY OF THE DISCLOSURE

The present disclosure provides a method for evaluating the prognosis of a human papilloma virus (HPV) associated head and neck cancer patient, comprising detecting defects in nucleic acids encoding genes, or their expression products, for at least five biomarkers selected from the group consisting of TRAF3, CYLD, TRAF2, MYD88, NFKBIA, TNFAIP3, TRAF6, BIRC2, BIRC3, and MAP3K14 in a sample from the patient, normalized against a reference set of nucleic acids encoding genes, or their expression products, in the sample, wherein defects in the nucleic acids or their expression products is indicative of prognosis, thereby evaluating the prognosis of the head and neck cancer patient.

In the method, the presence of defects in the nucleic acids encoding genes, or their expression products, for the biomarkers is indicative of a good prognosis. Alternatively, the absence of defects in the nucleic acids encoding genes, or their expression products, for the biomarkers is indicative of a poor prognosis. The defects may be mutations or copy number alterations such as missense mutations, nonsense mutations, frameshift mutations, insertions, and/or deletions. The defects in nucleic acids encoding genes, or their expression products, for the biomarkers may be detected by next generation sequencing (NGS), nucleic acid hybridization, quantitative RT-PCR, or immunohistochemistry (IHC), immunocytochemistry (ICC), or immunofluorescence (IF).

The method for evaluating the prognosis of a head and neck cancer patient may further comprise assessment of a medical history, a family history, a physical examination, an endoscopic examination, imaging, a biopsy result, or a combination thereof so as to develop a treatment strategy for the head and neck cancer patient. The nucleic acids encoding genes may be isolated from a fixed, paraffin-embedded sample, or from core biopsy tissue or fine needle aspirate cells (which may be fresh or frozen) from the patient.

This disclosure also provides a method for predicting a response of a human papilloma virus (HPV) associated head and neck cancer patient to a selected treatment, comprising detecting defects in nucleic acids encoding genes, or their expression products, for at least five biomarkers selected from the group consisting of TRAF3, CYLD, TRAF2, MYD88, NFKBIA, TNFAIP3, TRAF6, BIRC2, BIRC3, and MAP3K14 in a sample from the patient, normalized against a reference set of nucleic acids encoding genes, or their expression products, in the sample, wherein defects in the nucleic acids, or their expression products, is indicative of a positive treatment response, thereby predicting the response of the head and cancer patient to the treatment. The treatment may be radiation therapy, chemotherapy, immunotherapy, surgery, targeted therapy, or a combination thereof. The methods disclosed herein are well-suited for determining if a patient would be appropriate for a de-intensification of therapy to reduce side effects and morbidity.

The disclosure also provides a kit comprising at least five nucleic acid probes, wherein each of said probes specifically binds to one of five distinct biomarker nucleic acids or fragments thereof selected from the group consisting of TRAF3, CYLD, TRAF2, MYD88, NFKBIA, TNFAIP3, TRAF6, BIRC2, BIRC3, and MAP3K14.

In addition, the disclosure provides a method for generating an improved human papilloma virus (HPV) associated head and neck cancer gene expression signature for patient prognosis, the method comprising: (a) training a dataset using TRAF3 and CYLD genomic alteration (mutational or copy number loss) status to identify genes having mRNA expression data associated with NF-kB activity; (b) selecting 10 or more genes with the strongest differential expression found to be associated with NF-kB pathway genomic alteration to be part of a NF-kB activity classifier; and (c) using related mRNA expression levels for the 10 or more genes to generate the improved head and neck cancer gene expression signature for patient prognosis. In one embodiment, 25 or more genes with the strongest prognostic signal are selected. Alternatively, 50 or 75 or more genes with the strongest prognostic signal are selected.

The disclosure also provides a method for evaluating the prognosis of a human papilloma virus (HPV) associated head and neck cancer patient, comprising measuring mRNA expression of at least 10 of the top genes selected from the genes listed of in Table 1 in a sample comprising a cancer cell from the patient, normalized against the expression levels of all RNA transcripts in the sample or a reference set of mRNA expression levels, wherein the mRNA expression levels of the at least 10 genes are indicative of NF-kB activity, thereby evaluating the prognosis of the head and neck cancer patient. In one embodiment, the mRNA expression of 25 or more top genes are measured. Alternatively, the mRNA expression of 50 or more genes is measured.

In the methods above, the head and neck cancer may be an oropharyngeal squamous cell carcinoma (OPSCC), a nasopharyngeal squamous cell carcinoma, a squamous cell carcinomas of the nasal cavity or paranasal sinuses, a squamous cell carcinoma of the oral cavity, or a squamous cell carcinoma of the hypopharynx.

The methods above may further comprise assessment of a medical history, a family history, a physical examination, an endoscopic examination, imaging, a biopsy result, or a combination thereof so as to develop a treatment strategy for the head and neck cancer patient. The nucleic acids encoding genes may be isolated from a fixed, paraffin-embedded sample, or from core biopsy tissue or fine needle aspirate cells (which may be fresh or frozen) from the patient.

The disclosure also provides a kit comprising at least five nucleic acid probes, wherein each of said probes specifically binds to one of five distinct biomarker nucleic acids or fragments thereof selected from the group consisting of TRAF3, CYLD, TRAF2, MYD88, NFKBIA, TNFAIP3, TRAF6, BIRC2, BIRC3, and MAP3K14. In an alternative embodiment, the kit provides antibodies specific for the expression products, or proteins encoded by, TRAF3, CYLD, TRAF2, MYD88, NFKBIA, TNFAIP3, TRAF6, BIRC2, BIRC3, and MAP3K14.

### 4. BRIEF DESCRIPTION OF THE FIGURES

FIG. 1A-1C. Genomic Alterations in NF-kB Related Genes in HPV+ HNSCC and Survival Analysis in the UNC cohort of HPV-positive head and neck tumors. FIG. 1A. Waterfall plot of genomic alteration for the indicated NF-kB related genes. Row annotation—Percent of tumors with gene altered. DEL—copy loss (log 2 ratio<−0.75). AMP—copy number amplification (log 2 ratio>0.75). MISS—missense, or in frame indel. FS_STOP—nonsense, frameshift. Kaplan-Meier Analyses of Overall Survival (FIG. 1B) and Recurrence Free Survival (FIG. 1C) demonstrating improved survival for patients whose tumors harbored defects in this set of NF-kB regulators.

FIG. 2. Machine Learning Approach to Define Expression Signature and Biological Tumor Groups. This figure shows a schematic of how mutations in DNA coding for TRAF3 and CYLD were used to generate the RNA expression signature to classify tumors.

FIG. 3. RNA Expression Changes Associated with TRAF3/CYLD Alterations and Deletions. Normalized log 2 (read counts per million), color scaled by row. Columns—Tumor Samples, organized by unguided clustering. Rows—Top 100 genes by p-value differentially expressed between high-confidence NF-kB active and inactive tumors (see methods for details). Row annotation—Known NF-kB target genes curated from literature review. Column annotation details: Both TRAF3 and CYLD Alteration—Any one of missense, nonsense, frameshift, shallow deletion, deep deletion in both TRAF3 and CYLD, Shallow Deletion—Gistic copy-number score=−1, Deep Deletion—Gistic copy-number score=−2, Stop Gained—frameshift or nonsense mutation. Missense—missense or in frame indel. Stop/Deep Del. TRAF3 or CYLD—Any one of nonsense, frameshift, deep deletion in TRAF3 and/or CYLD.

FIG. 4. Gene Set Enrichment Analysis. All available genes after data filtering (see methods) were ranked according to signal-to-noise ratio when comparing the two groups of tumors. The MiSigDB Hallmark TNFA/NF-kB gene set was tested for enrichment. NF-kB High Activity—tumors were defined according to RNA based classifications (see methods), these were compared to all other tumors in the study cohort. NF-kB Pathway Alteration—Any missense, nonsense, frameshift, shallow deletion, deep deletion in TRAF3 and/or CYLD, these were compared to all other tumors in the study cohort. Lines—enrichment score values. Dashed Line—maximum achieved enrichment score (NF-kB high activity only). Vertical Hashes—rank positions of the test gene set (Hallmark NF-kB).

FIG. 5A-5D. Kaplan-Meier Analysis of Recurrence-free Survival (RFS) and Progression Free Interval (PFI) of HPV+ OPSCC Patients. Recurrence-free survival (RFS) data was available for 57 HPV-positive patients from TCGA HNSCC cohort, therefore 4 patients were excluded from the presented RFS analysis. All patients had available progression free interval (PFI) data. P-values represent log-rank test. HR—Hazard Ratio. NF-kB High Active—Highly NF-kB active tumors by RNA expression as defined according to the RNA based classifier (see methods), these were compared to all other tumors (NF-kB Inactive) in the study cohort. NF-kB Pway Alt—Any missense, nonsense, frameshift, shallow deletion, deep deletion in TRAF3 and/or CYLD, these were compared to all other tumors (NF-kB Pway WT) in the study cohort. FIG. 5A-5B. Kaplan-Meier Analysis of Recurrence-free survival (RFS) of HPV+ HNSCC patients. P-values represent log-rank test. FIG. 5C-5D. Kaplan-Meier Analysis of Progression Free Interval (PFI) of HPV+ HNSCC patients. P-values represent log-rank test. H HR—Hazard Ratio. NF-κB Active—Highly NF-κB active tumors by RNA expression as defined according to the RNA based classifier (see methods), these were compared to all other tumors (NF-κB Inactive) in the study cohort. TRAF3/CYLD Alt—Any missense, nonsense, frameshift, deep deletion in TRAF3 and/or CYLD, these were compared to all other tumors (TRAF3/CYLD WT) in the study cohort. See FIG. 12 for grossly similar recurrence-free survival results.

FIG. 6 shows a model for the etiology of HPV+ HNSCC with a timeline for a proposed alternative model of HPV carcinogenesis. Mutations in a panel of genes (TRAF3, CYLD, TRAF2, MYD88, NFKBIA, TNFAIP3, TRAF6, BIRC2, BIRC3, MAP3K 14) or mRNA expression profiles from a set of genes (see Table 1) are indicative of constitutive NF-kB activity and episomal HPV. Cancer cells fitting this profile are more sensitive to DNA damage, thus patients with this profile would be potential candidates for deintensified therapies. In the classical HPV-induced carcinogenesis, the HPV genes are integrated into the human genome. In this scenario, cells exhibit a type I interferon (IFN) response and the cancer cells are resistant to radiation damage. Patients with cancer cells harboring the integrated HPV (classical HPV infection) would be candidates for more aggressive therapies.

FIG. 7A-7C. Development of an NF-kB Activity Related RNA Expression Classifier. FIG. 7A. Heatmap of RNA Expression Changes Associated with TRAF3/CYLD Alterations and Deletions. Normalized log 2 (read counts per million), color scaled by row. Columns—Tumor Samples, organized by unguided clustering. Rows—Top 100 genes by p-value differentially expressed between high-confidence NF-κB active vs. inactive tumors (see methods for details). Row annotation—Known NF-κB target genes curated from literature review. Column Annotation Details: Track 1 (green)—RNA classifier (“NF-κB active”) based on nearest centroid. Track 2 (green brown)—RNA classifier (“NF-κB highly active”) based on minimal classifier score identified for TRAF3/CYLD nonsense or frameshift mutation bearing tumors. Track 3 (orange)—Tumor contains a frameshift, nonsense, or deep deletion in TRAF3 or CYLD. Track 4 (purple)—Tumor contains a frameshift or nonsense mutation in TRAF3. Track 5 (lavender)—Tumor contains a deep deletion in TRAF3. Track 6 (pink)—Tumor contains a shallow deletion in TRAF3. Track 7 (army green)—Tumor contains a frameshift or nonsense mutation in CYLD. Track 8 (lime green)—Tumor contains a missense mutation in CYLD. Track 9 (yellow)—Tumor contains a deep deletion in CYLD. Track 10 (mustard)—Tumor contains a shallow deletion in CYLD. Track 11 (dark brown)—Tumor contains any alteration in both TRAF3 and CYLD. Shallow Deletion—Gistic copy-number score=−1, Deep Deletion—Gistic copy-number score=−2, Stop Gained—frameshift or nonsense mutation. Missense—missense or in frame indel. Stop Deep Del.—Any one of nonsense, frameshift, or deep deletion. FIG. 7B. Auto-correlation of RNA Gene Set before and after the machine learning (ML) procedure. FIG. 7C. Classifier Performance of Gene Sets before and after ML improvement, with increasing (simulated) error of measurement. Performance determined by area under the receiver operating characteristic curve. ***P value<5*10\hat{ }-4, **P value<5*10\hat{ }-3.

FIG. 8A-8C. Characterization of the NF-κB Activity Classifier Genes with Weighted Gene Correlation Network Analysis (WGCNA). Only modules with more than 250 and less than 5000 genes were analyzed. FIG. 8A. Expression Dissimilarity matrix with clustering dendrogram. For clarity, a subset of 1500 genes are displayed. Warmer colors (red) represent higher degrees of dissimilarity. Row and Column Annotations—WGCNA gene expression modules, colors correspond to module name, as in panel C. FIG. 8B. Proportion of Genes by WGCNA module. NF-κB Classifier Gene Set—Gene set (50 genes) used in the NF-κB activity classifier. All genes—Genes analyzed by WGCNA but not included in the NF-κB activity classifier. P-value represent chi-squared test. ***-p-value<0.0001. FIG. 8C. Hypergeometric Enrichment Plot. Identified WGCNA modules were screened for enrichment in Hallmark Gene Sets from MiSigDB. Warmer colors represent lower adjusted p-value (q-value). Only results with q<0.05 were displayed. Percent of module genes in Hallmark gene set is represented by point size. Q-values represent hypergeometric enrichment as reported by the EnrichR R package.

FIG. 9A-9B. NF-κB Activity Classifier Correlates with Patient Outcomes and Viral Integration Status. FIG. 9A. Heatmap of HPV16 Viral Gene Expression for 61 HPV16+ OPSCC tumors included in the TCGA. Columns—tumors. Rows—HPV16 viral genes. Column Annotations: NF-κB activity RNA—nearest classifier score, higher values are more proximal to the NF-κB active centroid. E6E7/E2E5 Ratio—[E6 expression (raw counts)+E7 expression (raw counts)]/[E2 expression (raw counts)+E5 expression (raw counts)]. The columns are organized by this metric which is reported to strongly correlated with viral genomic integration. Integration Status—HPV viral integration status as determined by the ViFi pipeline. FIG. 9B. Box Plot comparing NF-κB activity in integrated and episomal tumor groups. Integration as assigned by ViFi. NF-κB activity—Raw NF-κB classifier scores as in FIG. 9A. **p<0.001.

FIG. 10A-10D. NF-κB Activity Classifier Gene Expression is Cohesive and Correlates with Patient Outcomes in an Independent Validation Cohort. FIG. 10A. Histogram of single-sample (ss) GSEA Scores for NF-κB activity classifier genes for each tumor in the validation cohort. Class Boundary—an empiric threshold based on the bimodal distribution of scores to assign (binary) NF-κB activity status. FIG. 10B. Kaplan-Meier Analysis of Recurrence Free Survival of HPV+ HNSCC. P-values represent log-rank test. HR—Hazard Ratio. NF-κB Active/Inactive-NF-κB active tumors by RNA expression as defined according to the ssGSEA scores for NF-κB activity classifier genes determined for each tumor as in FIG. 10A. FIG. 10C. Scatter plot of tumors based on gross RNA expression in principle component space, the top two principal components are displayed. Colors—NF-κB activity groups as in FIG. 10A. FIG. 10D. Box Plot of principle component values comparing NF-κB activity groups. P-values represent Wilcoxen Rank-sum test. **p-value<0.001, ***p-value<5*10\hat{ }-9. % Var.—Percentage of total variance explained by the individual principal component. Inset—Scatter plot of NFkB ssGSEA scores vs. PC3.

FIG. 11A-11D. Expression of CYLD (FIG. 11A), pp 65 (FIG. 11B) and GPDH in U2OS parental and CYLD CRISPR clones as determined by immunoblotting. FIG. 11C. Schematic representations of CYLD protein and schema of CYLD N300S and D618A mutant constructions. FIG. 11D. NF-κB reporter activity in U2OS parental, U2OS CYLD CRISPR (control) cells, or U2OS CYLD CRISPR cells transiently transfected with wild-type or mutant CYLD constructs. t-test was used to compare U2OS to other conditions. **—adjusted p-value (Bonferroni correction)<0.05.

FIG. 12A-12B. Kaplan Meier plots showing recurrence free survival (RFS). See methods and FIG. 5A-5B for details.

### 5. DETAILED DESCRIPTION OF THE DISCLOSURE

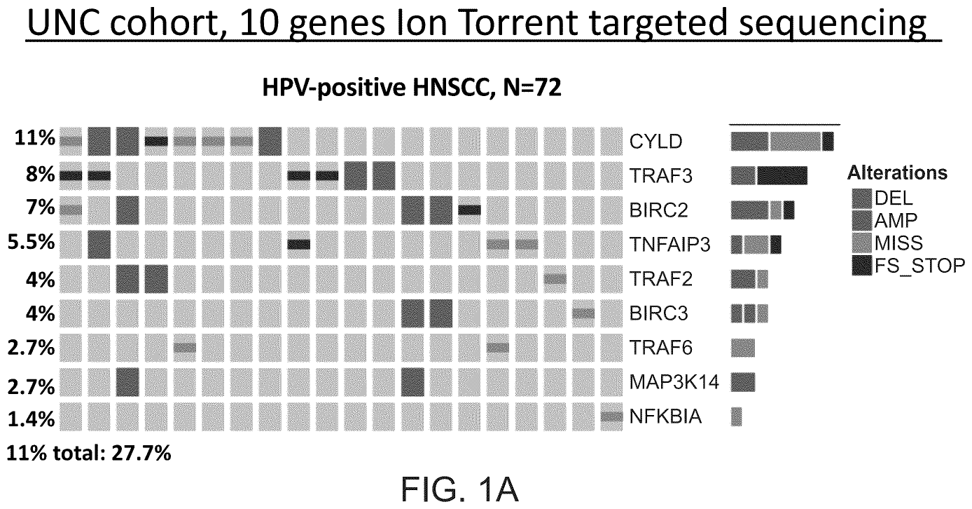

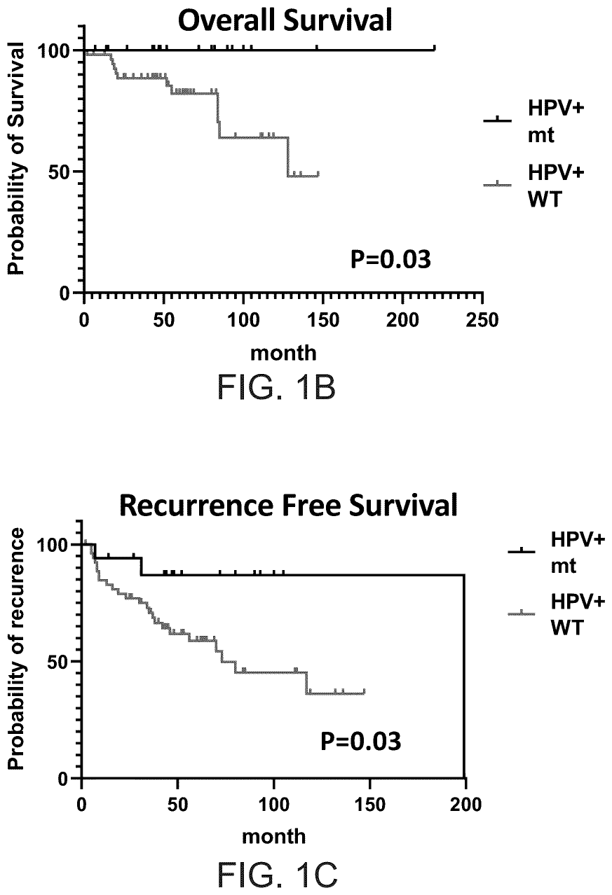

The literature has reported that mutations or copy number alterations in TRAF3 and CYLD genes correlated with improved outcomes in HPV+ HNSCC,^{6,9,10}. Given that these genes are regulators of the transcription factor NF-kB, gene defects altering a larger set of NF-kB regulatory genes (TRAF3, CYLD, TRAF2, MYD88, NFKBIA, TNFAIP3, TRAF6, BIRC2, BIRC3, MAP3K14), may improve prognostication. This 10 gene panel was tested and validated using a targeted sequencing strategy in a new cohort of patients. Results revealed that patients whose tumors lacked defects in NF-kB regulatory genes had significantly poorer overall survival (see FIG. 1A-1C).

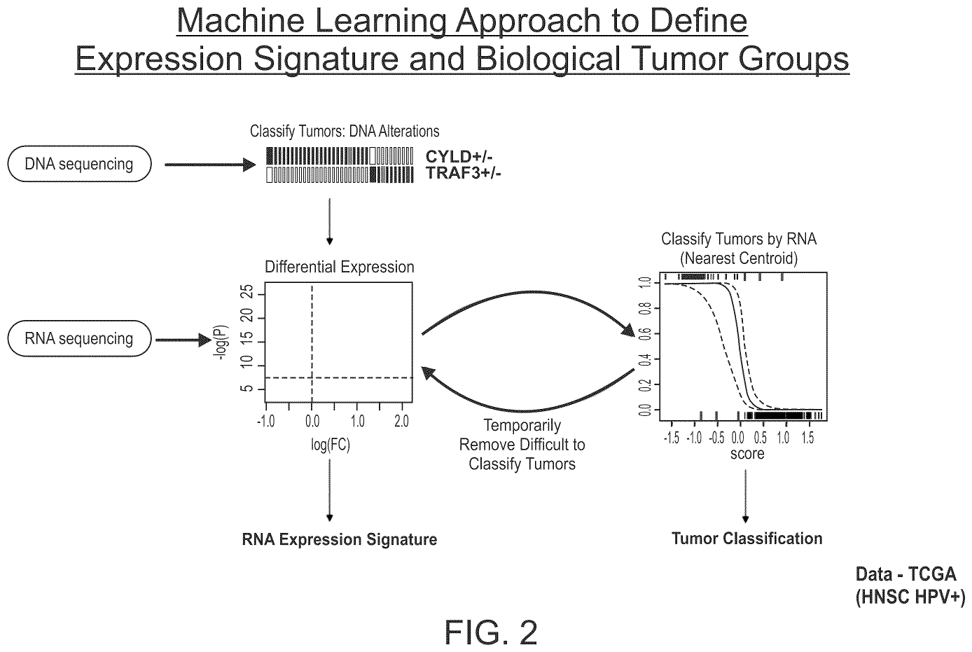

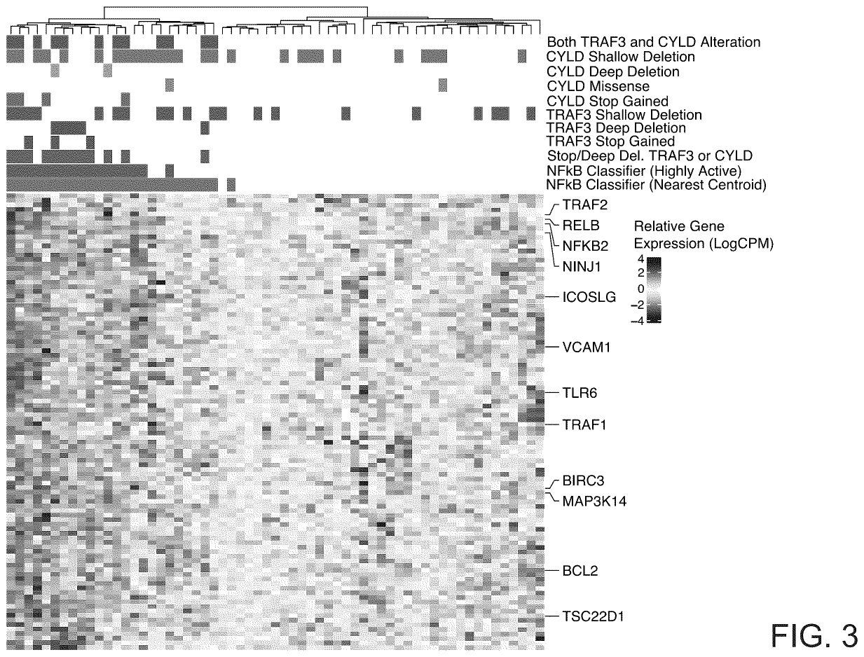

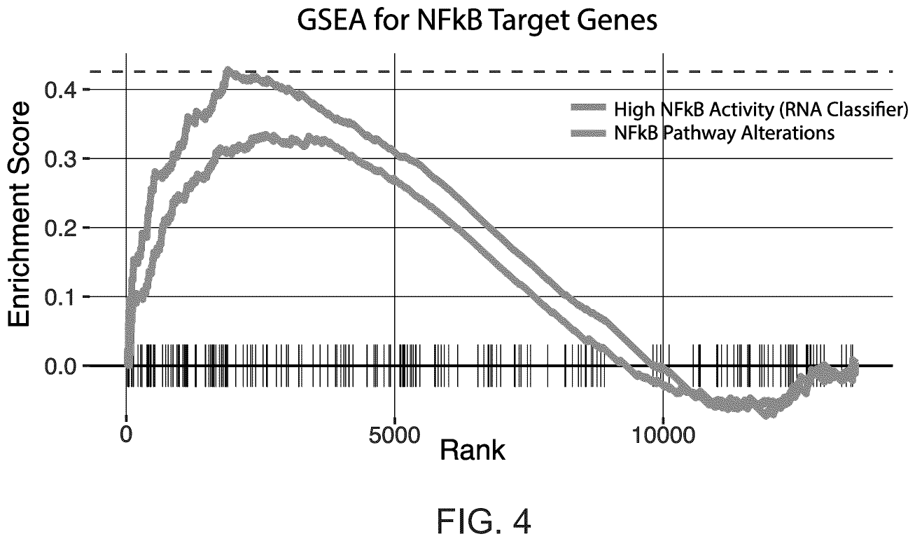

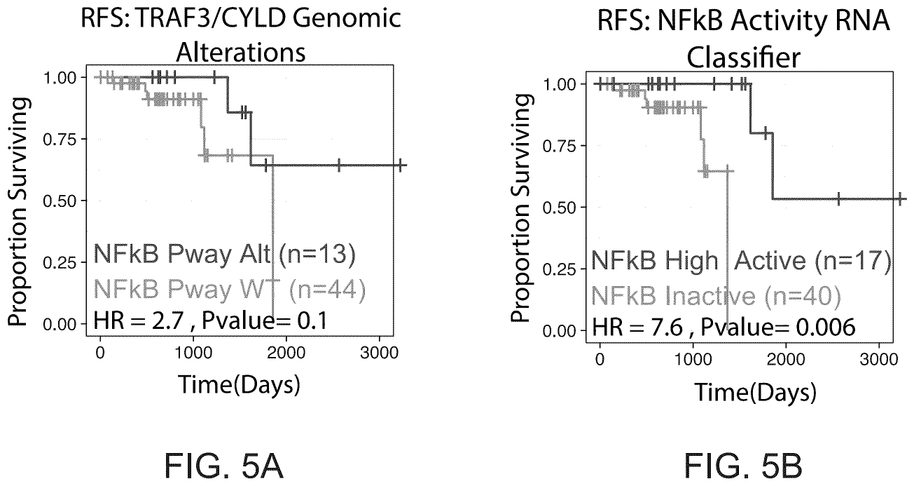

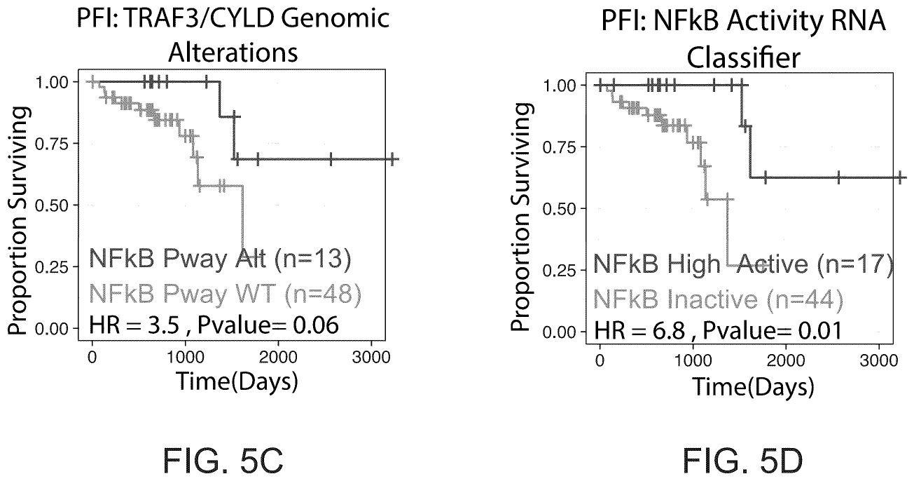

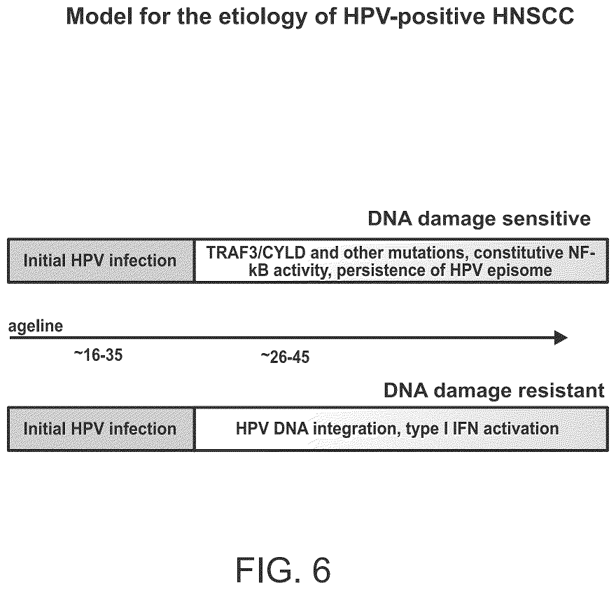

Since NF-kB is a transcription factor, gene expression levels may be different between tumors with and without mutations in NF-kB regulators. TRAF3/CYLD mutation status was used as a training set to identify an NF-kB related RNA expression classifier. FIG. 2 shows a general schematic for the method to use the DNA data (here TRAF3/CYLD mutation status) to classify tumors. These classified tumors were then used to generate an RNA expression signature for NF-kB regulators. The identified gene set is relevant to the disclosure, but also the above defined method by which the reference groups are defined. The genes listed are used to define a nearest centroid classifier. Using a proximity threshold to the NF-kB positive centroid defined by any deep deletion, frameshift, stop gain mutation in these genes gave the strongest prognostic signal. However, a simple nearest centroid was also predictive. These also strongly classify NF-kB related mutations and deletions with an unguided clustering approach (see FIG. 3). Using only high confidence class members to define the gene set of interest for subsequent classification, increased the NF-kB specificity of the genes (see FIG. 4). The classification approach also improved prediction of recurrence-free survival and progression free interval as compared to examining mutations and deletions alone (see FIG. 5A-5D). The classification strategy in addition to the gene set is an important innovation, as the ideal gene set may or may not vary according to the sequencing technology utilized, but the method to define predictive transcriptional classifiers starting with mutational data is likely to be highly generalizable.

The methods disclosed herein may be useful to select patients for treatment deintensification. Treatment deintensification may include reducing chemotherapy related toxicity by replacing cisplatin with an EGFR inhibitor, e.g., cetuximab (ERBITUX®); reducing the chemotherapy dose/duration; or elimination of chemotherapy. Alternatively, the deintensification may be the reduction of the radiotherapy dose regimen. For a review, see Kelly et al., (2016) Eur. J. Cancer November 68 125-133. Examples of targeted therapies with potential for HNSCC include a monoclonal antibody targeting the epidermal growth factor receptor (EGFR) extracellular domain such as Cetuximab, Panitumumab, Nimotuzumab, Zalutumumab, Sym004, ABBV-221; a small molecule targeting the EGFR tyrosine kinase such as Erlotinib, Gefitinib, Dacomitinib, or Afatinib; a small molecule targeting phosphoinositide 3-kinase (PI3K), Buparlisib, SF1126, Alpelisib, INCB050465, Copanlisib, or IPI-549; a small molecule targeting the mechanistic target of rapamycin (mTOR) such as Sirolimus, Everolimus, or Temsirolimus; a small molecule or oligonucleotide targeting signal transducer and activator of transcription 3 (STAT3) such as C188-9, Decoy, or AZD9150; or a monoclonal antibody targeting programmed cell death protein 1 (PD-1) or cytotoxic T-lymphocyte-associated protein (CTLA-4) such as Pembrolizumab, Nivolumab, or Ipilimumab. See Santuray (2018) Trends in Cancer 4 (5) 385-396 for a review.

In addition to HPV+ HNSCC, the methods disclosed herein may be useful for other cancers associated with activated NF-kB, such as EBV—associated nasopharyngeal cancer or HPV cancers where the HPV genome does not integrate in the DNA of the cancer cells. Non-integrating HPV is also known as episomal HPV. While the vast majority of HPV cervical cancers involve integration of the HPV into the genome of the host cell, the methods disclosed herein may be useful for the rare (3%) of cervical cancer cases that harbor NF-kB activating TRAF3/CYLD mutations.

In summary, this disclosure is directed to two related ways to assign NF-kB activation in HPV+ HNSCC, that is by identification of genetic defects in regulators of NF-kB and an RNA based classifier trained on mutational data. These tools may be readily translated to clinical practice. Furthermore, the improved mutational classifier has been validated in two distinct cohorts.

### 5.1. Definitions

While the following terms are believed to be well understood by one of ordinary skill in the art, the following definitions are set forth to facilitate explanation of the presently disclosed subject matter.

As used herein, “head and neck cancer” refers to cancer that arises in mucosal epithelia in the head or neck region, such as cancers in the nasal cavity, sinuses (e.g., paranasal sinuses), lips, mouth (e.g., oral cavity), salivary glands, throat (e.g., nasopharynx, oropharynx and hypopharynx), larynx, thyroid and parathyroids. An example of a head and neck cancer is a squamous cell carcinoma, such as oropharyngeal squamous cell carcinoma (OPSCC).

TRAF3 is Homo sapiens TNF receptor associated factor 3 (TRAF3), RefSeqGene (LRG_229) on chromosome 14, NCBI Reference Sequence: NG_027973.1 (CAP-1, CAP1, CD40 bp, CRAF1, IIAE5, LAP1, RNF118). CYLD is Homo sapiens CYLD lysine 63 deubiquitinase (CYLD), RefSeqGene (LRG_491) on chromosome 16, NCBI Reference Sequence: NG_012061.1 (also known as BRSS, CDMT, CYLD1, CYLDI, EAC, FTDALS8, MFT, MFT1, SBS, TEM, USPL2). TRAF2 is Homo sapiens TNF receptor associated factor 2 (TRAF2), mRNA, NCBI Reference Sequence: NM_021138.4 (also known as MGC: 45012, RNF117, TRAP, TRAP3). MYD88 is Homo sapiens MYD88 innate immune signal transduction adaptor (MYD88), RefSeqGene (LRG_157) on chromosome 3, NCBI Reference Sequence: NG_016964.1 (also known as IMD68, MYD88D). NFKBIA is Homo sapiens NFKB Inhibitor Alpha (NFKBIA) also known as IKBA, MAD-3, NFKBI, located on chromosome 14 NCBI reference sequence NG_007571.1. TNFAIP3 is Homo sapiens TNF alpha induced protein 3 (TNFAIP3), RefSeqGene on chromosome 6, NCBI Reference Sequence: NG_032761.1 (also known A20, AISBL, OTUD7C, TNFAIP2). TRAF6 is Homo sapiens TNF receptor associated factor 6 (TRAF6), transcript variant 2, mRNA, NCBI Reference Sequence: NM_004620.4 or Homo sapiens TNF receptor associated factor 6 (TRAF6), transcript variant 1, mRNA, NCBI Reference Sequence: NM_145803.3 (also known as MGC:3310, RNF85). BIRC2 is Homo sapiens baculoviral IAP repeat containing 2 (BIRC2), transcript variant 1, mRNA, NCBI Reference Sequence: NM_001166.5; Homo sapiens baculoviral IAP repeat containing 2 (BIRC2), transcript variant 2, mRNA, NCBI Reference Sequence: NM_001256163.1, or Homo sapiens baculoviral IAP repeat containing 2 (BIRC2), transcript variant 3, mRNA, NCBI Reference Sequence: NM_001256166.2 (also known as API1, HIAP2, Hiap-2, MIHB, RNF48, c-IAP1, cIAPI). BIRC3 is Homo sapiens baculoviral IAP repeat containing 3 (BIRC3), RefSeqGene on chromosome 11, NCBI Reference Sequence: NG_065365.1 (also known as AIP1, API2, CIAP2, HAIP1, HIAP1, IAP-1, MALT2, MIHC, RNF49, c-IAP2). MAP3K14 is Homo sapiens mitogen-activated protein kinase kinase kinase 14 (MAP3K14), RefSeqGene (LRG_1222) on chromosome 17, NCBI Reference Sequence: NG_033823.1 (also known as FTDCRIB, HS, HSNIK, NIK). ESR1 is Homo sapiens estrogen receptor 1 (ESR1), RefSeqGene (LRG_992) on chromosome 6, NCBI Reference Sequence: NG_008493.2 (also known as ER, ESR, ESRA, ESTRR, Era, NR3A1).

All genes names here refer to HUGO Gene Nomenclature Committee (genenames.org) reference gene names and include all transcript variants from the all associated genomic regions as defined by the HUGO gene nomenclature database.

As used herein, the term “reference set” may be an internal, external, or a universal reference set of nucleic acids or expression products used to calibrate a particular sample. For example, an internal reference set of nucleic acids may be obtained using normal tissue or a blood sample from the subject. Alternatively, an internal reference set may based on the total RNA in the sample. In another embodiment, the reference set may be a set of one or more housekeeping genes, e.g., human acidic ribosomal protein (HuPO), β-actin (BA), cyclophylin (CYC), glyceraldehyde-3-phosphate dehydrogenase (GAPDH), phosphoglycerokinase (PGK), β2-microglobulin (B2M), β-glucuronidase (GUS), hypoxanthine phosphoribosyltransferase (HPRT), transcription factor IID TATA binding protein (TBP), transferrin receptor (TfR), human acidic ribosomal protein (HuPO), elongation factor-1-α (EF-1-α), metastatic lymph node 51 (MLN51), or ubiquitin conjugating enzyme (UbcH5B). See Dheda et al. 2004 Bio Techniques 37:112-119. An external reference set may be obtained from clinical studies to determine normal ranges and ranges for head and neck cancer. Alternatively, the reference set may be based on a particular patient population such as smokers, gender or race. In yet another embodiment, the reference set may be a universal reference set. Many commercial vendors sell cDNA and RNA reference sets of genes or reference libraries.

Throughout the present specification, the terms “about” and/or “approximately” may be used in conjunction with numerical values and/or ranges. The term “about” is understood to mean those values near to a recited value. For example, “about 40 [units]” may mean within ±25% of 40 (e.g., from 30 to 50), within ±20%, ±15%, ±10%, ±9%, ±8%, ±7%, ±6%, ±5%, ±4%, ±3%, ±2%, ±1%, less than #1%, or any other value or range of values therein or there below. Alternatively, depending on the context, the term “about” may mean±one half a standard deviation, ±one standard deviation, or ±two standard deviations. Furthermore, the phrases “less than about [a value]” or “greater than about [a value]” should be understood in view of the definition of the term “about” provided herein. The terms “about” and “approximately” may be used interchangeably.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

As used herein, the verb “comprise” as used in this description and in the claims and its conjugations are used in its non-limiting sense to mean that items following the word are included, but items not specifically mentioned are not excluded.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. The present disclosure may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

### 5.2. Samples

The sample may be from a patient suspected of having head and neck cancer or from a patient diagnosed with head and neck cancer, e.g., for confirmation of diagnosis or establishing a clear margin or for the detection of head and neck cancer cells in other tissues such as lymph nodes, or circulating tumor cells. The biological sample may also be from a subject with an ambiguous diagnosis in order to clarify the diagnosis. The sample may be obtained for the purpose of differential diagnosis, e.g., a subject with a histopathologically benign lesion to confirm the diagnosis. The sample may also be obtained for the purpose of prognosis, i.e., determining the course of the disease and selecting primary treatment options. Tumor staging and grading are examples of prognosis. The sample may also be evaluated to select or monitor therapy, selecting likely responders in advance from non-responders or monitoring response in the course of therapy. In addition, the sample may be evaluated as part of post-treatment ongoing surveillance of patients who have had head and neck cancer.

Samples may be obtained using any of a number of methods in the art. Examples of biological samples comprising potential cancer cells include those obtained from excised skin biopsies, such as punch biopsies, shave biopsies, core needle biopsies, fine needle aspirates (FNA), or surgical excisions; or biopsy from non-cutaneous tissues such as lymph node tissue, mucosa, other embodiments. In addition, the sample may be from a distant metastatic site, a soft tissue, e.g., lung, liver, bone, skin, or brain. Representative biopsy techniques include, but are not limited to, excisional biopsy, incisional biopsy, pinch biopsy, forceps biopsy, needle biopsy, or surgical biopsy. An “excisional biopsy” refers to the removal of an entire tumor mass with a small margin of normal tissue surrounding it. An “incisional biopsy” refers to the removal of a wedge of tissue that includes a cross-sectional diameter of the tumor. A diagnosis or prognosis made by endoscopy or fluoroscopy may require a “core-needle biopsy” of the tumor mass, or a “fine-needle aspiration biopsy” which generally contains a suspension of cells from within the tumor mass. The biological sample may be a microdissected sample, such as a PALM-laser (Carl Zeiss MicroImaging GmbH, Germany) capture microdissected sample.

A sample may also be a sample of muscosal surfaces, blood and blood fractions or products (e.g., serum, plasma, platelets, red blood cells, white blood cells, circulating tumor cells isolated from blood, free DNA isolated from blood, and the like), sputum, saliva, lymph and tongue tissue, cultured cells, e.g., primary cultures, explants, and transformed cells, stool, urine, etc. The sample may also be vascular tissue or cells from blood vessels such as microdissected blood vessel cells of endothelial origin. A sample is typically obtained from a eukaryotic organism, most preferably a mammal such as a primate e.g., chimpanzee or human, cow, dog, cat; or a rodent, e.g., guinea pig, rat, mouse, rabbit.

A sample can be treated with a fixative such as formaldehyde and embedded in paraffin (FFPE) and sectioned for use in the methods of the invention. Alternatively, fresh or frozen tissue may be used. These cells may be fixed, e.g., in alcoholic solutions such as 100% ethanol or 3:1 methanol:acetic acid. Nuclei can also be extracted from thick sections of paraffin-embedded specimens to reduce truncation artifacts and eliminate extraneous embedded material. Typically, biological samples, once obtained, are harvested and processed prior to nucleic acid analysis using standard methods known in the art. Such processing typically includes protease treatment and additional fixation in an aldehyde solution such as formaldehyde.

### 5.2.1. Polynucleotide Sequence Amplification and Determination

In many instances, it is desirable to amplify a nucleic acid sequence using any of several nucleic acid amplification procedures which are well known in the art. Specifically, nucleic acid amplification is the chemical or enzymatic synthesis of nucleic acid copies which contain a sequence that is complementary to a nucleic acid sequence being amplified (template). The methods and kits of the invention may use any nucleic acid amplification or detection methods known to one skilled in the art, such as those described in U.S. Pat. No. 5,525,462 (Takarada et al.); U.S. Pat. No. 6,114,117 (Hepp et al.); U.S. Pat. No. 6,127,120 (Graham et al.); U.S. Pat. No. 6,344,317 (Urnovitz); U.S. Pat. No. 6,448,001 (Oku); U.S. Pat. No. 6,528,632 (Catanzariti et al.); and PCT Pub. No. WO 2005/111209 (Nakajima et al.); all of which are incorporated herein by reference in their entirety.

In some embodiments, the nucleic acids may be amplified by PCR amplification using methodologies known to one skilled in the art. One skilled in the art will recognize, however, that amplification can be accomplished by other known methods, such as ligase chain reaction (LCR), Qβ-replicase amplification, rolling circle amplification, transcription amplification, self-sustained sequence replication, nucleic acid sequence-based amplification (NASBA), each of which provides sufficient amplification. Branched-DNA technology may also be used to qualitatively demonstrate the presence of a sequence of the technology which may quantitatively determine the amount of this particular genomic sequence in a sample. Nolte reviews branched-DNA signal amplification for direct quantitation of nucleic acid sequences in clinical samples (Nolte, 1998, Adv. Clin. Chem. 33:201-235).

The PCR process is well known in the art and is thus not described in detail herein. For a review of PCR methods and protocols, see, e.g., Innis et al., eds., PCR Protocols, A Guide to Methods and Application, Academic Press, Inc., San Diego, Calif. 1990; U.S. Pat. No. 4,683,202 (Mullis); which are incorporated herein by reference in their entirety. PCR reagents and protocols are also available from commercial vendors, such as Roche Molecular Systems. PCR may be carried out as an automated process with a thermostable enzyme. In this process, the temperature of the reaction mixture is cycled through a denaturing region, a primer annealing region, and an extension reaction region automatically. Machines specifically adapted for this purpose are commercially available.

### 5.2.2. High Throughput and Single Molecule Sequencing Technology

Suitable next generation sequencing technologies are widely available. Examples include the 454 Life Sciences platform (Roche, Branford, CT) (Margulies et al. 2005 Nature, 437, 376-380); Illumina's Genome Analyzer, Illumina's MiSeq System, Illumina's NextSeq System, Illumina's MiniSeq System, (Illumina, San Diego, CA; Bibkova et al., 2006, Genome Res. 16, 383-393; U.S. Pat. Nos. 6,306,597 and 7,598,035 (Macevicz); U.S. Pat. No. 7,232,656 (Balasubramanian et al.)); or DNA Sequencing by Ligation, SOLID System (Applied Biosystems/Life Technologies; U.S. Pat. Nos. 6,797,470, 7,083,917, 7,166,434, 7,320,865, 7,332,285, 7,364,858, and 7,429,453 (Barany et al.); or the Helicos True Single Molecule DNA sequencing technology (Harris et al., 2008 Science, 320, 106-109; U.S. Pat. Nos. 7,037,687 and 7,645,596 (Williams et al.); 7,169,560 (Lapidus et al.); 7,769,400 (Harris)), the single molecule, real-time (SMRT™) technology of Pacific Biosciences, and sequencing (Soni and Meller, 2007, Clin. Chem. 53, 1996-2001) which are incorporated herein by reference in their entirety. These systems allow the sequencing of many nucleic acid molecules isolated from a specimen at high orders of multiplexing in a parallel fashion (Dear, 2003, Brief Funct. Genomic Proteomic, 1 (4), 397-416 and McCaughan and Dear, 2010, J. Pathol., 220, 297-306). Each of these platforms allow sequencing of clonally expanded or non-amplified single molecules of nucleic acid fragments. Certain platforms involve, for example, (i) sequencing by ligation of dye-modified probes (including cyclic ligation and cleavage), (ii) pyrosequencing, (iii) targeted next-generation sequencing from bisulfite treated DNA and (iv) single-molecule sequencing.

Pyrosequencing is a nucleic acid sequencing method based on sequencing by synthesis, which relies on detection of a pyrophosphate released on nucleotide incorporation. Generally, sequencing by synthesis involves synthesizing, one nucleotide at a time, a DNA strand complimentary to the strand whose sequence is being sought. Study nucleic acids may be immobilized to a solid support, hybridized with a sequencing primer, incubated with DNA polymerase, ATP sulfurylase, luciferase, apyrase, adenosine 5′ phosphsulfate and luciferin. Nucleotide solutions are sequentially added and removed. Correct incorporation of a nucleotide releases a pyrophosphate, which interacts with ATP sulfurylase and produces ATP in the presence of adenosine 5′ phosphosulfate, fueling the luciferin reaction, which produces a chemiluminescent signal allowing sequence determination. Machines for pyrosequencing are available from Qiagen, Inc. (Valencia, CA). An example of a system that can be used by a person of ordinary skill based on pyrosequencing generally involves the following steps: ligating an adaptor nucleic acid to a study nucleic acid and hybridizing the study nucleic acid to a bead; amplifying a nucleotide sequence in the study nucleic acid in an emulsion; sorting beads using a picoliter multiwell solid support; and sequencing amplified nucleotide sequences by pyrosequencing methodology (e.g., Nakano et al., 2003, J. Biotech. 102, 117-124). Such a system can be used to exponentially amplify amplification products generated by a process described herein, e.g., by ligating a heterologous nucleic acid to the first amplification product generated by a process described herein.

Next-generation sequencing (NGS) is a nucleic acid sequencing method based on sequencing by synthesis, where fluorescently labeled deoxyribonucleotide triphosphates (dNTPs) catalyzed by DNA polymerase are incorporated into a DNA temple through cycles of DNA synthesis and nucleotides are identified by fluorophore excitation at each incorporation step. NGS allows this process to take place in a multiplex reaction across millions of DNA fragments in parallel. Generally, sequencing by synthesis involves synthesizing, one nucleotide at a time, a DNA strand complimentary to the strand whose sequence is being sought. Study nucleic acids may be immobilized to a solid support, hybridized with a sequencing primer, and incubated with DNA polymerase in the presence of fluorescently labeled dNTPS. After each cycle, the image is scanned and the emission wavelength and intensity are recorded and used to identify the base incorporated. This process is repeated multiple times to create a specific read length of bases.

Certain single-molecule sequencing embodiments are based on the principal of sequencing by synthesis, and utilize single-pair Fluorescence Resonance Energy Transfer (single pair FRET) as a mechanism by which photons are emitted as a result of successful nucleotide incorporation. The emitted photons often are detected using intensified or high sensitivity cooled charge-couple-devices in conjunction with total internal reflection microscopy (TIRM). Photons are only emitted when the introduced reaction solution contains the correct nucleotide for incorporation into the growing nucleic acid chain that is synthesized as a result of the sequencing process. In FRET based single-molecule sequencing or detection, energy is transferred between two fluorescent dyes, sometimes polymethine cyanine dyes Cy3 and Cy5, through long-range dipole interactions. The donor is excited at its specific excitation wavelength and the excited state energy is transferred, non-radiatively to the acceptor dye, which in turn becomes excited. The acceptor dye eventually returns to the ground state by radiative emission of a photon. The two dyes used in the energy transfer process represent the “single pair”, in single pair FRET. Cy3 often is used as the donor fluorophore and often is incorporated as the first labeled nucleotide. Cy5 often is used as the acceptor fluorophore and is used as the nucleotide label for successive nucleotide additions after incorporation of a first Cy3 labeled nucleotide. The fluorophores generally are within 10 nanometers of each other for energy transfer to occur successfully.

An example of a system that can be used based on single-molecule sequencing generally involves hybridizing a primer to a study nucleic acid to generate a complex; associating the complex with a solid phase; iteratively extending the primer by a nucleotide tagged with a fluorescent molecule; and capturing an image of fluorescence resonance energy transfer signals after each iteration (e.g., Braslavsky et al., PNAS 100(7): 3960-3964 (2003); U.S. Pat. No. 7,297,518 (Quake et al.) which are incorporated herein by reference in their entirety). Such a system can be used to directly sequence amplification products generated by processes described herein. In some embodiments, the released linear amplification product can be hybridized to a primer that contains sequences complementary to immobilized capture sequences present on a solid support, a bead or glass slide for example. Hybridization of the primer-released linear amplification product complexes with the immobilized capture sequences, immobilizes released linear amplification products to solid supports for single pair FRET based sequencing by synthesis. The primer often is fluorescent, so that an initial reference image of the surface of the slide with immobilized nucleic acids can be generated. The initial reference image is useful for determining locations at which true nucleotide incorporation is occurring. Fluorescence signals detected in array locations not initially identified in the “primer only” reference image are discarded as non-specific fluorescence. Following immobilization of the primer-released linear amplification product complexes, the bound nucleic acids often are sequenced in parallel by the iterative steps of, a) polymerase extension in the presence of one fluorescently labeled nucleotide, b) detection of fluorescence using appropriate microscopy, TIRM for example, c) removal of fluorescent nucleotide, and d) return to step a with a different fluorescently labeled nucleotide.

The technology described herein may be practiced with digital PCR. Digital PCR was developed by Kalinina and colleagues (Kalinina et al., 1997, Nucleic Acids Res. 25; 1999-2004) and further developed by Vogelstein and Kinzler (1999, Proc. Natl. Acad. Sci. U.S.A. 96; 9236-9241). The application of digital PCR is described by Cantor et al. (PCT Pub. Nos. WO 2005/023091A2 (Cantor et al.); WO 2007/092473 A2, (Quake et al.)), which are hereby incorporated by reference in their entirety. Digital PCR takes advantage of nucleic acid (DNA, cDNA or RNA) amplification on a single molecule level, and offers a highly sensitive method for quantifying low copy number nucleic acid. Fluidigm® Corporation offers systems for the digital analysis of nucleic acids.

In some embodiments, nucleotide sequencing may be by solid phase single nucleotide sequencing methods and processes. Solid phase single nucleotide sequencing methods involve contacting sample nucleic acid and solid support under conditions in which a single molecule of sample nucleic acid hybridizes to a single molecule of a solid support. Such conditions can include providing the solid support molecules and a single molecule of sample nucleic acid in a “microreactor.” Such conditions also can include providing a mixture in which the sample nucleic acid molecule can hybridize to solid phase nucleic acid on the solid support. Single nucleotide sequencing methods useful in the embodiments described herein are described in PCT Pub. No. WO 2009/091934 (Cantor).

In certain embodiments, nanopore sequencing detection methods include (a) contacting a nucleic acid for sequencing (“base nucleic acid,” e.g., linked probe molecule) with sequence-specific detectors, under conditions in which the detectors specifically hybridize to substantially complementary subsequences of the base nucleic acid; (b) detecting signals from the detectors and (c) determining the sequence of the base nucleic acid according to the signals detected. In certain embodiments, the detectors hybridized to the base nucleic acid are disassociated from the base nucleic acid (e.g., sequentially dissociated) when the detectors interfere with a nanopore structure as the base nucleic acid passes through a pore, and the detectors disassociated from the base sequence are detected.

A detector also may include one or more regions of nucleotides that do not hybridize to the base nucleic acid. In some embodiments, a detector is a molecular beacon. A detector often comprises one or more detectable labels independently selected from those described herein. Each detectable label can be detected by any convenient detection process capable of detecting a signal generated by each label (e.g., magnetic, electric, chemical, optical and the like). For example, a CD camera can be used to detect signals from one or more distinguishable quantum dots linked to a detector.

The invention encompasses methods known in the art for enhancing the sensitivity of the detectable signal in such assays, including, but not limited to, the use of cyclic probe technology (Bakkaoui et al., 1996, Bio Techniques 20:240-8, which is incorporated herein by reference in its entirety); and the use of branched probes (Urdea et al., 1993, Clin. Chem. 39, 725-6; which is incorporated herein by reference in its entirety). The hybridization complexes are detected according to well-known techniques in the art.

Reverse transcribed or amplified nucleic acids may be modified nucleic acids. Modified nucleic acids can include nucleotide analogs, and in certain embodiments include a detectable label and/or a capture agent. Examples of detectable labels include, without limitation, fluorophores, radioisotopes, colorimetric agents, light emitting agents, chemiluminescent agents, light scattering agents, enzymes and the like. Examples of capture agents include, without limitation, an agent from a binding pair selected from antibody/antigen, antibody/antibody, antibody/antibody fragment, antibody/antibody receptor, antibody/protein A or protein G, hapten/anti-hapten, biotin/avidin, biotin/streptavidin, folic acid/folate binding protein, vitamin B12/intrinsic factor, chemical reactive group/complementary chemical reactive group (e.g., sulfhydryl/maleimide, sulfhydryl/haloacetyl derivative, amine/isotriocyanate, amine/succinimidyl ester, and amine/sulfonyl halides) pairs, and the like. Modified nucleic acids having a capture agent can be immobilized to a solid support in certain embodiments.

Next generation sequencing techniques may be applied to measure expression levels or count numbers of transcripts using RNA-seq or whole transcriptome shotgun sequencing. See, e.g., Mortazavi et al. 2008 Nat Meth 5(7) 621-627 or Wang et al. 2009 Nat Rev Genet 10(1) 57-63. Nucleic acids in the invention may be counted using methods known in the art. In one embodiment, NanoString's nCounter® system may be used (Seattle, WA). Geiss et al. 2008 Nat Biotech 26(3) 317-325; U.S. Pat. No. 7,473,767 (Dimitrov). In addition, NanoString's Digital Spatial Profiling (DSP) platform may be used for nucleic acid or protein detection. Blank et al., 2018 Nature Medicine 24 1655-1661; Amaria et al., 2018 Nature Medicine 24 1649-1654. Alternatively, Fluidigm's Dynamic Array system may be used (South San Francisco, CA). Byrne et al. 2009 PLOS ONE 4 e7118; Helzer et al. 2009 Can Res 69 7860-7866. For reviews, see also Zhao et al. 2011 Sci China Chem 54(8) 1185-1201 and Ozsolak and Milos 2011 Nat Rev Genet 12 87-98.

### 5.3. Classifiers and Classifier Methods

Pattern recognition (PR) methods have been used widely to characterize many different types of problems ranging from linguistics, fingerprinting, chemistry to psychology. In the context of the methods described herein, pattern recognition is the use of multivariate statistics, both parametric and non-parametric, to analyze data, and hence to classify samples and to predict the value of some dependent variable based on a range of observed measurements. There are two main approaches. One set of methods is termed “unsupervised” and these simply reduce data complexity in a rational way and also produce display plots that can be interpreted by the human eye. The other approach is termed “supervised” whereby a training set of samples with known class or outcome is used to produce a mathematical model and which is then evaluated with independent validation data sets.

Unsupervised PR methods are used to analyze data without reference to any other independent knowledge. Examples of unsupervised pattern recognition methods include principal component analysis (PCA), hierarchical cluster analysis (HCA), and non-linear mapping (NLM).

Alternatively, it has proved efficient to use a “supervised” approach to data analysis. Here, a “training set” of biomarker expression data is used to construct a statistical model that predicts correctly the “class” of each sample. This training set is then tested with independent data (referred to as a test or validation set) to determine the robustness of the computer-based model. These models are sometimes termed “expert systems,” but may be based on a range of different mathematical procedures. Supervised methods can use a data set with reduced dimensionality (for example, the first few principal components), but typically use unreduced data, with all dimensionality. In all cases the methods allow the quantitative description of the multivariate boundaries that characterize and separate each class, for example, each class of cancer in terms of its biomarker expression profile. It is also possible to obtain confidence limits on any predictions, for example, a level of probability to be placed on the goodness of fit (see, for example, Sharaf; Illman; Kowalski, eds. (1986). Chemometrics. New York: Wiley). The robustness of the predictive models can also be checked using cross-validation, by leaving out selected samples from the analysis.

Examples of supervised pattern recognition methods include the following: artificial neural networks (ANN) (see, for example, Wasserman (1993). Advanced methods in neural computing. John Wiley & Sons, Inc; O'Hare & Jennings (Eds.). (1996). Foundations of distributed artificial intelligence (Vol. 9). Wiley); Bayesian methods (see, for example, Bretthorst (1990). An introduction to parameter estimation using Bayesian probability theory. In Maximum entropy and Bayesian methods (pp. 53-79). Springer Netherlands; Bretthorst, G. L. (1988). Bayesian spectrum analysis and parameter estimation (Vol. 48). New York: Springer-Verlag); consensus clustering (see, for example, Senbabaoglu et al., 2014 “Critical limitations of consensus clustering in class discovery” Sci Reports 4:6207, pp 1-13); K-nearest neighbor analysis (KNN) (see, for example, Brown and Martin 1996 J Chem Info Computer Sci 36(3): 572-584); linear discriminant analysis (LDA) (see, for example, Nillson (1965). Learning machines. New York); nearest centroid methods (Dabney 2005 Bioinformatics 21(22): 4148-4154 and Tibshirani et al. 2002 Proc. Natl. Acad. Sci. USA 99(10): 6576-6572); partial least squares analysis (PLS) (see, for example, Wold (1966) Multivariate analysis 1:391-420; Joreskog (1982) Causality, structure, prediction 1:263-270); probabilistic neural networks (PNNs) (see, for example, Bishop & Nasrabadi (2006). Pattern recognition and machine learning (Vol. 1, p. 740). New York: Springer; Specht, (1990). Probabilistic neural networks. Neural networks, 3(1), 109-118); rule induction (RI) (see, for example, Quinlan (1986) Machine learning, 1(1), 81-106); soft independent modeling of class analysis (SIMCA) (see, for example, Wold, (1977) Chemometrics: theory and application 52:243-282); support vector machines (SVM) (see, for example Noble (2006) “What is a support vector machine?” Computational Biology 24 (12) 1565-1567); and unsupervised hierarchical clustering (see for example Herrero 2001 Bioinformatics 17(2) 126-136).

It is often useful to pre-process data, for example, by addressing missing data, translation, scaling, weighting, etc. Multivariate projection methods, such as principal component analysis (PCA) and partial least squares analysis (PLS), are so-called scaling sensitive methods. By using prior knowledge and experience about the type of data studied, the quality of the data prior to multivariate modeling can be enhanced by scaling and/or weighting. Adequate scaling and/or weighting can reveal important and interesting variation hidden within the data, and therefore make subsequent multivariate modeling more efficient. Scaling and weighting may be used to place the data in the correct metric, based on knowledge and experience of the studied system, and therefore reveal patterns already inherently present in the data.

### 5.4. Compositions and Kits

The invention provides compositions and kits detecting the biomarkers described herein using antibodies or other reagents specific for the nucleic acids specific for the polynucleotides. Kits for carrying out the diagnostic assays of the invention typically include, in suitable container means, (i) a probe that comprises an antibody or nucleic acid sequence that specifically binds to the marker polynucleotides of the invention, (ii) a label for detecting the presence of the probe and (iii) instructions for how to measure the level the polynucleotide. The kits may include several antibodies or polynucleotide sequences encoding biomarkers disclosed herein, e.g., a first antibody and/or second and/or third and/or additional antibodies that recognize the biomarkers or specific nucleic acids. In one embodiment the nucleic acids in the kit are the forward and reverse PCR primers for the biomarkers disclosed herein. The container means of the kits will generally include at least one vial, test tube, flask, bottle, syringe and/or other container into which a first antibody specific for one of the polypeptides or a first nucleic acid specific for one of the polynucleotides of the present invention may be placed and/or suitably aliquoted. Where a second and/or third and/or additional component is provided, the kit will also generally contain a second, third and/or other additional container into which this component may be placed. Alternatively, a container may contain a mixture of more than one antibody or nucleic acid reagent, each reagent specifically binding a different marker in accordance with the present invention. The kits of the present invention will also typically include means for containing the antibody or nucleic acid probes in close confinement for commercial sale. Such containers may include injection and/or blow-molded plastic containers into which the desired vials are retained.

The kits may further comprise positive and negative controls, as well as instructions for the use of kit components contained therein, in accordance with the methods of the present invention.

### 5.5. Computing Devices

A computing device may be implemented in programmable hardware devices such as processors, digital signal processors, central processing units, field programmable gate arrays, programmable array logic, programmable logic devices, cloud processing systems, or the like. The computing devices may also be implemented in software for execution by various types of processors. An identified device may include executable code and may, for instance, comprise one or more physical or logical blocks of computer instructions, which may, for instance, be organized as an object, procedure, function, or other construct. Nevertheless, the executable of an identified device need not be physically located together but may comprise disparate instructions stored in different locations which, when joined logically together, comprise the computing device and achieve the stated purpose of the computing device. In another example, a computing device may be a server or other computer located within a hospital or out-patient environment and communicatively connected to other computing devices (e.g., POS equipment or computers) for managing accounting, purchase transactions, and other processes within the hospital or out-patient environment. In another example, a computing device may be a mobile computing device such as, for example, but not limited to, a smart phone, a cell phone, a pager, a personal digital assistant (PDA), a mobile computer with a smart phone client, or the like. In another example, a computing device may be any type of wearable computer, such as a computer with a head-mounted display (HMD), or a smart watch or some other wearable smart device. Some of the computer sensing may be part of the fabric of the clothes the user is wearing. A computing device can also include any type of conventional computer, for example, a laptop computer or a tablet computer. A typical mobile computing device is a wireless data access-enabled device (e.g., an iPHONE® smart phone, a BLACKBERRY® smart phone, a NEXUS ONE™ smart phone, an iPAD® device, smart watch, or the like) that is capable of sending and receiving data in a wireless manner using protocols like the Internet Protocol, or IP, and the wireless application protocol, or WAP. This allows users to access information via wireless devices, such as smart watches, smart phones, mobile phones, pagers, two-way radios, communicators, and the like. Wireless data access is supported by many wireless networks, including, but not limited to, Bluetooth, Near Field Communication, CDPD, CDMA, GSM, PDC, PHS, TDMA, FLEX, ReFLEX, iDEN, TETRA, DECT, DataTAC, Mobitex, EDGE and other 2G, 3G, 4G, 5G, and LTE technologies, and it operates with many handheld device operating systems, such as PalmOS, EPOC, Windows CE, FLEXOS, OS/9, JavaOS, iOS and Android. Typically, these devices use graphical displays and can access the Internet (or other communications network) on so-called mini- or micro-browsers, which are web browsers with small file sizes that can accommodate the reduced memory constraints of wireless networks. In a representative embodiment, the mobile device is a cellular telephone or smart phone or smart watch that operates over GPRS (General Packet Radio Services), which is a data technology for GSM networks or operates over Near Field Communication e.g. Bluetooth. In addition to a conventional voice communication, a given mobile device can communicate with another such device via many different types of message transfer techniques, including Bluetooth, Near Field Communication, SMS (short message service), enhanced SMS (EMS), multi-media message (MMS), email WAP, paging, or other known or later-developed wireless data formats. Although many of the examples provided herein are implemented on smart phones, the examples may similarly be implemented on any suitable computing device, such as a computer.

An executable code of a computing device may be a single instruction, or many instructions, and may even be distributed over several different code segments, among different applications, and across several memory devices. Similarly, operational data may be identified and illustrated herein within the computing device, and may be embodied in any suitable form and organized within any suitable type of data structure. The operational data may be collected as a single data set, or may be distributed over different locations including over different storage devices, and may exist, at least partially, as electronic signals on a system or network.

The described features, structures, or characteristics may be combined in any suitable manner in one or more embodiments. In the following description, numerous specific details are provided, to provide a thorough understanding of embodiments of the disclosed subject matter. One skilled in the relevant art will recognize, however, that the disclosed subject matter can be practiced without one or more of the specific details, or with other methods, components, materials, etc. In other instances, well-known structures, materials, or operations are not shown or described in detail to avoid obscuring aspects of the disclosed subject matter.

As used herein, the term “memory” is generally a storage device of a computing device. Examples include, but are not limited to, read-only memory (ROM) and random access memory (RAM).

The device or system for performing one or more operations on a memory of a computing device may be a software, hardware, firmware, or combination of these. The device or the system is further intended to include or otherwise cover all software or computer programs capable of performing the various heretofore-disclosed determinations, calculations, or the like for the disclosed purposes. For example, exemplary embodiments are intended to cover all software or computer programs capable of enabling processors to implement the disclosed processes. Exemplary embodiments are also intended to cover any and all currently known, related art or later developed non-transitory recording or storage mediums (such as a CD-ROM, DVD-ROM, hard drive, RAM, ROM, floppy disc, magnetic tape cassette, etc.) that record or store such software or computer programs. Exemplary embodiments are further intended to cover such software, computer programs, systems and/or processes provided through any other currently known, related art, or later developed medium (such as transitory mediums, carrier waves, etc.), usable for implementing the exemplary operations disclosed below.

In accordance with the exemplary embodiments, the disclosed computer programs can be executed in many exemplary ways, such as an application that is resident in the memory of a device or as a hosted application that is being executed on a server and communicating with the device application or browser via a number of standard protocols, such as TCP/IP, HTTP, XML, SOAP, REST, JSON and other sufficient protocols. The disclosed computer programs can be written in exemplary programming languages that execute from memory on the device or from a hosted server, such as BASIC, COBOL, C, C++, Java, Pascal, or scripting languages such as JavaScript, Python, Ruby, PHP, Perl, or other suitable programming languages.

As referred to herein, the terms “computing device” and “entities” should be broadly construed and should be understood to be interchangeable. They may include any type of computing device, for example, a server, a desktop computer, a laptop computer, a smart phone, a cell phone, a pager, a personal digital assistant (PDA, e.g., with GPRS NIC), a mobile computer with a smartphone client, or the like.

As referred to herein, a user interface is generally a system by which users interact with a computing device. A user interface can include an input for allowing users to manipulate a computing device, and can include an output for allowing the system to present information and/or data, indicate the effects of the user's manipulation, etc. An example of a user interface on a computing device (e.g., a mobile device) includes a graphical user interface (GUI) that allows users to interact with programs in more ways than typing. A GUI typically can offer display objects, and visual indicators, as opposed to text-based interfaces, typed command labels or text navigation to represent information and actions available to a user. For example, an interface can be a display window or display object, which is selectable by a user of a mobile device for interaction. A user interface can include an input for allowing users to manipulate a computing device, and can include an output for allowing the computing device to present information and/or data, indicate the effects of the user's manipulation, etc. An example of a user interface on a computing device includes a graphical user interface (GUI) that allows users to interact with programs or applications in more ways than typing. A GUI typically can offer display objects, and visual indicators, as opposed to text-based interfaces, typed command labels or text navigation to represent information and actions available to a user. For example, a user interface can be a display window or display object, which is selectable by a user of a computing device for interaction. The display object can be displayed on a display screen of a computing device and can be selected by and interacted with by a user using the user interface. In an example, the display of the computing device can be a touch screen, which can display the display icon. The user can depress the area of the display screen where the display icon is displayed for selecting the display icon. In another example, the user can use any other suitable user interface of a computing device, such as a keypad, to select the display icon or display object. For example, the user can use a track ball or arrow keys for moving a cursor to highlight and select the display object.

The display object can be displayed on a display screen of a mobile device and can be selected by and interacted with by a user using the interface. In an example, the display of the mobile device can be a touch screen, which can display the display icon. The user can depress the area of the display screen at which the display icon is displayed for selecting the display icon. In another example, the user can use any other suitable interface of a mobile device, such as a keypad, to select the display icon or display object. For example, the user can use a track ball or times program instructions thereon for causing a processor to carry out aspects of the present disclosure.

As referred to herein, a computer network may be any group of computing systems, devices, or equipment that are linked together. Examples include, but are not limited to, local area networks (LANs) and wide area networks (WANs). A network may be categorized based on its design model, topology, or architecture. In an example, a network may be characterized as having a hierarchical internetworking model, which divides the network into three layers: access layer, distribution layer, and core layer. The access layer focuses on connecting client nodes, such as workstations to the network. The distribution layer manages routing, filtering, and quality-of-server (QoS) policies. The core layer can provide high-speed, highly-redundant forwarding services to move packets between distribution layer devices in different regions of the network. The core layer typically includes multiple routers and switches.

The present subject matter may be a system, a method, and/or a computer program product. The computer program product may include a computer readable storage medium (or media) having computer readable program instructions thereon for causing a processor to carry out aspects of the present subject matter.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a RAM, a ROM, an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network, or Near Field Communication. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present subject matter may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++, Javascript or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present subject matter.

Aspects of the present subject matter are described herein with reference to flowchart illustrations and/or block diagrams of methods, apparatus (systems), and computer program products according to embodiments of the subject matter. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present subject matter. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Unless defined otherwise, all technical and scientific terms used herein have the same meanings as commonly understood by one of ordinary skill in the art to which this disclosure belongs. Preferred methods, devices, and materials are described, although any methods and materials similar or equivalent to those described herein can be used in the practice or testing of the present disclosure. All references cited herein are incorporated by reference in their entirety.

The following Examples further illustrate the disclosure and are not intended to limit the scope. In particular, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

### 6. EXAMPLE 1

### 6.1. Methods

### 6.1.1. Targeted Sequencing:

Primers were designed for exon capture Ion Torrent next-generation sequencing of tumor and matched normal tissues. All exons from 10 genes (TRAF3, CYLD, TRAF2, MYD88, NFKBIA, TNFAIP3, TRAF6, BIRC2, BIRC3, MAP3K14) were included in the primer panel that consists of 250 overlapping amplicons in a 2 primer pool format with overall coverage of 93.65%. DNA was extracted from paraffin embedded tumor and surrounding normal tissue using QIAamp DNA FFPE Tissue Kit and from corresponding blood samples using DNeasy Blood & Tissue Kit, these and the primer panel were provided to Mako Genomics for NGS. The sequencing was performed using an IonTorrent S5 sequencer and automated library prep station.

### 6.1.2. Mutational and Copy Number Calling:

For analysis of institutional cohort genomic data, single nucleotide polymorphisms and indels were called using Varscan2 with default settings, as well as minimum coverage depth of 25, minimum variant reads of 4 and minimum variant allele frequency of 0.05 for a call to be made. For copy number calling, reads per exon were assigned with the R processCounts package. Reads per exon were summed per gene and reads per gene (or exon) were found to be linearly correlated between tumor and normal samples. Reads per gene were normalized to total reads per sample and compared with normal using a test of measured proportions (prop.test( ) R function). Multiple comparison corrections were assigned using R project fdrTool. Log 2Ratio tumor/normal were calculated for data visualization. A log ratio>|0.75|(>0.75 or <−0.75) was empirically set at the limit of biological significance.

### 6.1.3. Data Acquisition:

De-identified, publicly available clinical and genomic data were utilized for this study. Clinical data for the TCGA head and neck squamous cohort was acquired through the Broad Firehose portal (gdac.broadinstitute.org) and UCSC Xena (xena.ucse.edu). Supplemental survival metrics (PFI) were acquired from Liu et al.¹¹ Per-gene quantified mRNA read count data, as well as per-gene discretized Gistic2 copy-number analysis data for TCGA-HNSC were downloaded from the Broad Firehose Portal. Variant calls were downloaded using the R TCGAbiolinks¹² package, calls performed with VarScan¹³ were used for all analyses.

### 6.1.4. Cohort Selection and Inclusion Criteria:

RNA assigned HPV status from the Firehose clinical annotations were used to assign HPV status, only HPV positive tumors were included. Tumors with TP53 mutations or deep deletions were excluded from the analysis. Anatomic subsites from the oropharynx, tonsil, base of tongue were included; nearby subsites of the hypopharynx and oral tongue were also included. Tumors from more distal sites (eg. Larynx, alveolar ridge, maxilla) were excluded. A total of 61 patients were found meeting these criteria.

### 6.1.5. Bioinformatics:

RNA read count data was preprocessed by filtering low expression genes so that the distribution of log₂cpm values as approximately Gaussian. Filtered read count data were then normalized using the trimmed means of M values methods provided in the R edgeR package.¹⁴ The Limma-voom pipeline was used for all subsequent differential expression analysis.¹⁵ All classifiers used the nearest centroid method, and were defined and cross validated using the R cancerclass package.¹⁶

To construct a high-performance RNA based classifier for NF-kB activity in HPV+ HNSCC, we employed a centroid classifier, trained on high confidence class members. Preliminary groups of NF-kB active and inactive tumors were assigned by mutational status, i.e., all tumors with deep deletions (Gistic −2) mutations (missense, nonsense, frame shift) in the NF-kB regulator genes TRAF3 and CYLD were considered to be NF-kB active, and other tumors inactive. An initial differential expression was performed between these preliminary groups, and a classifier defined based on the top 150 genes ranked by p-value. High confidence class members were defined as having correct initial assignment and having RNA expression values very similar to the class-defining average of expression (centroid). High confidence class members were then used for differential expression and construction of a final classifier. The top 50 genes (by p-value) were selected based on lack of improvement in the receiver operator characteristic with the addition of more genes. This final classifier had perfect performance on leave one out cross validation. Inclusion of the top 10 of 150 genes (See Table 1) in the final classifier, had similar performance to that using the top 50 genes. In one embodiment, the top 10 genes by p-value are selected. Alternatively, the top 20, top 30, top 40, top 50, top 75, or top 100 genes may be used. One skilled in the art could recognize that different subsets of classifiers using as few as 10 genes selected from the 150 genes listed in Table 1 may yield similar results. This is possible because of the robust transcriptomic differences identified related to NF-kB activation in HPV+ HNSCC. Alternatively, a selected group of 15, 20, 25, 30, 35, 40, 45, 50, 55, or more genes from Table 1 may be used. Furthermore, gene sets derived from other statistical methods such as count based differential expression or correlation analysis with the goal of defining genes that have variable expression according to genomic variant status of the specific genes discussed in paragraph above, are expected to yield similar prognostic information, even if the specific genes are not included in the list provided in Table 1. Although this disclosure primarily investigated a centroid based classification strategy, other classification strategies (consensus clustering, support vector machine) (see section 5.3 above for additional strategies) are also expected to yield similar results.

The all tumors in the selected cohort were then classified according to this final model using the nearest centroid method, for correlation with clinical and genomic data. For additional classifications of highly active NF-kB tumors, an empiric threshold was set for NF-kB activity at the distance of the frameshift or nonsense TRAF3/CYLD mutation farthest from the NF-kB active centroid.

### Survival Analysis:

RFS survival data was available for 57 of these patients (UCSC Xena). Both event status and times to events were very similar for PFI data extracted from Liu et al., although an atypical metric for survival in HPV+ OPSCC, the dataset provided values for all of the patients included in our study. We therefore, also present PFI data to demonstrate both the generalizability of our findings across multiple outcome metrics and also to validate the RFS related findings (n=57) with the full cohort (n=61). Survival statistics were generated with the R survival package (v3.2−7), and visualized with the R survminer package (0.4.8). p-values represent log-rank test.

### 6.1.6. Gene Set Enrichment Analysis:

Ranked gene lists were created using the signal to noise ratio for the change in expression between two groups of interest as defined in the popular GSEA software package distributed by the Broad Institute.^{17,18} Hallmark signatures from the MiSigDB were used as gene sets of interest.¹⁹ GSEA testing and related multiple comparison testing were performed with the R fgsea package.²⁰

### 7. REFERENCES (PART 1)

1. Pan C, Issaeva N, Yarbrough W G. HPV-driven oropharyngeal cancer: current knowledge of molecular biology and mechanisms of carcinogenesis. Cancers Head Neck. 2018; 3. doi:10.1186/s41199-018-0039-3
2. Doescher J, Veit J A, Hoffmann T K. [The 8th edition of the AJCC Cancer Staging Manual: Updates in otorhinolaryngology, head and neck surgery]. HNO. 2017; 65(12):956-961. doi:10.1007/s00106-017-0391-3
3. Zhan K Y, Eskander A, Kang S Y, et al. Appraisal of the AJCC 8th edition pathologic staging modifications for HPV-positive oropharyngeal cancer, a study of the National Cancer Data Base. Oral Oncol. 2017; 73:152-159. doi:10.1016/j.oraloncology.2017.08.020
4. Cheraghlou S, Yu P K, Otremba M D, et al. Treatment deintensification in human papillomavirus-positive oropharynx cancer: Outcomes from the National Cancer Data Base. Cancer. 2018; 124(4):717-726. doi:10.1002/cncr.31104
5. Chera B S, Amdur R J, Tepper J E, et al. Mature results of a prospective study of deintensified chemoradiotherapy for low-risk human papillomavirus-associated oropharyngeal squamous cell carcinoma. Cancer. 2018; 124(11):2347-2354. doi:10.1002/cncr.31338
6. Chera B S, Kumar S, Beaty B T, et al. Rapid Clearance Profile of Plasma Circulating Tumor HPV Type 16 DNA during Chemoradiotherapy Correlates with Disease Control in HPV-Associated Oropharyngeal Cancer. Clin Cancer Res Off J Am Assoc Cancer Res. 2019; 25(15):4682-4690. doi:10.1158/1078-0432.CCR-19-0211
7. Marur S, Li S, Cmelak A J, et al. E1308: Phase II Trial of Induction Chemotherapy Followed by Reduced-Dose Radiation and Weekly Cetuximab in Patients With HPV-Associated Resectable Squamous Cell Carcinoma of the Oropharynx-ECOG-ACRIN Cancer Research Group. J Clin Oncol Off J Am Soc Clin Oncol. 2017; 35(5):490-497. doi:10.1200/JCO.2016.68.3300
8 Pearlstein K A, Wang K, Amdur R J, et al. Quality of Life for Patients With Favorable-Risk HPV-Associated Oropharyngeal Cancer After De-intensified Chemoradiotherapy. Int J Radiat Oncol Biol Phys. 2019; 103(3):646-653. doi:10.1016/j.ijrobp.2018.10.033
9. Hajek M, Sewell A, Kaech S, Burtness B, Yarbrough W G, Issaeva N. TRAF3/CYLD mutations identify a distinct subset of human papillomavirus-associated head and neck squamous cell carcinoma. Cancer. 2017; 123(10):1778-1790. doi:10.1002/cncr.30570
10. Chera B S, Kumar S, Shen C, et al. Plasma Circulating Tumor HPV DNA for the Surveillance of Cancer Recurrence in HPV-Associated Oropharyngeal Cancer. J Clin Oncol Off J Am Soc Clin Oncol. 2020; 38(10):1050-1058. doi:10.1200/JCO.19.02444
11. Liu J, Lichtenberg T, Hoadley K A, et al. An Integrated TCGA Pan-Cancer Clinical Data Resource to Drive High-Quality Survival Outcome Analytics. Cell. 2018; 173(2):400-416.e11. doi:10.1016/j.cell.2018.02.052
12. Mounir M, Lucchetta M, Silva T C, et al. New functionalities in the TCGAbiolinks package for the study and integration of cancer data from GDC and GTEx. PLOS Comput Biol. 2019; 15(3):e1006701. doi:10.1371/journal.pcbi.1006701
13. Koboldt D C, Zhang Q, Larson D E, et al. VarScan 2: somatic mutation and copy number alteration discovery in cancer by exome sequencing. Genome Res. 2012; 22(3):568-576. doi:10.1101/gr.129684.111
14. Robinson M D, McCarthy D J, Smyth G K. edgeR: a Bioconductor package for differential expression analysis of digital gene expression data. Bioinforma Oxf Engl. 2010; 26(1):139-140. doi:10.1093/bioinformatics/btp616
15. Law C W, Chen Y, Shi W, Smyth G K. voom: precision weights unlock linear model analysis tools for RNA-seq read counts. Genome Biol. 2014; 15(2):R29. doi:10.1186/gb-2014-15-2-r29
16. Jan B, Kosztyla D, Törne C von, et al. cancerclass: An R Package for Development and Validation of Diagnostic Tests from High-Dimensional Molecular Data. J Stat Softw. 2014; 59(1):1-19. doi:10.18637/jss.v059.101
17. Subramanian A, Tamayo P, Mootha V K, et al. Gene set enrichment analysis: a knowledge-based approach for interpreting genome-wide expression profiles. Proc Natl Acad Sci USA. 2005; 102(43):15545-15550. doi:10.1073/pnas.0506580102
18. Mootha V K, Lindgren C M, Eriksson K-F, et al. PGC-1 alpha-responsive genes involved in oxidative phosphorylation are coordinately downregulated in human diabetes. Nat Genet. 2003; 34(3):267-273. doi:10.1038/ng1180
19. Liberzon A, Birger C, Thorvaldsdóttir H, Ghandi M, Mesirov J P, Tamayo P. The Molecular Signatures Database (MSigDB) hallmark gene set collection. Cell Syst. 2015; 1(6):417-425. doi:10.1016/j.cels.2015.12.004
20. Fast gene set enrichment analysis|bioRxiv. Accessed Oct. 29, 2020. https://www.biorxiv.org/content/10.1101/060012v2

### 8. EXAMPLE 2

### 8.1. Summary Example 2

Evolving understanding of head and neck squamous cell carcinoma (HNSCC) is leading to more specific diagnostic disease classifications. Among HNSCC caused by the human papilloma virus (HPV), tumors harboring defects in TRAF3 or CYLD) are associated with improved clinical outcomes and maintenance of episomal HPV. TRAF3 and CYLD are negative regulators of NF-κB and inactivating mutations of either leads to NF-κB overactivity. Activation of NF-κB is described in virally associated nasopharyngeal cancer caused by Epstein-Barr virus. Here, we developed and validated a gene expression classifier separating HPV+ HNSCCs based on NF-κB activity. As expected, the novel classifier is strongly enriched in NF-κB targets leading us to name it the NF-κB Activity Classifier (NAC). High NF-κB activity correlated with improved survival in two independent cohorts. Using NAC, tumors with high NF-κB activity but lacking defects in TRAF3 or CYLD were identified; thus, while TRAF3 or CYLD gene defects account for the majority of NF-κB activation in these tumors, unknown mechanisms also exist. The NAC correctly classified the functional consequences of two novel CYLD missense mutations. Using a reporter assay, we tested these CYLD mutations revealing that their activity to inhibit NF-kB was equivalent to the wild-type protein. Future applications of the NF-κB Activity Classifier may be to identify HPV+ HNSCC patients with better or worse survival with implications for treatment strategies.

### 8.2. Example 2

### 8.2.1. Introduction

Head and neck squamous cell carcinoma (HNSCC) is a devastating disease that impairs fundamental tissues involved in respiration, phonation, and digestion. It is categorized into two discrete diseases based on etiology: human papillomavirus (HPV) negative HNSCC, which is primarily caused by exposure to ethanol and tobacco, and HPV-associated (HPV+) HNSCC.(1) These forms of HNSCC have contrasting clinical, epidemiological, and histological features (2-4) with HPV+ HNSCC occurring in a younger population with less or no smoking history.(5, 6) HPV-mediated carcinogenesis occurs primarily in the reticulated epithelia of the oropharynx (e.g., tonsils, base of tongue) whereas HPV-negative HNSCC is found at all subsites (e.g., oral cavity, larynx).(2) Unfortunately, the global incidence of HPV+ HNSCC is increasing, and for nearly a decade, HPV has caused more head and neck cancers than uterine cervical cancers annually in the United States.(7, 8)

Since HPV+ HNSCC is a relatively new phenomenon (9), management of HNSCC has been driven by escalating therapies to improve cancer control in the more treatment-resistant HPV-negative HNSCC.(2, 6) While oncologic outcomes for HPV+ HNSCC are generally favorable, application of treatment paradigms developed for HPV-negative disease burdens many survivors of HPV+ HNSCC with lifelong debilitating treatment-associated side effects. (10) On the other hand, ˜30% of HPV+ HNSCC patients exhibit a more aggressive disease course and suffer recurrence. (11, 12) As such, there is a growing clinical demand to develop robust stratification tools to accurately identify patients with good or poor prognosis and that could be used to personalize treatment.

Attempts to identify survival phenotypes have leveraged underlying genomic distinctions. (3, 13) In particular, somatic defects in the NF-κB inhibitors TRAF3 and CYLD are found in ˜30% of HPV+ HNSCC tumors.(1, 13, 14) These gene defects are uncommon in uterine cervical cancer and HPV-negative HNSCC. While frequent TRAF3 or CYLD) inactivating mutations are found in B cell lymphomas, where constitutive NF-κB activity is known to play a key survival role,(15-17) these mutations are rarely found in solid tumors.(13) Exceptions with more frequent TRAF3 and CYLD mutations include two virally-associated cancers, HPV+ HNSCC and Epstein-Barr virus-associated nasopharyngeal carcinoma (NPC).(18-20) While initial studies focused on NF-κB activity as a defense against viral infections, further investigation revealed more nuance with some viruses, like EBV and HIV, depending on NF-κB activity to support viral replication and viral gene expression.(21-24) Given the frequency of TRAF3 and CYLD mutations and their correlation with HPV episomes, it is likely that HPV also exploits NF-κB activity during head and neck carcinogenesis.

The power of multi-variable models and/or multi-omic approaches can be harnessed to improve tumor subtyping.(25-28) For example, an RNA expression-based PARP inhibitor outcome prediction model in ovarian cancer outperformed BRCA1/2 mutational status in predicting treatment response.(27) In the present study, transcriptional differences between tumors with and without TRAI 3 and CYLD defects formed the basis for a novel classification of HPV+ HNSCC. Based on established roles of TRAF3 and CYLD as inhibitors of NF-κB, it was expected that the resultant classifier would segregate tumors on the basis of NF-κB activity. Gene set enrichment analysis confirmed that the classifier identified tumors with high or low NF-κB activity and, relative to TRAF3 and CYLD defects, this NF-κB Activity Classifier (NAC) improved identification of tumors with good and poor survival. Among TCGA specimens, two novel missense mutations in CYLD were identified: N300S and D618A.(13) To understand the implications of these point mutations, we used the NAC and correlated results with a cell-based assay to evaluate their effect on NF-κB transcriptional activity; our data show that both CYLD mutants are able to inhibit NF-κB similarly to wild-type CYLD.

Together, these studies provide a foundation for exploring treatment personalization using a pathway-centric RNA based classifier that identifies HPV+ HNSCC patients with good or poor prognosis and provides further insight into how loss of TRAF3 and CYLD activity supports HPV carcinogenesis in the head and neck.

### 8.3. MATERIALS and METHODS

### 8.3.1. Data Acquisition

Only de-identified, publicly available clinical and genomic data were utilized for this study. Per-gene quantified mRNA read count data, as well as per-gene discretized Gistic2 copy-number analysis data for the Cancer Genome Atlas (29) HNSCC, were downloaded from the Broad Firehose Portal (30). In this work, we consider a Gistic score of −2 synonymous with deep deletion, and Gistic score of −1 synonymous with a shallow deletion. Gistic uses a dynamic segmentation algorithm to define chromosomal arm level (−1) and deeper focal deletions (−2) based on per tumor thresholds (31). Clinical data for the TCGA HNSCC cohort were acquired from Liu et al.(32) Variant calls were downloaded using the R TCGAbiolinks (33) package; calls performed with VarScan (34) were used for all analyses. TCGA RNA sequencing BAM files were downloaded from dbGaP, with NIH request #99293-1 for project #27853: “Prognostic signature in head and neck cancer” (PI-N.I.).

### 8.3.2. Cohort Selection and Inclusion Criteria

RNA assigned HPV status from the Firehose clinical annotations were used to assign HPV status, only HPV positive tumors were included (35). Tumors with TP53 mutations or deep deletions were excluded from the analysis. Anatomic subsites from the oropharynx, tonsil, and base of tongue were included, and nearby subsites of the hypopharynx and oral tongue considering HPV+ TP53 wild-type tumors were likely an oropharyngeal primary. Tumors from more distal sites (e.g., larynx, alveolar ridge, maxilla) were excluded. A total of 61 patients met these criteria.

### 8.3.3. Bioinformatics

RNA read count data was preprocessed by filtering low expression genes to obtain an approximately Gaussian distribution of Log ₂CPM values. Filtered read count data were then normalized using the trimmed means of M values methods provided in the R edgeR package.(36) The Limma-voom pipeline was used for all subsequent differential expression analysis.(37) Classifiers used the nearest centroid method, and were defined and cross validated using the R cancerclass package.(38)

To construct a high-performance RNA-based classifier for NF-κB activity in HPV+ HNSCC, we employed a centroid classifier, trained on high confidence class members. Preliminary groups of NF-κB active and inactive tumors were assigned by mutational status. Specifically, all tumors with deep deletions (Gistic value=−2) or mutations (missense, nonsense, frame shift) in the NF-κB regulator genes TRAF3 and CYLD were considered NF-κB active, and other tumors inactive. An initial differential expression was performed between these preliminary groups, and a classifier defined, based on the top 100 genes ranked by p-value. High confidence class members were defined as having correct initial assignment and having RNA expression values very similar to the class-defining average of expression (less than 0.25% of the inter-centroid distance). The gene set and classifications were then improved with a machine learning (filtering) procedure, in which tumors initially misclassified or were more than 0.25% away from a centroid were temporarily removed (filtered). Then the filtered data were then used for differential expression and construction of a final classifier. The top 50 genes (by p-value) were selected for this final classifier based on lack of improvement in the receiver operator characteristic with the addition of more genes. Adjusted p-values (multiple comparison correction per the LIMMA package) were calculated and reported. This final classifier had perfect performance on leave-one-out-cross validation. All tumors in the HPV+ HNSCC cohort were then classified according to this final classifier (nearest centroid method) for correlation with clinical and genomic data. Sample classifications were further tuned by setting an empiric threshold for NF-κB activity at the distance of the frameshift or nonsense TRAF3/CYLD mutation farthest from the NF-κB active centroid.

To identify potentially biologically relevant autocorrelated gene sets or gene expression modules (39), the WGCNA algorithm was applied to the above-described RNA expression data, filtered to the top ˜13,000 genes to limit computational intensity. (WGCNA: an R package for weighted correlation network analysis (40). Default parameters according to recommendations from the WGCNA package authors were used unless otherwise noted. The soft threshold network was constructed calculating a scale-free topology fit index for powers ranging from 4-20. The final scale-free network was constructed with soft power set to 6.

Raw RNAseq reads were analyzed for evidence of viral integration using the ViFi package (41). Viral genes expression was also quantified using Salmon (42) and the HPV16 A1 genotype, RefSeq NC_001526.4.

### 8.3.4. Survival Analysis

Clinical data, specifically progression-free interval (PFI), were extracted from Liu et. al. across the full cohort (n=61).(32) We note that the values for PFI from Liu et al were very similar or identical (but included four more cases) when compared to recurrence-free survival (RFS) data available from Broad Firehose Portal. (30) Survival statistics were generated with the R survival package (v3.2-7) and visualized with the R survminer package (0.4.8). p-values represent log-rank test.

### 8.3.5. Gene Set Enrichment Analysis

Ranked gene lists were created using the signal to noise ratio for the change in expression between two groups of interest as defined in the popular GSEA software package distributed by the Broad Institute.(43, 44) Hallmark signatures from the MiSigDB were used as gene sets of interest.(45) GSEA testing and related multiple comparison testing were performed with the R fgsea package.(46) Hypergeometric (gene ontology) enrichment analysis was performed for the derived WGCNA modules using the EnrichR package with default parameters (47). All results were corrected for multiple comparisons by the EnrichR pipeline, and adjusted p-values were considered significant if adjusted p<0.05.

### 8.3.6. Evaluating the TOGA Mutational Landscape

The TRAF3/CYLD mutational loci and type were assessed across HPV+ HNSCC tumors. TRAF3 genetic alterations were predominantly deep deletions as well as two truncations; these alterations preclude translation of the TRAF3 ubiquitin ligase enzymatic domain resulting in this NF-κB overactive phenotype. Similarly, CYLD alterations included deep deletions and truncations occurring prior to its de-ubiquitinase functional domain.(1) In both cases, protein loss of function is evident, leading to unchecked NF-κB activation. However, two novel CYLD missense mutations (N300S and D618A) with unknown functional significance were discovered, demanding further functional appraisal.

### 8.3.7. Modeling the Novel CYLD Missense Mutations

Employing the QuikChange II-E Site-Directed Mutagenesis Kit (Agilent #200523) per the manufacture's protocol, a wild-type Flag-HA-CYLD expression vector (48) (Addgene #22544) was mutated to reflect the two novel CYLD missense mutations, N300S and D618A. Synthetic forward and reverse oligonucleotide primers (Sigma-Aldrich) were designed to harbor the desired point mutation with high CYLD binding affinity in the region of interest. To create the N300S CYLD mutation, forward primer ACATCAGTGATATCATCCCAGCTTTAT (SEQ ID NO. 1) and reverse primer GCAATAGAATTGTACTTTCAACACACG (SEQ ID NO. 2) were used. To develop the D618A CYLD mutation, gggtctaagtaacacagtggccagaacagaactaaaagc (SEQ ID NO. 3) and gcttttagttctgttctggccactgtgttacttagaccc (SEQ ID NO. 4) were used for the forward and reverse primers, respectively. Sanger sequencing performed by Eton Bioscience (San Diego, CA) confirmed targeted mutation success.

### 8.3.8. Creation of CYLD Knockout Mammalian Cells

Co-transfection of CYLD CRISPR/Cas KO (Santa Cruz #sc-400882-KO-2) and CYLD HDR (Santa Cruz #sc-400882-HDR-2) plasmids were used per manufacture's protocol to develop CYLD knockout U2OS cells. U2OS was chosen as the parental cell based on known wild-type TP53 and Rb expression, characteristic of HPV+ HNSCC disease. (49) Cells were grown in 5% CO2 at 37° C. in DMEM (Genesee #25-501N) supplemented with 10% FBS (Genesee #25-514H) and 1% each of penicillin-streptomycin (Genesee #25-512), non-essential amino acids (Genesee #25-536), and glutamine (Genesee #25-509). KO CYLD cell media was further supplemented with lug/ml puromycin (InvivoGen ant-pr-1) used to select for CRISPR-Cas9 clones. Confirmation of CYLD knockdown was performed with Western blot and a luciferase NF-κB functional assay.

### 8.3.9. Western Blot

Cells were collected by trypsinization and lysed in radioimmunoprecipitation assay (RIPA lysis buffer (Sigma) with the addition of protease inhibitors (Roche) and phosphatase inhibitors (Sigma) for 15 minutes on ice. Lysates were then mechanically homogenized with an 18-gauge syringe and insoluble material was removed by centrifugation at 14,000 rpm for 15 minutes at 4° C. Protein concentration was determined using Qubit assay (Invitrogen). Twenty micrograms of total protein were mixed with 2X loading Laemmli buffer (Biorad) supplemented with DTT (Sigma) and incubated for 10 minutes at 95° C. Proteins were separated in 4% to 20% Tris-glycine polyacrylamide gels (Mini-PROTEAN; Bio-Rad) and electrophoretically transferred onto polyvinylidene fluoride membranes. Membranes were blocked with 3% BSA in PBS and incubated with primary antibodies against CYLD (Santa Cruz) and phospho-p65 (Cell Signaling) as well as control primary antibodies against GAPDH (Santa Cruz). Secondary antibodies were conjugated with horseradish peroxidase (Cell Signaling). After sequential washes in TBST buffer, a chemiluminescent HRP substrate was applied to the membrane and signals were immediately visualized using a ChemiDoc Bio-Rad imager.

### 8.3.10. In Vitro NF-κB Functional Evaluation

U2OS and U20S CYLD KO cells were plated in a 96 well plate at 5×10⁴ cells/100 μl/well. After 24 hours, cells were co-transfected with a 3κB-conA-luciferase expression vector (a generous gift from Dr. Neil Perkins of the University of Dundee, Dundee, UK) and either a CYLD wild-type, CYLD N300S, CYLD D618A, or an empty expression vector using a lipofectamine 2000 (Thermo Fisher #11668030) system per manufacturer's protocol. Forty-eight hours following transfection, cells were lysed and luciferin was applied per manufacturer's protocol (Promega #E1501). Luciferase activity was measured using Promega GloMax Explorer.

### 8.3.11. Data Availability Statement

Raw TCGA data were obtained from NCBI dbGaP (the Database of Genotypes and Phenotypes) Authorized Access system with dbGaP permission.

### 8.4. Results

### 8.4.1. Development of the NF-κB Activity Classifier (NAC)

We previously reported that TRAF3 and CYLD alterations correlated with NF-κB activation and with survival in HPV+ HNSCC (13). Given the prominent role that NF-κB plays in tumorigenesis, we hypothesized that classifying these tumors based on NF-κB activity may improve correlation with outcome since tumors lacking defects in TRAF3 and CYLD may have unrecognized mechanisms driving constitutive NF-κB activity. The role of NF-κB as a transcription factor prompted us to use RNA expression data to more directly measure NF-κB activity. Taking advantage of our finding that TRAF3 and CYLD mutations correlated with outcome and NF-κB activity in the TCGA HNSCC HPV+ cohort (1), TCGA expression data were first grouped by the presence of a known TRAF3 or CYLD defect and the top 100 differentially expressed genes identified. As anticipated, gene set enrichment analyses demonstrated a high enrichment score (>0.3) for NF-κB target genes (FIG. 4, grey line) and several notable NF-κB target genes were differentially expressed—TRAF2, NF-κB2, BIRC3, and MAP3K14.

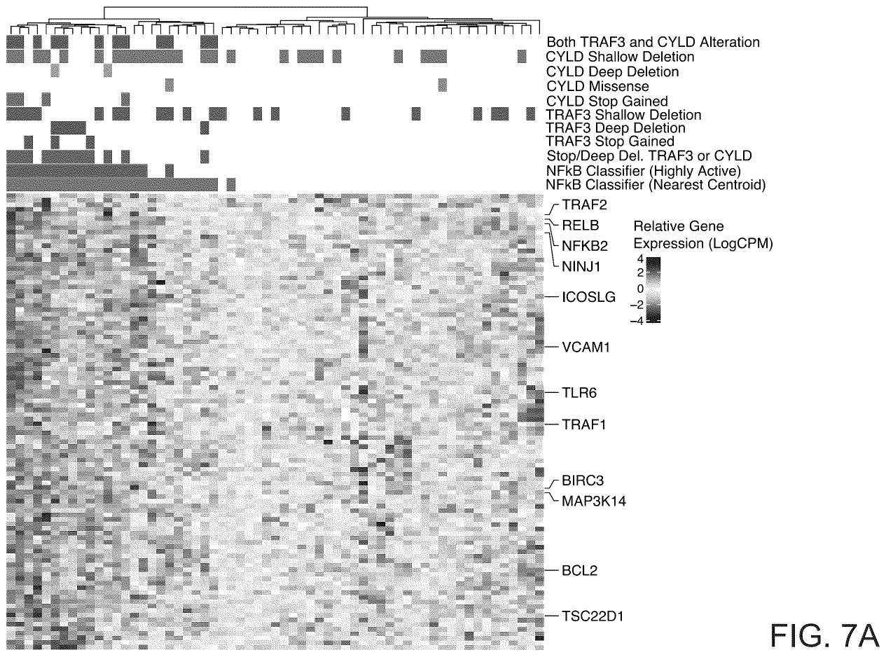

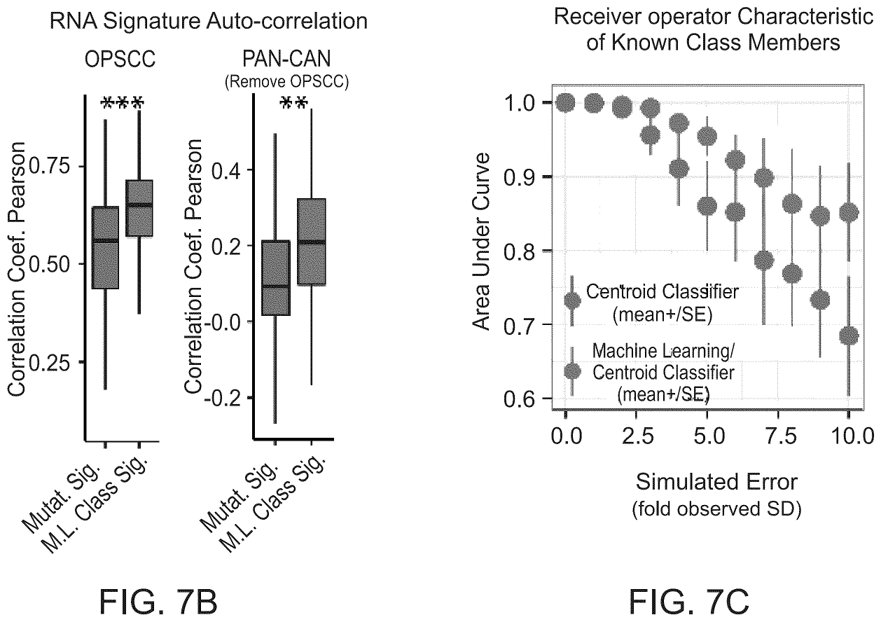

Machine learning techniques (see Methods) were used to refine the signature resulting in a set of 50 key genes dubbed the NF-κB Activity Classifier Gene Signature (***Supplemental Table 1). Using the NF-κB Activity Classifier (nearest centroid), all tumors were then given a final classification to identify tumors with high NF-κB activity (FIG. 1, track 1). Interestingly, many samples without a loss of function alteration (deep deletion, nonsense/frameshift mutation) in either TRAF3 or CYLD (FIG. 7A, track 3) were included in the NF-κB active group (see also ***Supplemental Table 2). In order to identify a set of tumors with equivalently high activation of NF-κB, as observed with destructive nonsense or frameshift mutations in TRAF3 or CYLD, we also defined a more stringent threshold of NF-κB activation, based on the lowest classifier score observed for the highest confidence destructive alterations (nonsense or frameshift) of TRAF3 or CYLD (see FIG. 1, track 2). Notably, 6 tumors included in this “highly active” NF-κB group also were found to be without deep deletion, frameshift/nonsense mutation of TRAF3 or CYLD, bolstering the utility of an RNA based approach to identify NF-κB activated HPV+ HNSCC tumors.

All tumors harboring simultaneous alterations (including shallow deletions) in both TRAF3 and CYLD were found to be in the NF-κB active group (FIG. 1A, track 11), and two of these tumors were included in the “highly active” NF-κB group. These data suggest that combinations of more subtle changes effecting both TRAF3 and CYLD can contribute to NF-κB activity.

8.4.2. RNA-Based Classification Strengthens the Association with NF-κB Target Gene Expression.

To determine if the NF-κB Activity Classifier enhanced correlation with NF-κB target genes relative to groupings based on TRAF3/CYLD alterations, we performed gene set enrichment analysis using TRAF3/CYLD (missense, nonsense, frame shift) and the highly active NF-κB classification as determined by the NAC. This analysis demonstrated significant enrichment for the Hallmark NF-κB target gene set for both TRAF3/CYLD and highly active NF-κB classifiers (p-value<0.01); however, stratification using the NF-κB Activity Classifier demonstrated stronger enrichment (FIG. 4).

### 8.4.3. Machine Learning (ML) Improves NF-κB Gene Set Properties and Classifier Robustness.

Auto-correlation, or compactness, is a desirable feature of RNA expression signatures since loss of compactness when applied to new datasets can limit their diagnostic utility(39). To begin determining compactness of the NF-κB activity gene set (signature) auto-correlation was examined. Pearson correlation coefficients were improved after the machine learning procedure, both in the HNSCC tumors used for deriving the gene set; as well as across all tumor types included in the TCGA pan-cancer atlas (FIG. 7B). Since clinical expression datasets might be expected to have more error compared to that collected for TCGA, we also considered how robust our classifications were to increasing noise of measurement. To examine this, we calculated the area under the receiver-operator characteristic curve (AUC) for the original and ML improved classifier with increasing levels of (random) simulated error applied to the RNA expression data. The ML-improved classifier had higher AUC values at higher levels of noise. It maintained a median AUC of >0.95 even with a five-fold increase in error as compared to the original RNA data from TCGA (FIG. 7C). Taken together these analyses illustrate the favorable properties of our NF-κB activity gene set (signature), as well as a high-degree of robustness of the nearest centroid classifications based on these genes.

### 8.4.4. Weighted Gene Correlation Network Analysis Identifies an NF-κB Associated Gene Expression Module in HPV+ HNSCC.

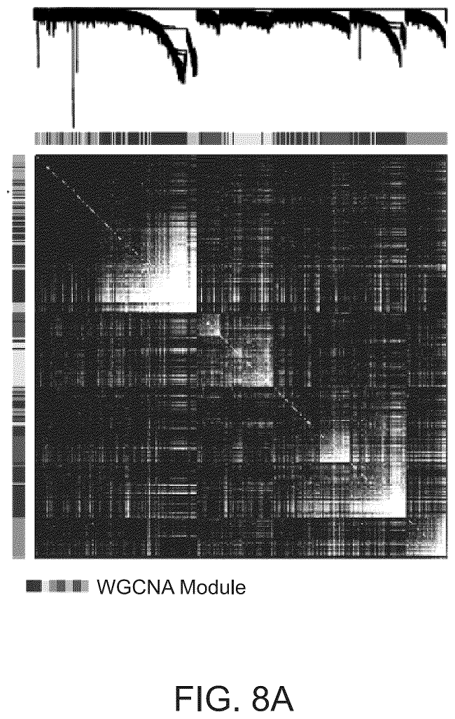

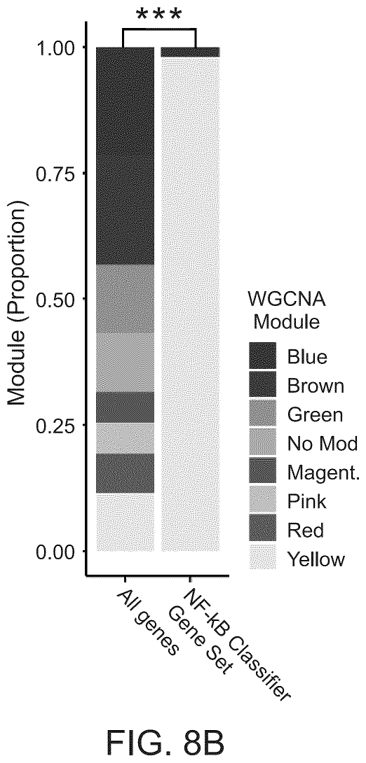

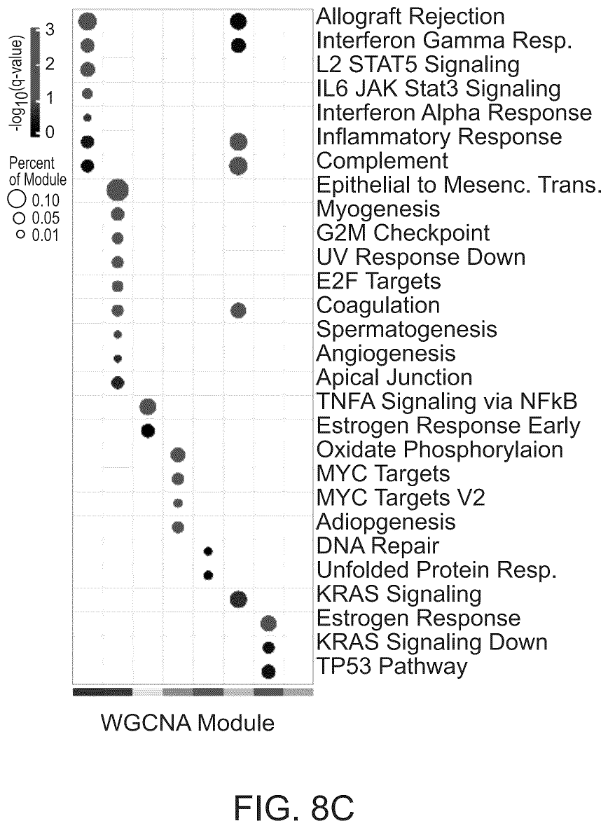

To determine the relationship of our final classifier genes signature (50 genes) to other aspect of the cellular gene expression and signaling, we performed weighted gene correlation network analysis (WGCNA). In order to render required processor times tractable, only the 13,000 most highly expressed genes were included in the WGCNA analysis, excluding 2 of the 50 classifier genes. This unguided discovery approach identified 7 sets (or modules) of highly autocorrelated genes; the relative size and correlative dissimilarity between the modules are displayed in FIG. 8A. These modules were then screened for (hypergeometric) enrichment of the established hallmark gene sets from the MiSig database (FIG. 8C). Interestingly, one module (“yellow”) was found to be most associated with NF-κB target gene expression by both p-value and fraction of module genes in the test signature (FIG. 8C). Of note, no other modules were enhanced for NF-κB targets. Furthermore, 47 of 48 signature genes included in the WGCNA analysis were found to be in the “yellow” module (FIG. 8B, Table 3 for comprehensive gene set list of WGCNA modules, and Table 4 for related hypergeometric enrichment analysis). The “yellow” module was also associated with early estrogen receptor (ER) signaling, and the “magenta” module was associated with estrogen response genes (FIG. 8C).

8.4.5. Expression-based Classification Improves Correlation with Survival

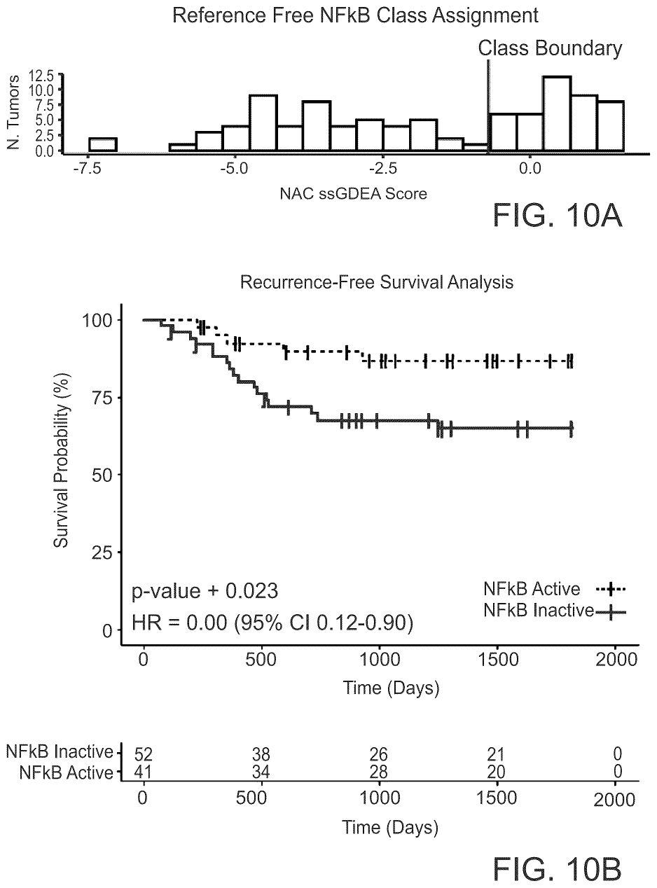

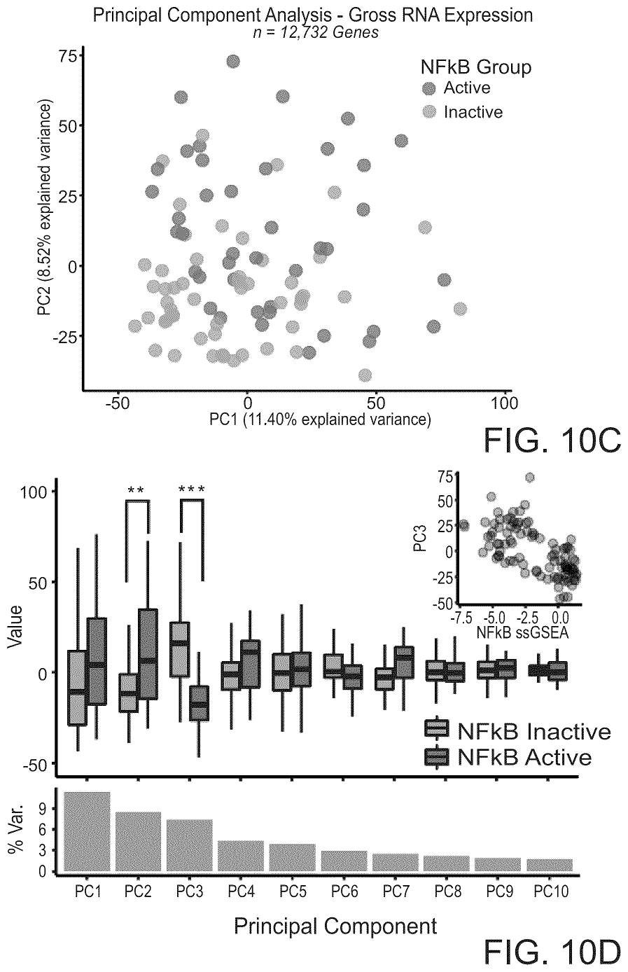

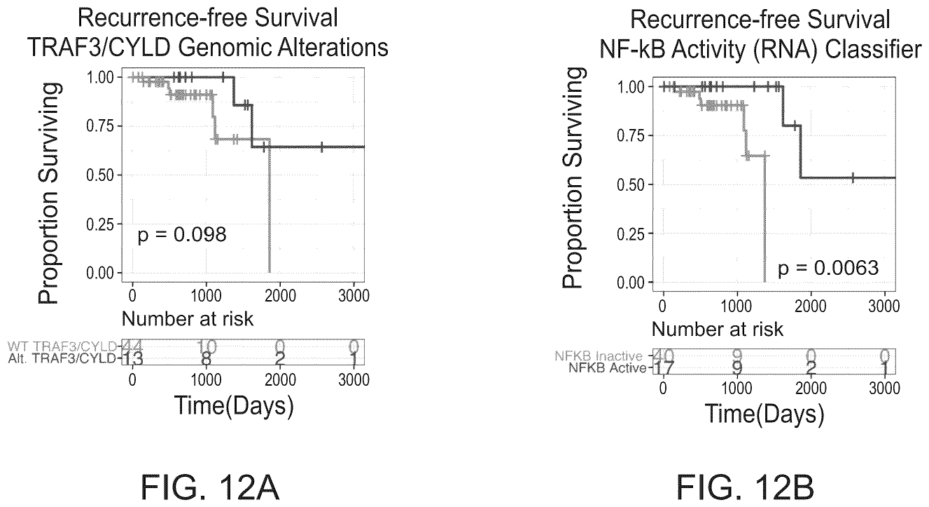

Clinical outcomes for the TCGA HPV+ HNSCC cohort were assessed with PFI, available for all TCGA samples from Liu et al.(32) Kaplan-Meier survival curves were created for samples stratified by the presence of a TRAF3 or CYLD genomic alteration (FIG. 10A) and using the NF-κB Activity Classifier (FIG. 10B). In both cases, a survival advantage was apparent for this distinct disease phenotype. However, the NF-κB Activity Classifier was associated with a larger hazard ratio (HR=6.8) and statistically significant difference in PFI (p=0.01) (FIG. 5C-5D). Although fewer tumors (n=57) were annotated for recurrence-free survival (RFS), classification of NF-κB active tumors using the NAC also correlated with improved RFS (FIG. 12, p-value=0.006).

8.4.6. NF-κB Activity Correlates with HPV Viral Integration Status

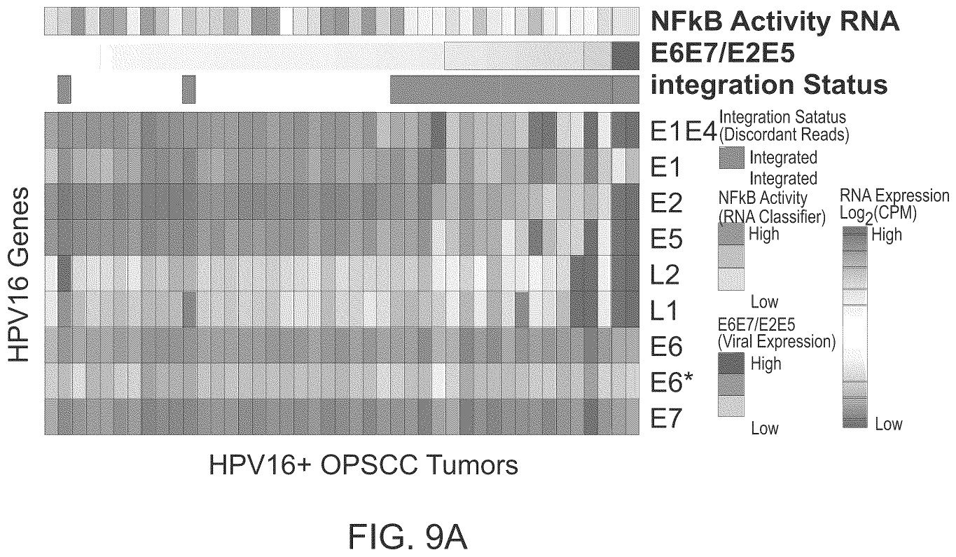

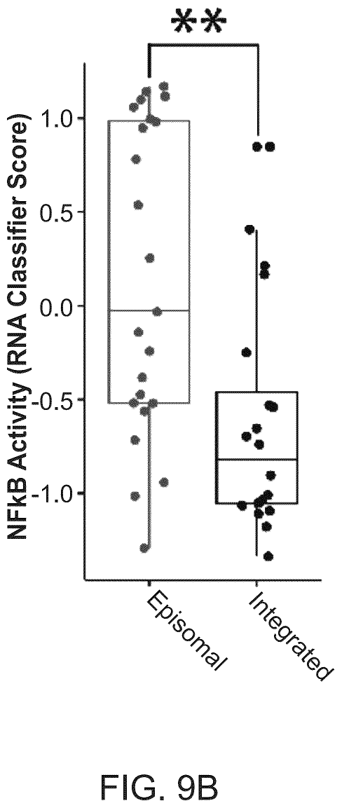

We previously reported that somatic alterations in TRAF3 and CYLD were associated with lack of viral integration in HPV+ HNSCC. To examine if our RNA-based estimates of NF-κB activity also correlated with viral integration, we first determined integration based on discordant read pair mapping-sequences that mapped to both the human and HPV viral genomes. Tumors were only considered integrated if multiple discordant read pairs mapped to similar areas of the human and viral genomes (41). The ratio of expression of viral genes E6 and E7 to E1 and E2 has been used as a surrogate marker for integration (50), however, in our hands the ratio of E6/E7 to E2/E5 was more correlated to integration identified by discordant read pairs (see FIG. 9A). Comparison of RNA-based NF-κB activity (classifier scores) demonstrated a strong relationship to viral integration status, with episomal tumors having much higher median NF-κB activity (FIG. 9B, p-value<0.001).

8.4.7. NF-κB Activity Correlates with Patient Outcome in an Independent Validation Dataset

To validate the prognostic value of the NF-κB activity classifier, we queried the literature for suitable datasets, finding one study with suitable RNA expression (RNAseq) data and clinical annotation (51)(See Table 5). Since somatic mutational data was not available in this RNA expression dataset, we applied single-sample gene set enrichment analysis (ssGSEA) to score each tumor for NF-κB activity using the NAC gene signature (FIG. 10A). Interestingly, NAC gene signature ssGSEA scores were distributed in a bimodal pattern, enabling empiric classification of tumors based on a simple threshold roughly dividing the two distributions (FIG. 10A). Recurrence-free survival analysis based on these groups demonstrated improved survival for the NF-κB active group (FIG. 10B). We also queried an additional related dataset from a different institution which included patients primarily treated with surgery, but no significant difference in recurrence free survival was noted in this dataset (52, 53).

### 8.4.8. NF-κB Activity Classifier RNA Signature Maintains Favorable Properties in an Independent Validation Dataset.

To investigate the relationship to of the NF-κB activity gene signature to global variability in (human) gene expression, we performed principal component analysis (FIG. 10C-10D). NF-κB activity groups were not strongly correlated with the principal component associated with the greatest degree of variability in the dataset (PC1). Among the 10 top principal components, only PC3 (and to a lesser degree PC2), were associated with the NF-κB activity groups (FIG. 10C-10D). Taken together, these results suggest that variability in the expression of the NF-κB activity gene signature is specific, and not simply a reflection of gross data variability. Principal component (PC3) and NAC gene signature ssGSEA scores were strongly correlated (FIG. 4D inset, Pearson's Rho=−0.63, p-value=5*10\hat{ }−12), which suggests that expression of NF-κB activity signature genes can be reliably identified independent of scoring metric, which is a key feature of high-quality gene signatures (39).

8.4.9. CYLD Missense Mutants are not Associated with Loss of Function

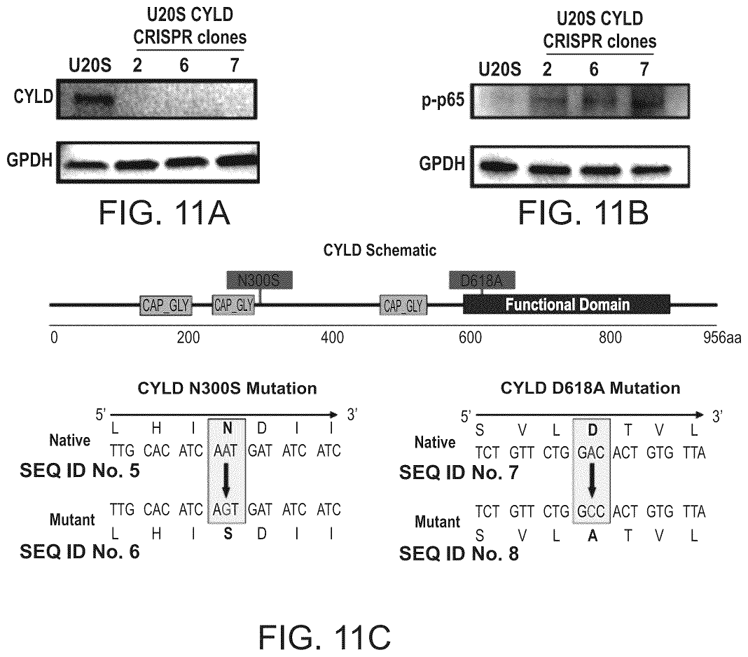

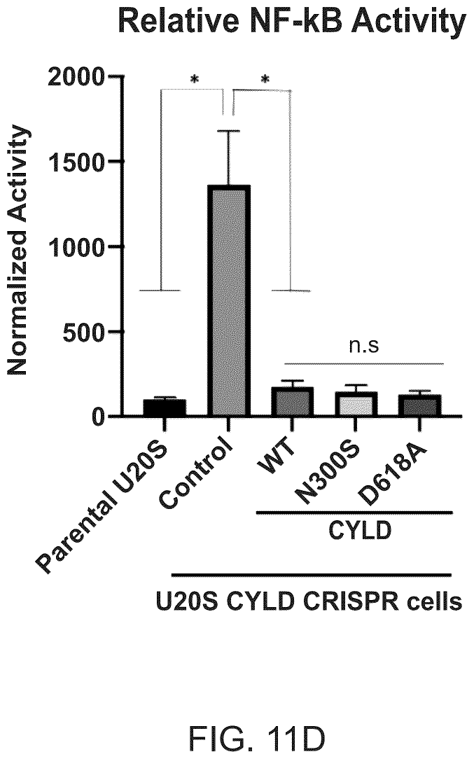

Stratification of tumors by the NF-κB Activity Classifier found that only one of the two identified CYLD missense mutations was associated with increased NF-κB activity (FIG. 7A, track 8). Considering the missense mutation in the “highly active” NF-κB group had concurrent shallow deletions in both TRAF3 and CYLD, we wanted to evaluate the functional consequences of the CYLD missense mutations. To test CYLD activity, we developed CYLD knockout in U2OS osteosarcoma cells and confirmed loss of CYLD expression and activation of NF-κB by phosphor-p65 immunoblotting (FIG. 11A-11B). To test activity of CYLD missense mutations identified from HPV+ HNSCC in TCGA, site-directed mutagenesis was used to create expression plasmids and activity compared to wild-type CYLD in CYLD knockout U2OS cells (FIG. 11C). As expected, CYLD knockout cells showed significantly elevated NF-κB activity compared to parental cells (FIG. 11D). Interestingly, both N300S or D618A mutant CYLD proteins were as efficient in inhibiting NF-κB transcriptional activity as wild-type CYLD (FIG. 11D). These data suggest that N300S and D618A CYLD missense mutations are not inactivating mutations and are not responsible for NF-κB activation.

### 8.5. Discussion

HNSCC is a devastating disease with an increasing global incidence due to human papillomavirus and continued consumption of carcinogens.2, 7, 10) In contrast to HPV-negative HNSCC, HPV-mediated tumors are more susceptible to contemporary treatment paradigms which also leads to improved patient survival.(54) However, HPV+ HNSCC survivors are frequently burdened with significant side effects including pain; neck muscle stiffness; dry mouth; and difficulty with speech, eating/drinking, and breathing. Efforts to reduce these significant quality-of-life effects have triggered multiple trials of treatment de-escalation. In these trials, patients are selected for deintensified treatment based on patient factors like smoking status, histological characteristics following an ablative procedure, or response to induction chemotherapy.(55) Given that methods to identify patients for deintensified therapy are imperfect, our improved classifiers may serve as prognostic biomarker to help clinicians with therapeutic decisions.

Recent work examined genomic characteristics of the tumor that could be used prior to treatment to prognostically stratify patients. Somatic mutations or deletions in TRAF3 or CYLD identified a subset of HPV+ HNSCC associated with improved outcome.(1, 13, 14) Increasing evidence demonstrates these somatic mutant tumors identify a distinct clinical entity given notable molecular, histopathologic, and outcome differences.(3, 13, 56) Regarding function, TRAF3 is a ubiquitin ligase that regulates numerous receptor pathways, ultimately functioning to negatively regulate both canonical and non-canonical NF-κB pathways.(57) Similarly, CYLD inhibits the NF-κB pathway in its role as a deubiquitinase.(58) Inactivation of TRAF3 or CYLD results in activation of NF-κB producing robust downstream effects as demonstrated by significant RNA expression changes amongst mutant TRAF3/CYLD tumors (FIG. 7A).(59)

Initially, NF-κB was thought to protect cells through anti-viral activities through induction of immune response genes.(60) However, it is now apparent many viruses rely on or even induce aberrant NF-κB activity to promote host cell survival and proliferation, thereby supporting the viral lifecycle and thus viral gene expression.(59-61) Previous groundbreaking work revealed that NF-κB overactivation favors carcinogenesis with EBV and HIV-mediated disease with a fundamental role of constitutive NF-κB signaling in EBV tumorigenesis.(19, 21-24) When aberrantly activated, NF-κB is thought to stabilize the EBV episome while suppressing the lytic cycle.(19, 21, 62) Interestingly, the HPV+ HNSCC TCGA cohort demonstrated a trend between tumors with TRAF3/CYLD mutations and maintenance of episomal HPV, whereas those with wild-type TRAF3/CYLD tended to demonstrate HPV integration.(6, 13) We expand this finding herein by demonstrating that viral integration status is highly correlated to NF-κB activation.

In HPV+ HNSCC, TRAF3 or CYLD mutations correlate with a lack of HPV integration providing insight into their potential role in HPV carcinogenesis in the upper aerodigestive tract.(13) Current knowledge of HPV-induced carcinogenesis is largely derived from study of uterine cervical cancer with the classical model showing persistent infection followed by HPV genome integration leading to increased expression of HPV oncoproteins.(63) The absence of HPV integration in a substantial portion of HNSCC coupled with constitutive NF-κB activation as we show here (FIG. 9A-9B), suggests that HPV carcinogenesis in the upper aerodigestive tract may be driven by maintenance of episomal HPV. Interestingly, HPV genome integration has consistently associated with worse survival in these tumors (50, 64, 65).

As clinicians search for markers to predict outcome in HPV+ HNSCC, smoking history and tumor classification are the only criteria that are currently used prior to therapy (66). As these markers are imperfect, several groups are exploring characteristic of HPV+ HNSCC that correlate with outcome. Tools incorporating multiple clinical, demographic, and performance status data have been developed as a prognosticator of overall and progression free survival (67). Once identified, addition of molecular tumor characteristics in these nomograms may improve their predictive accuracy. In addition to the TRAF3/CYLD mutation and HPV genome integration status, others have used gene expression profiles to identify subtypes or to correlate with survival in HPV− associated HNSCC (68). Both supervised and unsupervised expression patterns that correlated with survival identified genes associated with inflammation in the good prognostic group.

An unexpected recent finding revealed that estrogen receptor (ER) expression correlated with improved survival in HPV+ HNSCC (69). Interestingly, the correlation of ER expression with survival was limited to the group of patients treated non-surgically, corresponding to validation of our findings in patients treated primarily with radiation with or without chemotherapy, but not in the cohort treated primarily with surgery.

The relationship between ER and NF-κB signaling is complex, with initial studies focusing on inflammatory signaling where NF-κB is pro-inflammatory, and ER is anti-inflammatory. These studies found that ER expression and signaling inhibited NF-κB (70) explained mechanistically through estrogen stabilization of IκBα(71). Later studies unveiled the complexity of the interaction in inflammatory signaling with conflicting results showing that ER signaling enhanced NF-κB activity in macrophages and T cells, suggesting that the interaction between ER and NF-κB signaling may depend on cellular context (72, 73). In breast cancer, the interaction between ER and NF-κB has also been reported as both antagonistic and synergistic with examples of NF-κB down-regulating ER expression, but also of increasing ER recruitment to DNA and transcription in the presence or absence of estrogen (74). Given that both ER expression and loss of TRAF3 portend improved prognosis in HPV+ HNSCC, description that ER-alpha stimulation depletes cells of TRAF3 via ubiquitination provides a potential mechanistic connection of these findings (75). As far as we are aware, the cross talk between NF-κB and ER signaling is not described in the presence of HPV and particularly, not in HPV HNSCC. Although our presented work cannot determine causality, the WGCNA analysis (FIG. 8A-8C) suggests a positive correlation between ER signaling NF-κB activity in HPV+ OPSCC, with the “yellow” module being enriched for both NF-κB and early estrogen response genes. Also, the nearest neighbor (relative to “yellow”) “magenta” module was also enriched for estrogen response genes (FIGS. 8A and 8C).

Use of multi-variable predictor models is gaining recent clinical traction since these tools provide a more comprehensive assessment of the intratumoral environment.(25-27) In our case, we hypothesized that undefined alterations in addition to TRAF3 or CYLD gene defects are in play to activate NF-κB in HPV+ HNSCC. Querying only TRAF3 or CYLD defects would be blind to these alternative NF-κB activating strategies leading to imperfect tumor classification. Indeed, the NF-κB Activity Classifier identified several NF-κB active tumors excluded by genomic analysis of TRAF3/CYLD (FIG. 7A). Reassuringly, tumors with deep deletions in either TRAF3 or CYLD, or a truncating mutation proximal to the proteins' functional domain were consistently included in the “active” NF-κB category. Conversely, tumors with isolated shallow deletions tended to be in the NF-κB “inactive” category. However, the NF-κB Activity Classifier identified many samples in the NF-κB “active” category that do not follow this clear-cut pattern, in particular identifying that simultaneous shallow deletion of TRAF3 and CYLD in a tumor correlated with NF-κB activity. The finding that all tumors with shallow co-occurring deletions in both TRAF3 and CYLD were included in the NF-κB “active” group suggests a functional interaction of TRAF3 and CYLD in these tumors. On the other hand, our direct testing revealed that missense mutations of CYLD found in HPV+ HNSCC do not lose ability to regulate NF-κB (FIG. 11A-11D). One tumor with the D618A CYLD mutation was classified as NF-κB highly active, but this tumor also harbored simultaneous shallow TRAF3 and CYLD deletions. Accuracy of the NF-κB Activity Classifier to identify NF-κB activity in HPV+ HNSCC was suggested through its improved correlation with patient outcome compared to segregating tumors based on TRAF3 or CYLD defects. From the biological perspective, this finding also supports the notion that NF-κB activation and related changes in gene expression may be the key factor determining the biological differences previously reported for TRAF3/CYLD mutant HPV+ HNSCC, rather than other potential effects of these variants.(13)

Widespread use of genomic technologies has challenged the larger field of cancer biology to identify which innovations are more relevant to inform patient care.(76) Our previous work identified the potential value of TRAF3 and CYLD gene defects to predict outcomes in HPV+ HNSCC.(13) Herein, we demonstrate that an RNA-based classifier trained on tumors harboring these mutations may improve prognostic classification (FIG. 4A-4D and FIG. 10B). As clinical algorithms for treatment de-escalation are not presently informed by prognostic biomarkers, the possibility of an RNA-based approach for determining NF-κB related prognostic groups is quite relevant. Furthermore, RNA-based gene expression profiling has the potential to synthesize disparate observations related to prognosis in HPV+ OPSCC. Specifically, other groups have found that ER-alpha expression is prognostic (77) and we find that ER signaling is correlated with NF-κB activity (FIG. 8A-8C). Similarly, we find that NF-κB activity assessed by RNA expression is highly related to viral integration status which has also been put forward as a prognostic marker in HPV+ OPSCC (50). Future work will be needed optimize RNA-based biomarkers which represent the full prognostic potential of all relevant pathways including NF-κB signaling, ER signaling and viral oncogene expression, but such a synthetic approach is likely possible based on the correlations between these transcriptional pathways we have identified.

Although success of translating gene expression sets from translational and experimental studies has only limited success to date, our analyses support the biological and clinical utility of the gene set we have developed (78). The NF-κB related gene signature and classifier developed in this work demonstrate many desirable properties that suggest that they may be translatable across multiple cohorts and RNA quantification technologies(39). Using the TCGA data set, we confirmed the robustness of RNA-based classifications in the presence of high levels of noise (FIG. 4, FIG. 7A-7C). The NF-κB RNA gene set was highly auto-correlated and distinct from other transcriptional programs in HPV+ OPSCC (FIG. 7B, FIG. 8A-8B). Using a second cohort we directly validated the utility of our gene set outside of the original training data (FIG. 10A-10D). In the validation cohort, a bimodal expression of the NF-κB gene signature as measured by ssGSEA suggests that indeed two biological groups (NF-κB high and low) are a feature of HPV+ OPSCC, and these groups also correlated with RFS in this second Data set. Furthermore, the NF-κB gene signature expression was not correlated to 8/10 top principal components demonstrating that the gene set does not simply report gross (transcriptome wide) changes in gene expression. Conversely, the very strong correlation to PC3 suggests that gene set remains compact when applied to new Data sets, and can likely be quantified by many metrics (FIG. 10C-10D).

This report validates and expands on our findings that significant expression changes related to NF-κB activity occur in the subset of HPV+ HNSCC tumors marked by TRAF3 or CYLD mutations. We are planning future studies investigating the importance of “long-tail” mutations in the NF-κB pathway which might further illuminate the origins of NF-κB dysregulation in HPV+ HNSCC.

Using the NF-κB Activity Classifier, we demonstrate a more sensitive stratification approach than relying on single gene mutations (i.e. TRAF3/CYLD mutation status) perhaps suggesting the algorithm's potential for prospective treatment personalization of HPV+ HNSCC.

A major discovery in the recent past is that HPV associated HNSCC have improved survival compared to tobacco associated tumors. This finding coupled with advancements in tumor genomic analysis definitively established HPV+ and HPV-negative HNSCC as distinct tumors. Similarly, we noted genomic differences amongst subclasses of HPV+ HNSCC and found that defects in TRAF3 and CYLD correlated with survival. Here we present data that these subclasses may also be identified by direct assessment of NF-κB activity; as demonstrated by gene expression differences highlighted by the NF-κB Activity Classifier. Since clinicians are exploring therapeutic deintensification for HPV+ HNSCC, identifying patients with good or poor prognosis using the NF-κB Activity Classifier may be useful to guide therapeutic decisions.

### 9. REFERENCES EXAMPLE 2

1. Cancer Genome Atlas N. Comprehensive genomic characterization of head and neck squamous cell carcinomas. Nature. 2015; 517(7536):576-82. Epub 2015 Jan. 30. doi: 10.1038/nature 14129. PubMed PMID: 25631445; PMCID: PMC4311405.
2. Johnson D E, Burtness B, Leemans C R, Lui V W Y, Bauman J E, Grandis J R. Head and neck squamous cell carcinoma. Nat Rev Dis Primers. 2020; 6(1):92. Epub 2020 Nov. 28. doi: 10.1038/s41572-020-00224-3. PubMed PMID: 33243986.
3. Williams E A, Montesion M, Alexander B M, Ramkissoon S H, Elvin J A, Ross J S, Williams K J, Glomski K, Bledsoe J R, Tse J Y, Mochel M C. CYLD mutation characterizes a subset of HPV-positive head and neck squamous cell carcinomas with distinctive genomics and frequent cylindroma-like histologic features. Mod Pathol. 2021; 34(2):358-70. Epub 2020 Sep. 7. doi: 10.1038/s41379-020-00672-y. PubMed PMID: 32892208; PMCID: PMC7817524.
4 Gillison M L, Akagi K, Xiao W, Jiang B, Pickard R K L, Li J, Swanson B J, Agrawal A D, Zucker M, Stache-Crain B, Emde A K, Geiger H M, Robine N, Coombes K R, Symer D E. Human papillomavirus and the landscape of secondary genetic alterations in oral cancers. Genome Res. 2019; 29(1):1-17. Epub 2018 Dec. 20. doi: 10.1101/gr.241141.118. PubMed PMID: 30563911; PMCID: PMC6314162.
5. Pytynia K B, Dahlstrom K R, Sturgis E M. Epidemiology of HPV-associated oropharyngeal cancer. Oral Oncol. 2014; 50(5):380-6. Epub 2014 Jan. 28. doi: 10.1016/j.oraloncology.2013. 12.019. PubMed PMID: 24461628; PMCID: PMC4444216.
6. Pan C, Issaeva N, Yarbrough W G. HPV-driven oropharyngeal cancer: current knowledge of molecular biology and mechanisms of carcinogenesis. Cancers Head Neck. 2018; 3:12. Epub 2019 May 17. doi: 10.1186/s41199-018-0039-3. PubMed PMID: 31093365; PMCID: PMC6460765.
7. Shiboski C H, Schmidt B L, Jordan R C. Tongue and tonsil carcinoma: increasing trends in the U.S. population ages 20-44 years. Cancer. 2005; 103(9):1843-9. Epub 2005 Mar. 18. doi: 10.1002/cncr.20998. PubMed PMID: 15772957.
8. Viens L J, Henley S J, Watson M, Markowitz L E, Thomas C C, Thompson T D, Razzaghi H, Saraiya M. Human Papillomavirus-Associated Cancers-United States, 2008-2012. MMWR Morb Mortal Wkly Rep. 2016; 65(26):661-6. Epub 2016 Jul. 9. doi: 10.15585/mmwr.mm6526a1. PubMed PMID: 27387669.
9. Herrero R, Castellsague X, Pawlita M, Lissowska J, Kee F, Balaram P, Rajkumar T, Sridhar H, Rose B, Pintos J, Fernandez L, Idris A, Sanchez M J, Nieto A, Talamini R, Tavani A, Bosch F X, Reidel U, Snijders P J, Meijer C J, Viscidi R, Munoz N, Franceschi S, Group IMOCS. Human papillomavirus and oral cancer: the International Agency for Research on Cancer multicenter study. J Natl Cancer Inst. 2003; 95(23):1772-83. Epub 2003 Dec. 5. doi: 10.1093/jnci/djg107. PubMed PMID: 14652239.
10. Chaturvedi A K, Engels E A, Anderson W F, Gillison M L. Incidence trends for human papillomavirus-related and -unrelated oral squamous cell carcinomas in the United States. J Clin Oncol. 2008; 26(4):612-9. Epub 2008 Feb. 1. doi: 10.1200/JCO.2007.14.1713. PubMed PMID: 18235120.
11. Burtness B, Harrington K J, Greil R, Soulieres D, Tahara M, de Castro G, Jr., Psyrri A, Baste N, Neupane P, Bratland A, Fuereder T, Hughes BGM, Mesia R, Ngamphaiboon N, Rordorf T, Wan Ishak W Z, Hong R L, Gonzalez Mendoza R, Roy A, Zhang Y, Gumuscu B, Cheng J D, Jin F, Rischin D, Investigators K-. Pembrolizumab alone or with chemotherapy versus cetuximab with chemotherapy for recurrent or metastatic squamous cell carcinoma of the head and neck (KEYNOTE-048): a randomised, open-label, phase 3 study. Lancet. 2019; 394(10212):1915-28. Epub 2019 Nov. 5. doi: 10.1016/S0140-6736 (19) 32591-7. PubMed PMID: 31679945.
12. Fakhry C, Zhang Q, Nguyen-Tan P F, Rosenthal D, El-Naggar A, Garden A S, Soulieres D, Trotti A, Avizonis V, Ridge J A, Harris J, Le Q T, Gillison M. Human papillomavirus and overall survival after progression of oropharyngeal squamous cell carcinoma. J Clin Oncol. 2014; 32(30):3365-73. Epub 2014 Jun. 25. doi: 10.1200/JCO.2014.55.1937. PubMed PMID: 24958820; PMCID: PMC4195851.
13. Hajek M, Sewell A, Kaech S, Burtness B, Yarbrough W G, Issaeva N. TRAF3/CYLD mutations identify a distinct subset of human papillomavirus-associated head and neck squamous cell carcinoma. Cancer. 2017; 123(10):1778-90. Epub 2017 Mar. 16. doi: 10.1002/cncr.30570. PubMed PMID: 28295222; PMCID: PMC5419871.
14. Cui Z, Kang H, Grandis J R, Johnson D E. CYLD Alterations in the Tumorigenesis and Progression of Human Papillomavirus-Associated Head and Neck Cancers. Mol Cancer Res. 2021; 19(1):14-24. Epub 2020 Sep. 5. doi: 10.1158/1541-7786.MCR-20-0565. PubMed PMID: 32883697.
15. Annunziata C M, Davis R E, Demchenko Y, Bellamy W, Gabrea A, Zhan F, Lenz G, Hanamura I, Wright G, Xiao W, Dave S, Hurt E M, Tan B, Zhao H, Stephens O, Santra M, Williams D R, Dang L, Barlogie B, Shaughnessy J D, Jr., Kuehl W M, Staudt L M. Frequent engagement of the classical and alternative N F-kappaB pathways by diverse genetic abnormalities in multiple myeloma. Cancer Cell. 2007; 12(2):115-30. Epub 2007 Aug. 19. doi: 10.1016/j.ccr.2007.07.004. PubMed PMID: 17692804; PMCID: PMC2730509.
16. Keats J J, Fonseca R, Chesi M, Schop R, Baker A, Chng W J, Van Wier S, Tiedemann R, Shi C X, Sebag M, Braggio E, Henry T, Zhu Y X, Fogle H, Price-Troska T, Ahmann G, Mancini C, Brents L A, Kumar S, Greipp P, Dispenzieri A, Bryant B, Mulligan G, Bruhn L, Barrett M, Valdez R, Trent J, Stewart A K, Carpten J, Bergsagel P L. Promiscuous mutations activate the noncanonical N F-kappaB pathway in multiple myeloma. Cancer Cell. 2007; 12(2):131-44. Epub 2007 Aug. 19. doi: 10.1016/j.ccr.2007.07.003. PubMed PMID: 17692805; PMCID: PMC2083698.
17. Ahmed Z, Afridi S S, Shahid Z, Zamani Z, Rehman S, Aiman W, Khan M, Mir M A, Awan F T, Anwer F, Iftikhar R. Primary Mediastinal B-Cell Lymphoma: A 2021 Update on Genetics, Diagnosis, and Novel Therapeutics. Clin Lymphoma Myeloma Leuk. 2021; 21(11):e865-e75. Epub 2021 Aug. 1. doi: 10.1016/j.clml.2021.06.012. PubMed PMID: 34330673.
18. Mirghani H, Mortuaire G, Armas G L, Hartl D, Auperin A, El Bedoui S, Chevalier D, Lefebvre J L. Sinonasal cancer: Analysis of oncological failures in 156 consecutive cases. Head & neck. 2014; 36(5):667-74. doi: 10.1002/hed.23356. PubMed PMID: 23606521.
19. Chung G T, Lou W P, Chow C, To K F, Choy K W, Leung A W, Tong C Y, Yuen J W, Ko C W, Yip T T, Busson P, Lo K W. Constitutive activation of distinct N F-kappaB signals in EBV-associated nasopharyngeal carcinoma. J Pathol. 2013; 231(3):311-22. Epub 2013 Jul. 23. doi: 10.1002/path.4239. PubMed PMID: 23868181.
20. Li Y, Shi F, Hu J, Xie L, Zhao L, Tang M, Luo X, Ye M, Zheng H, Zhou M, Liu N, Bode A M, Fan J, Zhou J, Gao Q, Qiu S, Wu W, Zhang X, Liao W, Cao Y. Stabilization of p18 by deubiquitylase CYLD is pivotal for cell cycle progression and viral replication. NPJ Precis Oncol. 2021; 5(1):14. Epub 2021 Mar. 4. doi: 10.1038/s41698-021-00153-8. PubMed PMID: 33654169; PMCID: PMC7925679.
21. Santoro M G, Rossi A, Amici C. N F-kappaB and virus infection: who controls whom. The EMBO journal. 2003; 22(11):2552-60. doi: 10.1093/emboj/cdg267. PubMed PMID: 12773372; PMCID: 156764.
22. Li Y Y, Chung G T, Lui V W, To K F, Ma B B, Chow C, Woo J K, Yip K Y, Seo J, Hui E P, Mak M K, Rusan M, Chau N G, Or Y Y, Law M H, Law P P, Liu Z W, Ngan H L, Hau P M, Verhoeft K R, Poon P H, Yoo S K, Shin J Y, Lee S D, Lun S W, Jia L, Chan A W, Chan J Y, Lai P B, Fung C Y, Hung S T, Wang L, Chang A M, Chiosea S I, Hedberg M L, Tsao S W, van Hasselt A C, Chan A T, Grandis J R, Hammerman P S, Lo K W. Exome and genome sequencing of nasopharynx cancer identifies N F-kappaB pathway activating mutations. Nat Commun. 2017; 8:14121. Epub 2017 Jan. 18. doi: 10.1038/ncomms14121. PubMed PMID: 28098136; PMCID: PMC5253631 received research grant and serves the advisory board from Novartis, Hong Kong.
23. Eliopoulos A G, Dawson C W, Mosialos G, Floettmann J E, Rowe M, Armitage R J, Dawson J, Zapata J M, Kerr D J, Wakelam M J, Reed J C, Kieff E, Young L S. CD40-induced growth inhibition in epithelial cells is mimicked by Epstein-Barr Virus-encoded LMP1: involvement of TRAF3 as a common mediator. Oncogene. 1996; 13(10):2243-54. Epub 1996 Nov. 21. PubMed PMID: 8950992.
24. Imbeault M, Ouellet M, Giguere K, Bertin J, Belanger D, Martin G, Tremblay M J. Acquisition of host-derived CD40L by HIV-1 in vivo and its functional consequences in the B-cell compartment. J Virol. 2011; 85(5):2189-200. Epub 2010 Dec. 24. doi: 10.1128/JVI.01993-10. PubMed PMID: 21177803; PMCID: PMC3067784.
25. Miyamoto D T, Lee R J, Kalinich M, LiCausi J A, Zheng Y, Chen T, Milner J D, Emmons E, Ho U, Broderick K, Silva E, Javaid S, Kwan T T, Hong X, Dahl D M, McGovern F J, Efstathiou J A, Smith M R, Sequist L V, Kapur R, Wu C L, Stott S L, Ting D T, Giobbie-Hurder A, Toner M, Maheswaran S, Haber D A. An RNA-Based Digital Circulating Tumor Cell Signature Is Predictive of Drug Response and Early Dissemination in Prostate Cancer. Cancer Discov. 2018; 8(3):288-303. Epub 2018 Jan. 6. doi: 10.1158/2159-8290.CD-16-1406. PubMed PMID: 29301747; PMCID: PMC6342192.
26. Pitroda S P, Pashtan I M, Logan H L, Budke B, Darga T E, Weichselbaum R R, Connell P P. DNA repair pathway gene expression score correlates with repair proficiency and tumor sensitivity to chemotherapy. Sci Transl Med. 2014; 6(229):229ra42. Epub 2014 Mar. 29. doi: 10.1126/scitranslmed.3008291. PubMed PMID: 24670686; PMCID: PMC4889008.
27. McGrail D J, Lin C C, Garnett J, Liu Q, Mo W, Dai H, Lu Y, Yu Q, Ju Z, Yin J, Vellano C P, Hennessy B, Mills G B, Lin S Y. Improved prediction of PARP inhibitor response and identification of synergizing agents through use of a novel gene expression signature generation algorithm. NPJ Syst Biol Appl. 2017; 3:8. Epub 2017 Jun. 27. doi: 10.1038/s41540-017-0011-6. PubMed PMID: 28649435; PMCID: PMC5445594.
28. Shen R, Olshen A B, Ladanyi M. Integrative clustering of multiple genomic data types using a joint latent variable model with application to breast and lung cancer subtype analysis. Bioinformatics. 2009; 25(22):2906-12. Epub 2009 Sep. 18. doi:10.1093/bioinformatics/btp543. PubMed PMID: 19759197; PMCID: PMC2800366.
29. Lerner S P, Weinstein J, Kwiatkowski D, Kim J, Robertson G, Hoadley K A, Akbani R, Creighton C, Group TMIBCAW. The Cancer Genome Atlas Project on Muscle-invasive Bladder Cancer. Eur Urol Focus. 2015; 1(1):94-5. Epub 2015 Aug. 1. doi: 10.1016/j.euf.2014.11.002. PubMed PMID: 28723366.
30. Deng M, Bragelmann J, Kryukov I, Saraiva-Agostinho N, Perner S. FirebrowseR: an R client to the Broad Institute's Firehose Pipeline. Database (Oxford). 2017; 2017. Epub 2017 Jan. 8. doi: 10.1093/database/baw160. PubMed PMID: 28062517; PMCID: PMC5216271.
31. Mermel C H, Schumacher S E, Hill B, Meyerson M L, Beroukhim R, Getz G. GISTIC2.0 facilitates sensitive and confident localization of the targets of focal somatic copy-number alteration in human cancers. Genome Biol. 2011; 12(4):R41. Epub 2011 Apr. 30. doi: 10.1186/gb-2011 Dec. 4-r41. PubMed PMID: 21527027; PMCID: PMC3218867.
32. Liu J, Lichtenberg T, Hoadley K A, Poisson L M, Lazar A J, Cherniack A D, Kovatich A J, Benz C C, Levine D A, Lee A V, Omberg L, Wolf D M, Shriver C D, Thorsson V, Cancer Genome Atlas Research N, Hu H. An Integrated TCGA Pan-Cancer Clinical Data Resource to Drive High-Quality Survival Outcome Analytics. Cell. 2018; 173(2):400-16 e11. Epub 2018 Apr. 7. doi: 10.1016/j.cell.2018.02.052. PubMed PMID: 29625055; PMCID: PMC6066282.
33. Mounir M, Lucchetta M, Silva T C, Olsen C, Bontempi G, Chen X, Noushmehr H, Colaprico A, Papaleo E. New functionalities in the TCGAbiolinks package for the study and integration of cancer data from GDC and GTEx. PLOS Comput Biol. 2019; 15(3):e1006701. Epub 2019 Mar. 6. doi: 10.1371/journal.pcbi. 1006701. PubMed PMID: 30835723; PMCID: PMC6420023.
34. Koboldt D C, Zhang Q, Larson D E, Shen D, Mclellan M D, Lin L, Miller C A, Mardis E R, Ding L, Wilson R K. VarScan 2: somatic mutation and copy number alteration discovery in cancer by exome sequencing. Genome Res. 2012; 22(3):568-76. Epub 2012 Feb. 4. doi: 10.1101/gr. 129684.111. PubMed PMID: 22300766; PMCID: PMC3290792.
35. Goldman M J, Craft B, Hastie M, Repecka K, McDade F, Kamath A, Banerjee A, Luo Y, Rogers D, Brooks A N, Zhu J, Haussler D. Visualizing and interpreting cancer genomics data via the Xena platform. Nat Biotechnol. 2020; 38(6):675-8. Epub 2020 May 24. doi: 10.1038/s41587-020-0546-8. PubMed PMID: 32444850; PMCID: PMC7386072.
36. Robinson M D, McCarthy D J, Smyth G K. edgeR: a Bioconductor package for differential expression analysis of digital gene expression data. Bioinformatics. 2010; 26(1):139-40. Epub 2009 Nov. 17. doi: 10.1093/bioinformatics/btp616. PubMed PMID: 19910308; PMCID: PMC2796818.
37. Law C W, Chen Y, Shi W, Smyth G K. voom: Precision weights unlock linear model analysis tools for RNA-seq read counts. Genome Biol. 2014; 15(2):R29. Epub 2014 Feb. 4. doi: 10.1186/gb-2014-15-2-r29. PubMed PMID: 24485249; PMCID: PMC4053721.
38. Denkert BJDKCvTASSD-EMDC. cancerclass: An R Package for Development and Validation of Diagnostic Tests from High-Dimensional Molecular Data. Journal of Statistical Software, Articles.59(1):1-19. doi: 10.18637/jss.v059.i01.
39. Dhawan A, Barberis A, Cheng W C, Domingo E, West C, Maughan T, Scott J G, Harris A L, Buffa F M. Guidelines for using sigQC for systematic evaluation of gene signatures. Nat Protoc. 2019; 14(5):1377-400. Epub 2019 Apr. 12. doi: 10.1038/s41596-019-0136-8. PubMed PMID: 30971781.
40. Langfelder P, Horvath S. WGCNA: an R package for weighted correlation network analysis. BMC Bioinformatics. 2008; 9:559. Epub 2008 Dec. 31. doi: 10.1186/1471-2105-9-559. PubMed PMID: 19114008; PMCID: PMC2631488.
41. Nguyen N D, Deshpande V, Luebeck J, Mischel P S, Bafna V. ViFi: accurate detection of viral integration and mRNA fusion reveals indiscriminate and unregulated transcription in proximal genomic regions in cervical cancer. Nucleic Acids Res. 2018; 46(7):3309-25. Epub 2018 Mar. 27. doi: 10.1093/nar/gkyl80. PubMed PMID: 29579309; PMCID: PMC6283451.
42. Patro R, Duggal G, Love M I, Irizarry R A, Kingsford C. Salmon provides fast and bias-aware quantification of transcript expression. Nat Methods. 2017; 14(4):417-9. Epub 2017 Mar. 7. doi: 10.1038/nmeth.4197. PubMed PMID: 28263959; PMCID: PMC5600148.
43. Subramanian A, Tamayo P, Mootha V K, Mukherjee S, Ebert B L, Gillette M A, Paulovich A, Pomeroy S L, Golub T R, Lander E S, Mesirov J P. Gene set enrichment analysis: a knowledge-based approach for interpreting genome-wide expression profiles. Proc Natl Acad Sci USA. 2005; 102(43):15545-50. Epub 2005 Oct. 4. doi: 10.1073/pnas.0506580102. PubMed PMID: 16199517; PMCID: PMC1239896.
44. Mootha V K, Lindgren C M, Eriksson K F, Subramanian A, Sihag S, Lehar J, Puigserver P, Carlsson E, Ridderstrale M, Laurila E, Houstis N, Daly M J, Patterson N, Mesirov J P, Golub T R, Tamayo P, Spiegelman B, Lander E S, Hirschhorn J N, Altshuler D, Groop L C. PGC-1alpha-responsive genes involved in oxidative phosphorylation are coordinately downregulated in human diabetes. Nat Genet. 2003; 34(3):267-73. Epub 2003 Jun. 17. doi: 10.1038/ng1180. PubMed PMID: 12808457.
45. Liberzon A, Birger C, Thorvaldsdottir H, Ghandi M, Mesirov J P, Tamayo P. The Molecular Signatures Database (MSigDB) hallmark gene set collection. Cell Syst. 2015; 1(6):417-25. Epub 2016 Jan. 16. doi: 10.1016/j.cels.2015.12.004. PubMed PMID: 26771021; PMCID: PMC4707969.
46. Fast gene set enrichment analysis|bioRxiv [Oct. 29, 2020]. Available from: https://www.biorxiv.org/content/10.1101/060012v2.
47. Xie Z, Bailey A, Kuleshov M V, Clarke D J B, Evangelista J E, Jenkins S L, Lachmann A, Wojciechowicz M L, Kropiwnicki E, Jagodnik K M, Jeon M, Ma'ayan A. Gene Set Knowledge Discovery with Enrichr. Curr Protoc. 2021; 1(3):e90. Epub 2021 Mar. 30. doi:10.1002/cpz1.90. PubMed PMID: 33780170; PMCID: PMC8152575.
48. Sowa M E, Bennett E J, Gygi S P, Harper J W. Defining the human deubiquitinating enzyme interaction landscape. Cell. 2009; 138(2):389-403. Epub 2009 Jul. 21. doi: 10.1016/j.cell.2009.04.042. PubMed PMID: 19615732; PMCID: PMC2716422.
49. Toots M, Ustav M, Jr., Mannik A, Mumm K, Tamm K, Tamm T, Ustav E, Ustav M. Identification of several high-risk HPV inhibitors and drug targets with a novel high-throughput screening assay. PLOS Pathog. 2017; 13(2):e1006168. Epub 2017 Feb. 10. doi: 10.1371/journal.ppat. 1006168. PubMed PMID: 28182794; PMCID: PMC5300127.
50. Koneva L A, Zhang Y, Virani S, Hall P B, McHugh J B, Chepeha D B, Wolf G T, Carey T E, Rozek L S, Sartor M A. HPV Integration in HNSCC Correlates with Survival Outcomes, Immune Response Signatures, and Candidate Drivers. Mol Cancer Res. 2018; 16(1):90-102. Epub 2017 Sep. 21. doi: 10.1158/1541-7786.MCR-17-0153. PubMed PMID: 28928286; PMCID: PMC5752568.
51. Liu X, Liu P, Chernock R D, Kuhs KAL, Lewis J S, Jr., Luo J, Gay H A, Thorstad W L, Wang X. A prognostic gene expression signature for oropharyngeal squamous cell carcinoma. EBioMedicine. 2020; 61:102805. Epub 2020 Oct. 11. doi: 10.1016/j.ebiom.2020.102805. PubMed PMID: 33038770; PMCID: PMC7648117.
52. Haughey B H, Hinni M L, Salassa J R, Hayden R E, Grant D G, Rich J T, Milov S, Lewis J S, Jr., Krishna M. Transoral laser microsurgery as primary treatment for advanced-stage oropharyngeal cancer: a United States multicenter study. Head & neck. 2011; 33(12):1683-94. Epub 2011 Feb. 2. doi: 10.1002/hed.21669. PubMed PMID: 21284056.
53. Jackson R S, Sinha P, Zenga J, Kallogjeri D, Suko J, Martin E, Moore E J, Haughey B H. Transoral Resection of Human Papillomavirus (HPV)-Positive Squamous Cell Carcinoma of the Oropharynx: Outcomes with and Without Adjuvant Therapy. Ann Surg Oncol. 2017; 24(12):3494-501. Epub 2017 Aug. 16. doi: 10.1245/s10434-017-6041-x. PubMed PMID: 28808988.
54. Mehta V, Yu G P, Schantz S P. Population-based analysis of oral and oropharyngeal carcinoma: changing trends of histopathologic differentiation, survival and patient demographics. Laryngoscope. 2010; 120(11):2203-12. Epub 2010 Oct. 13. doi: 10.1002/lary.21129. PubMed PMID: 20938956.
55. Petar S, Marko S, Ivica L. De-escalation in HPV-associated oropharyngeal cancer: lessons learned from the past? A critical viewpoint and proposal for future research. Eur Arch Otorhinolaryngol. 2021. Epub 2021 Feb. 19. doi: 10.1007/s00405-021-06686-9. PubMed PMID: 33599841.
56. Shinriki S, Jono H, Maeshiro M, Nakamura T, Guo J, Li J D, Ueda M, Yoshida R Shinohara M, Nakayama H, Matsui H, Ando Y. Loss of CYLD promotes cell invasion via ALK5 stabilization in oral squamous cell carcinoma. J Pathol. 2018; 244(3):367-79. Epub 2017 Dec. 14. doi: 10.1002/path.5019. PubMed PMID: 29235674.
57. Guven-Maiorov E, Keskin O, Gursoy A, VanWaes C, Chen Z, Tsai C J, Nussinov R. TRAF3 signaling: Competitive binding and evolvability of adaptive viral molecular mimicry. Biochim Biophys Acta. 2016; 1860(11 Pt B):2646-55. Epub 2016 May 22. doi: 10.1016/j.bbagen.2016.05.021. PubMed PMID: 27208423; PMCID: PMC7117012.
58. Mathis B J, Lai Y, Qu C, Janicki J S, Cui T. CYLD-mediated signaling and diseases. Curr Drug Targets. 2015; 16(4):284-94. Epub 2014 Oct. 25. doi: 10.2174/1389450115666141024152421. PubMed PMID: 25342597; PMCID: PMC4418510.
59. Chen T, Zhang J, Chen Z, Van Waes C. Genetic alterations in TRAF3 and CYLD that regulate nuclear factor kappaB and interferon signaling define head and neck cancer subsets harboring human papillomavirus. Cancer. 2017; 123(10):1695-8. Epub 2017 Mar. 16. doi: 10.1002/cncr.30659. PubMed PMID: 28295216; PMCID: PMC5419858.
60. Zhao J, He S, Minassian A, Li J, Feng P. Recent advances on viral manipulation of N F-kappaB signaling pathway. Curr Opin Virol. 2015; 15:103-11. Epub 2015 Sep. 20. doi: 10.1016/j.coviro.2015.08.013. PubMed PMID: 26385424; PMCID: PMC4688235.
61. You R, Liu Y P, Lin D C, Li Q, Yu T, Zou X, Lin M, Zhang X L, He G P, Yang Q, Zhang Y N, Xie Y L, Jiang R, Wu C Y, Zhang C, Cui C, Wang J Q, Wang Y, Zhuang A H, Guo G F, Hua Y J, Sun R, Yun J P, Zuo Z X, Liu Z X, Zhu X F, Kang T B, Qian C N, Mai H Q, Sun Y, Zeng M S, Feng L, Zeng Y X, Chen M Y. Clonal Mutations Activate the N F-kappaB Pathway to Promote Recurrence of Nasopharyngeal Carcinoma. Cancer Res. 2019; 79(23):5930-43. Epub 2019 Sep. 6. doi: 10.1158/0008-5472.CAN-18-3845. PubMed PMID: 31484669.
62. Young L S, Dawson C W. Epstein-Barr virus and nasopharyngeal carcinoma. Chin J Cancer. 2014; 33(12):581-90. Epub 2014 Nov. 25. doi: 10.5732/cjc.014.10197. PubMed PMID: 25418193; PMCID: PMC4308653. 63. Hebner C M, Laimins L A. Human papillomaviruses: basic mechanisms of pathogenesis and oncogenicity. Rev Med Virol. 2006; 16(2):83-97. Epub 2005 Nov. 16. doi:10.1002/rmv.488. PubMed PMID: 16287204.
64. Nulton T J, Kim N K, DiNardo L J, Morgan I M, Windle B. Patients with integrated HPV16 in head and neck cancer show poor survival. Oral Oncol. 2018; 80:52-5. Epub 2018 May 1. doi: 10.1016/j.oraloncology.2018.03.015. PubMed PMID: 29706188; PMCID: PMC5930384.
65. Veitia D, Liuzzi J, Avila M, Rodriguez I, Toro F, Correnti M. Association of viral load and physical status of HPV-16 with survival of patients with head and neck cancer. Ecancermedicalscience. 2020; 14:1082. Epub 2020 Aug. 31. doi:10.3332/ecancer.2020.1082. PubMed PMID: 32863876; PMCID: PMC7434508.
66. Ang K K, Harris J, Wheeler R, Weber R, Rosenthal D I, Nguyen-Tan P F, Westra W H, Chung C H, Jordan R C, Lu C, Kim H, Axelrod R, Silverman C C, Redmond K P, Gillison M L. Human papillomavirus and survival of patients with oropharyngeal cancer. N Engl J Med. 2010; 363(1):24-35. Epub 2010 Jun. 10. doi: 10.1056/NEJMoa0912217. PubMed PMID: 20530316; PMCID: PMC2943767.
67. Fakhry C, Zhang Q, Nguyen-Tan P F, Rosenthal D I, Weber R S, Lambert L, Trotti A M, 3rd, Barrett W L, Thorstad W L, Jones C U, Yom S S, Wong S J, Ridge J A, Rao SSD, Bonner J A, Vigneault E, Raben D, Kudrimoti M R, Harris J, Le Q T, Gillison M L. Development and Validation of Nomograms Predictive of Overall and Progression-Free Survival in Patients With Oropharyngeal Cancer. J Clin Oncol. 2017; 35(36):4057-65. Epub 2017 Aug. 5. doi: 10.1200/JCO.2016.72.0748. PubMed PMID: 28777690; PMCID: PMC5736236.
68. Keck M K, Zuo Z, Khattri A, Stricker T P, Brown C D, Imanguli M, Rieke D, Endhardt K, Fang P, Bragelmann J, DeBoer R, El-Dinali M, Aktolga S, Lei Z, Tan P, Rozen S G, Salgia R, Weichselbaum R R, Lingen M W, Story M D, Ang K K, Cohen E E, White K P, Vokes E E, Seiwert T Y. Integrative analysis of head and neck cancer identifies two biologically distinct HPV and three non-HPV subtypes. Clin Cancer Res. 2015; 21(4):870-81. Epub 2014 Dec. 11. doi: 10.1158/1078-0432.CCR-14-2481. PubMed PMID: 25492084.
69. Kano M, Kondo S, Wakisaka N, Wakae K, Aga M, Moriyama-Kita M, Ishikawa K, Ueno T, Nakanishi Y, Hatano M, Endo K, Sugimoto H, Kitamura K, Muramatsu M, Yoshizaki T. Expression of estrogen receptor alpha is associated with pathogenesis and prognosis of human papillomavirus-positive oropharyngeal cancer. Int J Cancer. 2019; 145(6):1547-57. Epub 2019 Jun. 23. doi: 10.1002/ijc.32500. PubMed PMID: 31228270.
70. Evans M J, Eckert A, Lai K, Adelman S J, Harnish D C. Reciprocal antagonism between estrogen receptor and N F-kappaB activity in vivo. Circ Res. 2001; 89(9):823-30. Epub 2001 Oct. 27. doi: 10.1161/hh2101.098543. PubMed PMID: 11679413.
71. Zang Y C, Halder J B, Hong J, Rivera V M, Zhang J Z. Regulatory effects of estriol on T cell migration and cytokine profile: inhibition of transcription factor N F-kappa B. J Neuroimmunol. 2002; 124(1-2):106-14. Epub 2002 Apr. 18. doi: 10.1016/s0165-5728(02) 00016-4. PubMed PMID: 11958828.
72. Calippe B, Douin-Echinard V, Laffargue M, Laurell H, Rana-Poussine V, Pipy B, Guery J C, Bayard F, Arnal J F, Gourdy P. Chronic estradiol administration in vivo promotes the proinflammatory response of macrophages to TLR4 activation: involvement of the phosphatidylinositol 3-kinase pathway. J Immunol. 2008; 180(12):7980-8. Epub 2008 Jun. 5. doi: 10.4049/jimmunol. 180. 12.7980. PubMed PMID: 18523261.
73. Hirano S, Furutama D, Hanafusa T. Physiologically high concentrations of 17beta-estradiol enhance N F-kappaB activity in human T cells. Am J Physiol Regul Integr Comp Physiol. 2007; 292(4):R1465-71. Epub 2006 Dec. 30. doi: 10.1152/ajpregu.00778.2006. PubMed PMID: 17194723.
74. Frasor J, El-Shennawy L, Stender J D, Kastrati I. NFkappaB affects estrogen receptor expression and activity in breast cancer through multiple mechanisms. Mol Cell Endocrinol. 2015; 418 Pt 3:235-9. Epub 2014 Dec. 3. doi:10.1016/j.mce.2014.09.013. PubMed PMID: 25450861; PMCID: PMC4402093.
75. Wang C, Huang Y, Sheng J, Huang H, Zhou J. Estrogen receptor alpha inhibits RLR-mediated immune response via ubiquitinating TRAF3. Cell Signal. 2015; 27(10):1977-83. Epub 2015 Jul. 19. doi: 10.1016/j.cellsig.2015.07.008. PubMed PMID: 26186972.
76. Malone E R, Oliva M, Sabatini P J B, Stockley T L, Siu L L. Molecular profiling for precision cancer therapies. Genome Med. 2020; 12(1):8. Epub 2020 Jan. 16. doi: 10.1186/s13073-019-0703-1. PubMed PMID: 31937368; PMCID: PMC6961404.
77. Koenigs M B, Lefranc-Torres A, Bonilla-Velez J, Patel K B, Hayes D N, Glomski K, Busse P M, Chan A W, Clark J R, Deschler D G, Emerick K S, Hammon R J, Wirth L J, Lin D T, Mroz E A, Faquin W C, Rocco J W. Association of Estrogen Receptor Alpha Expression With Survival in Oropharyngeal Cancer Following Chemoradiation Therapy. J Natl Cancer Inst. 2019; 111(9):933-42. Epub 2019 Feb. 5. doi: 10.1093/jnci/djy224. PubMed PMID: 30715409; PMCID: PMC6748818.
78. Parker J S, Mullins M, Cheang M C, Leung S, Voduc D, Vickery T, Davies S, Fauron C, He X, Hu Z, Quackenbush J F, Stijleman I J, Palazzo J, Marron J S, Nobel A B, Mardis E, Nielsen T O, Ellis M J, Perou C M, Bernard P S. Supervised risk predictor of breast cancer based on intrinsic subtypes. J Clin Oncol. 2009; 27(8):1160-7. Epub 2009 Feb. 11. doi: 10.1200/JCO.2008.18.1370. PubMed PMID: 19204204; PMCID: PMC2667820.

### 10. GENERALIZED STATEMENTS OF THE DISCLOSURE

The following numbered statements provide a general description of the disclosure and are not intended to limit the appended claims.

Statement 1: A method for evaluating the prognosis of a human papilloma virus (HPV) associated head and neck cancer patient, comprising detecting defects in nucleic acids encoding genes, or their expression products, for at least five biomarkers selected from the group consisting of TRAF3, CYLD, TRAF2, MYD88, NFKBIA, TNFAIP3, TRAF6, BIRC2, BIRC3, and MAP3K14 in a sample from the patient, normalized against a reference set of nucleic acids encoding genes, or their expression products, in the sample, wherein defects in the nucleic acids or their expression products is indicative of prognosis, thereby evaluating the prognosis of the head and neck cancer patient.

Statement 2: The method of Statement 1, wherein the head and neck cancer is an oropharyngeal squamous cell carcinoma (OPSCC), a nasopharyngeal squamous cell carcinoma, a squamous cell carcinomas of the nasal cavity or paranasal sinuses, a squamous cell carcinoma of the oral cavity, or a squamous cell carcinoma of the hypopharynx.

Statement 3: The method of Statement 2, wherein the head and neck cancer is an oropharyngeal squamous cell carcinoma (OPSCC).

Statement 4: The method of any of Statements 1-3, wherein the presence of defects in the nucleic acids encoding genes, or their expression products, for the biomarkers is indicative of a good prognosis.

Statement 5: The method of any of Statements 1-3, wherein the absence of defects in the nucleic acids encoding genes, or their expression products, for the biomarkers is indicative of a poor prognosis.

Statement 6: The method of any of Statements 1-5, wherein the defects are mutations or copy number alterations.

Statement 7: The method of Statement 6, wherein the mutations are missense mutations, nonsense mutations, frameshift mutations, insertions, and/or deletions.

Statement 8: The method of any of Statements 1-7, wherein the detecting defects in nucleic acids encoding genes, or their expression products, for the biomarkers comprises performing next generation sequencing (NGS), nucleic acid hybridization, quantitative RT-PCR, or immunohistochemistry (IHC), immunocytochemistry (ICC), or immunofluorescence (IF).

Statement 9: The method of any of Statements 1-8, wherein the method for evaluating the prognosis of a head and neck cancer patient further comprises assessment of a medical history, a family history, a physical examination, an endoscopic examination, imaging, a biopsy result, or a combination thereof.

Statement 10: The method of Statement 9, wherein the method is used to develop a treatment strategy for the head and neck cancer patient.

Statement 11: The method of any of Statements 1-10, wherein the nucleic acids encoding genes are isolated from a fixed, paraffin-embedded sample from the patient.

Statement 12: The method of any of Statements 1-11, wherein the nucleic acids encoding genes are isolated from core biopsy tissue or fine needle aspirate cells from the patient.

Statement 13: A method for predicting a response of a human papilloma virus (HPV) associated head and neck cancer patient to a selected treatment, comprising detecting defects in nucleic acids encoding genes, or their expression products, for at least five biomarkers selected from the group consisting of TRAF3, CYLD, TRAF2, MYD88, NFKBIA, TNFAIP3, TRAF6, BIRC2, BIRC3, and MAP3K14 in a sample from the patient, normalized against a reference set of nucleic acids encoding genes, or their expression products, in the sample, wherein defects in the nucleic acids, or their expression products, is indicative of a positive treatment response, thereby predicting the response of the head and cancer patient to the treatment.

Statement 14: The method of Statement 13, wherein the treatment comprises radiation therapy, chemotherapy, immunotherapy, surgery, targeted therapy, or a combination thereof.

Statement 15: A kit comprising at least five nucleic acid probes, wherein each of said probes specifically binds to one of five distinct biomarker nucleic acids or fragments thereof selected from the group consisting of TRAF3, CYLD, TRAF2, MYD88, NFKBIA, TNFAIP3, TRAF6, BIRC2, BIRC3, and MAP3K14.

Statement 16: A method for generating an improved human papilloma virus (HPV) associated head and neck cancer gene expression signature for patient prognosis, the method comprising: (a) training a dataset using TRAF3 and CYLD genomic alteration (mutational or copy number loss) status to identify genes having mRNA expression data associated with NF-kB activity; (b) selecting 10 or more genes with the strongest differential expression found to be associated with NF-kB pathway genomic alteration to be part of a NF-kB activity classifier; and (c) using related mRNA expression levels for the 10 or more genes to generate the improved head and neck cancer gene expression signature for patient prognosis.

Statement 17: The method of Statement 16, wherein 25 or more genes with the strongest prognostic signal are selected.

Statement 18: The method of Statement 16, wherein 50 or more genes with the strongest prognostic signal are selected.

Statement 19: The method of Statement 16, wherein 75 or more genes with the strongest prognostic signal are selected.

Statement 20: A method for evaluating the prognosis of a human papilloma virus (HPV) associated head and neck cancer patient, comprising measuring mRNA expression of at least 10 of the top genes selected from the genes listed of in Table 1 in a sample comprising a cancer cell from the patient, normalized against the expression levels of all RNA transcripts in the sample or a reference set of mRNA expression levels, wherein the mRNA expression levels of the at least 10 genes are indicative of NF-kB activity, thereby evaluating the prognosis of the head and neck cancer patient.

Statement 21: The method of Statement 20, wherein the mRNA expression of 25 or more top genes are measured.

Statement 22: The method of Statement 20, wherein the mRNA expression of 50 or more genes is measured.

Statement 23: The method of any of Statements 20-23, wherein the head and neck cancer is an oropharyngeal squamous cell carcinoma (OPSCC), a nasopharyngeal squamous cell carcinoma, a squamous cell carcinomas of the nasal cavity or paranasal sinuses, a squamous cell carcinoma of the oral cavity, or a squamous cell carcinoma of the hypopharynx.

Statement 24: The method of Statement 23, wherein the head and neck cancer is an an oropharyngeal squamous cell carcinoma (OPSCC).

Statement 25: The method of Statement 1, further comprising detecting defects in a biomarker for ESR1 (estrogen receptor).

Statement 26: The method of Statement 13, further comprising detecting defects in a biomarker for ESR1 (estrogen receptor).

Statement 27: The kit of Statement 15, where the kit further comprises a probe that specifically binds ESR1 or a fragment thereof.

Statement 28: An isolated and purified probe for specifically detecting defects in (a) nucleic acids encoding CYLD mutation N300S or D618A, or (b) their expression products.

Statement 29: The probe of Statement 28, wherein the probe for detecting defects in nucleic acids is a PCR primer or probe.

Statement 30: The probe of Statement 29, wherein the PCR primer is SEQ ID NO. 1, SEQ ID NO. 2, SEQ ID NO. 3, or SEQ ID NO. 4.

Statement 31: The probe of Statement 28, where in the probe specifically detects SEQ ID NO. 6 or SEQ ID NO. 8.

It should be understood that the above description is only representative of illustrative embodiments and examples. For the convenience of the reader, the above description has focused on a limited number of representative examples of all possible embodiments, examples that teach the principles of the disclosure. The description has not attempted to exhaustively enumerate all possible variations or even combinations of those variations described. That alternate embodiments may not have been presented for a specific portion of the disclosure, or that further undescribed alternate embodiments may be available for a portion, is not to be considered a disclaimer of those alternate embodiments. One of ordinary skill will appreciate that many of those undescribed embodiments, involve differences in technology and materials rather than differences in the application of the principles of the disclosure. Accordingly, the disclosure is not intended to be limited to less than the scope set forth in the following claims and equivalents.

Statement Regarding a Nucleotide and/or Amino Acid Sequence Listing

Applicants submit herewith a sequence listing and state that the information recorded in electronic form submitted is identical to the sequence listing as contained in the application as filed. Applicants also state that the computer readable form of the sequence listing is identical to the PDF copy of the sequence listing submitted herewith.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world. It is to be understood that, while the disclosure has been described in conjunction with the detailed description, thereof, the foregoing description is intended to illustrate and not limit the scope. Other aspects, advantages, and modifications are within the scope of the claims set forth below. All publications, patents, and patent applications cited in this specification are herein incorporated by reference as if each individual publication or patent application were specifically and individually indicated to be incorporated by reference.

TABLE 1
Differentially Expressed Genes Used for RNA Classifier Construction. Tumors with
Altered CYLD and/or TRAF3 were compared in terms of RNA expression using RNAseq
data through the TCGA (see Methods section). Top genes by p-value were selected
for classifier construction. The Limma R-project package was used to estimate
the reported fold changes, p-values, t statistics and adjusted p-values.

| Gene | Log fold change | t-statistic | P Value | Adjusted P Value |
|---|---|---|---|---|
| MGAT3\|4248 | 4.72834177 | 13.4636258 | 3.85E−17 | 5.23E−13 |
| STAR\|6770 | 4.34573514 | 12.0733342 | 1.67E−15 | 1.14E−11 |
| VCAM1\|7412 | 4.67998559 | 11.3126361 | 1.46E−14 | 6.61E−11 |
| RAB42\|115273 | 3.16306718 | 10.7910657 | 6.73E−14 | 2.29E−10 |
| NFE2L3\|9603 | 2.30705311 | 10.1885261 | 4.12E−13 | 9.15E−10 |
| FGF2\|2247 | 3.1191258 | 10.2173718 | 3.77E−13 | 9.15E−10 |
| ABCA3\|21 | 4.7253208 | 10.1442421 | 4.71E−13 | 9.15E−10 |
| RNF165\|494470 | 2.88733694 | 9.88705754 | 1.04E−12 | 1.76E−09 |
| PKDCC\|91461 | 4.96888654 | 9.83245056 | 1.23E−12 | 1.85E−09 |
| ZBTB46\|140685 | 2.07965304 | 9.65521619 | 2.12E−12 | 2.89E−09 |
| IL27RA\|9466 | 2.81212246 | 9.58051263 | 2.68E−12 | 3.31E−09 |
| KREMEN2\|79412 | 4.26002249 | 9.50790908 | 3.36E−12 | 3.81E−09 |
| ARNT2\|9915 | 3.67662203 | 9.2276416 | 8.11E−12 | 8.49E−09 |
| MMP19\|4327 | 2.00769653 | 9.0105461 | 1.62E−11 | 1.57E−08 |
| PARM1\|25849 | 3.82774688 | 8.88790558 | 2.39E−11 | 2.17E−08 |
| VRK2\|7444 | 1.43080524 | 8.81420111 | 3.03E−11 | 2.42E−08 |
| COL22A1\|169044 | 4.8141029 | 8.82420642 | 2.93E−11 | 2.42E−08 |
| BIRC3\|330 | 2.85114053 | 8.67277582 | 4.77E−11 | 3.60E−08 |
| SIM2\|6493 | 3.37294181 | 8.57958653 | 6.45E−11 | 4.61E−08 |
| MEGF10\|84466 | 4.80988139 | 8.4680485 | 9.25E−11 | 5.99E−08 |
| MAP3K14\|9020 | 1.84033477 | 8.37716348 | 1.24E−10 | 7.04E−08 |
| C9orf172\|389813 | 2.95800991 | 8.49938468 | 8.36E−11 | 5.68E−08 |
| C11orf92\|399948 | 5.40969838 | 8.38467216 | 1.21E−10 | 7.04E−08 |
| CDH23\|64072 | 3.62130393 | 8.38764937 | 1.20E−10 | 7.04E−08 |
| C8orf42\|157695 | 3.10730116 | 8.25954006 | 1.82E−10 | 9.46E−08 |
| ERO1LB\|56605 | 1.98211825 | 8.23958888 | 1.95E−10 | 9.46E−08 |
| TMEM150C\|441027 | 2.80808814 | 8.24954624 | 1.88E−10 | 9.46E−08 |
| SV2B\|9899 | 4.24669594 | 8.27895568 | 1.71E−10 | 9.31E−08 |
| FAM105B\|90268 | 1.07584613 | 8.13647525 | 2.73E−10 | 1.24E−07 |
| C9orf98\|158067 | 3.28370688 | 8.19619431 | 2.24E−10 | 1.05E−07 |
| CYP27A1\|1593 | 3.40525234 | 8.11863453 | 2.89E−10 | 1.27E−07 |
| LIFR\|3977 | 3.0504013 | 8.10116693 | 3.06E−10 | 1.30E−07 |
| RTN4RL1\|146760 | 3.92520008 | 7.97440421 | 4.65E−10 | 1.86E−07 |
| LOC283174\|283174 | 3.61905068 | 7.99405931 | 4.36E−10 | 1.80E−07 |
| MCF2L\|23263 | 2.17165837 | 7.84251264 | 7.18E−10 | 2.62E−07 |
| NEDD1\|121441 | 1.32523094 | 7.83645923 | 7.33E−10 | 2.62E−07 |
| LOC100272146\|100272146 | 1.44744212 | 7.91509395 | 5.65E−10 | 2.20E−07 |
| TLR6\|10333 | 2.9260823 | 7.85780275 | 6.83E−10 | 2.58E−07 |
| GALNT11\|63917 | 1.42057457 | 7.6552682 | 1.34E−09 | 4.66E−07 |
| CDRT4\|284040 | 1.34725891 | 7.60766801 | 1.56E−09 | 5.23E−07 |
| NT5DC1\|221294 | 1.23072685 | 7.60507358 | 1.58E−09 | 5.23E−07 |
| TRAF2\|7186 | 1.85175494 | 7.5578261 | 1.85E−09 | 5.98E−07 |
| FAM65C\|140876 | 3.19518033 | 7.54885254 | 1.90E−09 | 6.01E−07 |
| ITGAM\|3684 | 2.67120513 | 7.50849655 | 2.18E−09 | 6.72E−07 |
| ZNF488\|118738 | 2.3753282 | 7.47331258 | 2.45E−09 | 7.30E−07 |
| RELB\|5971 | 1.91939244 | 7.47048685 | 2.47E−09 | 7.30E−07 |
| VSTM2L\|128434 | 4.19878746 | 7.44141823 | 2.72E−09 | 7.72E−07 |
| LGI2\|55203 | 4.18695596 | 7.41035964 | 3.02E−09 | 8.37E−07 |
| FAM164A\|51101 | 1.86151097 | 7.39799915 | 3.14E−09 | 8.55E−07 |
| NOXO1\|124056 | 3.16493179 | 7.44101132 | 2.72E−09 | 7.72E−07 |
| CBLN3\|643866 | 2.2116632 | 7.34782971 | 3.72E−09 | 9.91E−07 |
| RNF150\|57484 | 3.59440237 | 7.33072178 | 3.94E−09 | 1.03E−06 |
| C10orf72\|196740 | 3.14111134 | 7.23543136 | 5.41E−09 | 1.37E−06 |
| HVCN1\|84329 | 1.90962335 | 7.23446973 | 5.43E−09 | 1.37E−06 |
| COL4A4\|1286 | 3.76158145 | 7.22194429 | 5.66E−09 | 1.40E−06 |
| CLK4\|57396 | 1.32817903 | 7.18530365 | 6.40E−09 | 1.49E−06 |
| FAM117A\|81558 | 1.54255381 | 7.18220965 | 6.47E−09 | 1.49E−06 |
| RNF19A\|25897 | 1.64110561 | 7.19275059 | 6.25E−09 | 1.49E−06 |
| BCL2\|596 | 2.15300345 | 7.18341174 | 6.44E−09 | 1.49E−06 |
| SPIB\|6689 | 4.5783689 | 7.16490802 | 6.86E−09 | 1.55E−06 |
| TSC22D1\|8848 | 2.14785467 | 7.1259808 | 7.81E−09 | 1.74E−06 |
| SH3BP5\|9467 | 1.94781391 | 7.12193887 | 7.92E−09 | 1.74E−06 |
| NINJ1\|4814 | 1.88131392 | 7.11020752 | 8.24E−09 | 1.78E−06 |
| SYTL3\|94120 | 1.71774437 | 7.07754238 | 9.19E−09 | 1.95E−06 |
| FGF1\|2246 | 2.68618958 | 7.03963382 | 1.04E−08 | 2.15E−06 |
| PKP2\|5318 | 2.77788156 | 7.04508245 | 1.02E−08 | 2.14E−06 |
| RHBDL3\|162494 | 2.83221635 | 7.01515583 | 1.13E−08 | 2.30E−06 |
| GCET2\|257144 | 2.04706548 | 7.00528746 | 1.17E−08 | 2.34E−06 |
| MOXD1\|26002 | 3.26528127 | 6.91237477 | 1.60E−08 | 3.11E−06 |
| GJA3\|2700 | 3.09644469 | 6.89599439 | 1.69E−08 | 3.19E−06 |
| ZMIZ2\|83637 | 1.0444999 | 6.91609061 | 1.58E−08 | 3.11E−06 |
| BTNL9\|153579 | 3.73767575 | 6.87911037 | 1.79E−08 | 3.30E−06 |
| NFKB2\|4791 | 1.49193518 | 6.90008686 | 1.67E−08 | 3.19E−06 |
| TSC2\|7249 | 1.03802414 | 6.87846665 | 1.79E−08 | 3.30E−06 |
| ZNF250\|58500 | 1.17832502 | 6.85218608 | 1.96E−08 | 3.55E−06 |
| PAPLN\|89932 | 2.59341904 | 6.83439193 | 2.08E−08 | 3.72E−06 |
| INPP4A\|3631 | 1.06375794 | 6.77994098 | 2.50E−08 | 4.41E−06 |
| TRAF1\|7185 | 1.70493733 | 6.71505521 | 3.11E−08 | 5.42E−06 |
| LPIN2\|9663 | 1.81819016 | 6.70923026 | 3.17E−08 | 5.46E−06 |
| FAM189A2\|9413 | 3.66919685 | 6.67540484 | 3.56E−08 | 6.04E−06 |
| TPD52L1\|7164 | −1.7446332 | −6.6444954 | 3.95E−08 | 6.62E−06 |
| ADARB2\|105 | 3.58532734 | 6.62709212 | 4.18E−08 | 6.94E−06 |
| NKX2-3\|159296 | 4.08493456 | 6.58277286 | 4.86E−08 | 7.96E−06 |
| RASD2\|23551 | 3.18335212 | 6.56849196 | 5.10E−08 | 8.16E−06 |
| ING1\|3621 | 1.48204371 | 6.56827265 | 5.10E−08 | 8.16E−06 |
| WNT10B\|7480 | 2.52362561 | 6.55590603 | 5.32E−08 | 8.41E−06 |
| GORAB\|92344 | 0.86334783 | 6.53209714 | 5.76E−08 | 9.01E−06 |
| HOXB13\|10481 | 4.59980368 | 6.50858446 | 6.24E−08 | 9.64E−06 |
| PRODH\|5625 | 2.36027265 | 6.50186891 | 6.38E−08 | 9.75E−06 |
| CD8B\|926 | 2.65757458 | 6.46214885 | 7.30E−08 | 1.10E−05 |
| RANBP17\|64901 | 2.32682556 | 6.45538596 | 7.47E−08 | 1.12E−05 |
| CEP135\|9662 | 1.1005867 | 6.44824441 | 7.65E−08 | 1.13E−05 |
| FUCA2\|2519 | −1.0207717 | −6.4243012 | 8.29E−08 | 1.21E−05 |
| SLC12A7\|10723 | 2.36316554 | 6.41734387 | 8.49E−08 | 1.22E−05 |
| PPFIBP2\|8495 | 1.33610993 | 6.41591164 | 8.53E−08 | 1.22E−05 |
| ZDHHC9\|51114 | −1.177593 | −6.3970044 | 9.09E−08 | 1.29E−05 |
| ICOSLG\|23308 | 2.01347976 | 6.38449654 | 9.49E−08 | 1.33E−05 |
| PLD6\|201164 | 1.68765203 | 6.35648775 | 1.04E−07 | 1.43E−05 |
| GGA2\|23062 | 1.24474257 | 6.3776102 | 9.71E−08 | 1.35E−05 |
| SCNN1G\|6340 | 3.23550356 | 6.33083308 | 1.14E−07 | 1.53E−05 |
| ARHGAP26\|23092 | 1.77082328 | 6.33230117 | 1.13E−07 | 1.53E−05 |
| ATL2\|64225 | 1.22582634 | 6.31641414 | 1.19E−07 | 1.59E−05 |
| CDC42EP4\|23580 | 1.83506519 | 6.30414341 | 1.24E−07 | 1.63E−05 |
| SCD5\|79966 | 1.36558878 | 6.31068113 | 1.22E−07 | 1.61E−05 |
| TLR1\|7096 | 2.19107035 | 6.27888919 | 1.36E−07 | 1.75E−05 |
| ARHGAP28\|79822 | 2.96803442 | 6.24946341 | 1.50E−07 | 1.88E−05 |
| BBS1\|582 | 0.80989877 | 6.261124 | 1.44E−07 | 1.85E−05 |
| SH2B3\|10019 | 1.40311454 | 6.25786294 | 1.45E−07 | 1.85E−05 |
| STXBP1\|6812 | 2.04352973 | 6.23661508 | 1.56E−07 | 1.95E−05 |
| LARP6\|55323 | 1.74516494 | 6.2104996 | 1.71E−07 | 2.11E−05 |
| FRMD4A\|55691 | 1.74353166 | 6.20209856 | 1.76E−07 | 2.15E−05 |
| AMPD3\|272 | 1.46582728 | 6.19279917 | 1.81E−07 | 2.20E−05 |
| DHCR24\|1718 | −1.3342424 | −6.1728925 | 1.94E−07 | 2.33E−05 |
| JAZF1\|221895 | 1.27844665 | 6.10003149 | 2.48E−07 | 2.90E−05 |
| PRR5L\|79899 | 1.74068243 | 6.1076165 | 2.42E−07 | 2.86E−05 |
| UBD\|10537 | 3.22005619 | 6.1206842 | 2.31E−07 | 2.76E−05 |
| KSR1\|8844 | 1.09772952 | 6.09714481 | 2.50E−07 | 2.91E−05 |
| EPHB1\|2047 | 2.97964534 | 6.03169934 | 3.12E−07 | 3.60E−05 |
| SLC12A8\|84561 | −2.6585792 | −6.0207018 | 3.24E−07 | 3.64E−05 |
| NCALD\|83988 | 1.91489908 | 6.02147464 | 3.23E−07 | 3.64E−05 |
| B4GALT6\|9331 | 1.56121823 | 5.99839654 | 3.49E−07 | 3.86E−05 |
| QDPR\|5860 | 1.38477889 | 6.00947835 | 3.36E−07 | 3.75E−05 |
| PNRC1\|10957 | 1.18502462 | 6.0209941 | 3.23E−07 | 3.64E−05 |
| IL18R1\|8809 | 1.53807793 | 5.96870651 | 3.86E−07 | 4.16E−05 |
| NMT2\|9397 | 1.29761403 | 5.98860377 | 3.61E−07 | 3.92E−05 |
| CD207\|50489 | 2.9871545 | 5.96076075 | 3.96E−07 | 4.18E−05 |
| SERPINF2\|5345 | 1.69006376 | 5.96277006 | 3.94E−07 | 4.18E−05 |
| IL2RG\|3561 | 2.35564092 | 5.99377439 | 3.55E−07 | 3.89E−05 |
| RAB36\|9609 | 1.75928334 | 5.94398552 | 4.19E−07 | 4.39E−05 |
| ECE1\|1889 | 1.55975574 | 5.96261113 | 3.94E−07 | 4.18E−05 |
| C1orf21\|81563 | −1.5291274 | −5.9332394 | 4.35E−07 | 4.51E−05 |
| KIAA1908\|114796 | 1.16659767 | 5.91436042 | 4.63E−07 | 4.74E−05 |
| MTMR7\|9108 | 1.61041802 | 5.89668918 | 4.92E−07 | 4.99E−05 |
| MMP28\|79148 | 3.39579421 | 5.91773994 | 4.58E−07 | 4.72E−05 |
| TNFRSF9\|3604 | 2.00164437 | 5.83369667 | 6.08E−07 | 5.99E−05 |
| DNAJB11\|51726 | −1.0301446 | −5.8815626 | 5.18E−07 | 5.21E−05 |
| FOXN1\|8456 | 2.69962123 | 5.82415599 | 6.28E−07 | 6.14E−05 |
| FXYD6\|53826 | 2.45902462 | 5.82156736 | 6.33E−07 | 6.15E−05 |
| RNF44\|22838 | 1.06076882 | 5.84558303 | 5.84E−07 | 5.84E−05 |
| ORAI2\|80228 | 1.41140169 | 5.83578387 | 6.04E−07 | 5.99E−05 |
| C12orf34\|84915 | 1.53587859 | 5.79810439 | 6.85E−07 | 6.61E−05 |
| CLIP3\|25999 | 2.68227374 | 5.76920503 | 7.55E−07 | 7.18E−05 |
| FAM171A1\|221061 | 2.10182171 | 5.78576158 | 7.14E−07 | 6.84E−05 |
| FAM161A\|84140 | 1.13846936 | 5.735224 | 8.47E−07 | 7.73E−05 |
| C11orf41\|25758 | 2.48021109 | 5.7181002 | 8.97E−07 | 8.02E−05 |
| ABCC4\|10257 | 1.58369548 | 5.73885419 | 8.36E−07 | 7.73E−05 |
| TMC8\|147138 | 1.95291468 | 5.74879083 | 8.09E−07 | 7.59E−05 |
| C6orf105\|84830 | 2.6046787 | 5.68869121 | 9.90E−07 | 8.74E−05 |
| ARPC1A\|10552 | −0.8289561 | −5.7501981 | 8.05E−07 | 7.59E−05 |
| C7orf44\|55744 | 0.7624916 | 5.73607548 | 8.44E−07 | 7.73E−05 |

TABLE 2
Genes in the final NF-kB classifier. Log Fold-Change and
Adjusted P-Values were generated with LIMMA, comparing
differential expression of classifier genes when comparing
of true-positives and true-negatives cases based on the
initial (unimproved) classifier, see Methods.

| HUGO Gene Name | Log Fold-Change | Adjusted P-Value |
|---|---|---|
| MGAT3 | 4.72834177 | 5.23E−13 |
| STAR | 4.345735136 | 1.14E−11 |
| VCAM1 | 4.679985591 | 6.61E−11 |
| RAB42 | 3.163067177 | 2.29E−10 |
| NFE2L3 | 2.307053108 | 9.15E−10 |
| FGF2 | 3.119125796 | 9.15E−10 |
| ABCA3 | 4.725320799 | 9.15E−10 |
| RNF165 | 2.887336939 | 1.76E−09 |
| PKDCC | 4.968886543 | 1.85E−09 |
| ZBTB46 | 2.079653042 | 2.89E−09 |
| IL27RA | 2.812122457 | 3.31E−09 |
| KREMEN2 | 4.260022489 | 3.81E−09 |
| ARNT2 | 3.676622025 | 8.49E−09 |
| MMP19 | 2.00769653 | 1.57E−08 |
| PARM1 | 3.827746878 | 2.17E−08 |
| VRK2 | 1.430805242 | 2.42E−08 |
| COL22A1 | 4.814102899 | 2.42E−08 |
| BIRC3 | 2.851140525 | 3.60E−08 |
| SIM2 | 3.372941806 | 4.61E−08 |
| MEGF10 | 4.809881389 | 5.99E−08 |
| MAP3K14 | 1.840334773 | 7.04E−08 |
| C9orf172 | 2.958009915 | 5.68E−08 |
| C11orf92 | 5.409698384 | 7.04E−08 |
| CDH23 | 3.621303931 | 7.04E−08 |
| C8orf42 | 3.107301157 | 9.46E−08 |
| ERO1LB | 1.982118254 | 9.46E−08 |
| TMEM150C | 2.808088143 | 9.46E−08 |
| SV2B | 4.246695942 | 9.31E−08 |
| FAM105B | 1.075846134 | 1.24E−07 |
| C9orf98 | 3.28370688 | 1.05E−07 |
| CYP27A1 | 3.40525234 | 1.27E−07 |
| LIFR | 3.050401304 | 1.30E−07 |
| RTN4RL1 | 3.925200083 | 1.86E−07 |
| LOC283174 | 3.619050677 | 1.80E−07 |
| MCF2L | 2.171658374 | 2.62E−07 |
| NEDD1 | 1.325230936 | 2.62E−07 |

TABLE 3
Sets of highly autocorrelated genes after weighted gene correlation network analysis (WGCNA).

| | | | | | | | | | | | |
|---|---|---|---|---|---|---|---|---|---|---|---|
| WG | Hugo | BR | ACVRL1 | GN | AEN | BR | ALDH7A1 | YE | ANKRD29 | BR | APLNR |
| BL | A2LD1 | RE | ACYP1 | GN | AES | BL | ALDH9A1 | RE | ANKRD36 | BL | APLN |
| MA | A2ML1 | GY | ADAL | GN | AFAP1L1 | GN | ALDOA | GY | ANKRD37 | PI | APOB48R |
| BR | A2M | BR | ADAM12 | YE | AFAP1L2 | GN | ALG1 | MA | ANKRD56 | BR | APOBEC3B |
| YE | AACS | MA | ADAM15 | RE | AFG3L1 | GY | ALG2 | RE | ANKS3 | BL | APOBEC3D |
| YE | ABCA17P | YE | ADAM19 | GY | AFG3L2 | BR | ALG6 | GY | ANKS6 | BL | APOBEC3F |
| YE | ABCA3 | BR | ADAM23 | BR | AG2 | GY | ALG8 | RE | ANKZF1 | BL | APOBEC3G |
| BL | ABCA7 | BL | ADAM28 | BL | AGAP2 | BR | ALKBH1 | YE | ANO4 | PI | APOC1 |
| YE | ABCC4 | BL | ADAM6 | RE | AGAP4 | GN | ALKBH2 | GY | ANO8 | PI | APOC2 |
| BR | ABCC9 | YE | ADAM8 | RE | AGAP6 | GY | ALKBH5 | BR | ANPEP | BR | APOD |
| PI | ABCD1 | BL | ADAMDEC1 | GN | AGA | GN | ALKBH7 | BR | ANTXR2 | PI | APOE |
| GY | ABCF2 | BR | ADAMTS12 | BL | AGBL5 | BL | ALOX12 | MA | ANXA1 | PI | APOL4 |
| BR | ABCG1 | BR | ADAMTS14 | RE | AGER | PI | ALOX15B | MA | ANXA2P1 | BR | APOLD1 |
| BR | ABHD3 | YE | ADAMTS17 | GY | AGMAT | PI | ALOX5AP | MA | ANXA2P2 | GN | APTX |
| GY | ABHD4 | BR | ADAMTS2 | YE | AGPAT3 | PI | ALOX5 | MA | ANXA2P3 | BR | AQP1 |
| MA | ABI2 | BR | ADAMTS4 | MA | AGPAT4 | GN | ALPK1 | MA | ANXA2 | MA | AQP3 |
| BL | ABI3BP | BR | ADAMTS7 | GY | AGR2 | YE | ALPK2 | BL | ANXA3 | BL | AQP5 |
| BL | ABI3 | BR | ADAMTS9 | RE | AHSA2 | BR | ALPL | GY | ANXA4 | PI | ARAP1 |
| GY | ABLIM3 | BR | ADAMTSL2 | GY | AIF1L | PI | ALS2CR4 | BR | ANXA5 | BL | ARAP3 |
| BR | ABP1 | BL | ADAMTSL5 | PI | AIF1 | GY | ALX3 | BL | ANXA6 | GN | ARF3 |
| YE | ABTB2 | PI | ADAP2 | BL | AIG1 | BL | AMACR | GY | ANXA8L2 | GY | ARG2 |
| BR | ACAA2 | BL | ADARB1 | MA | AIM1L | BL | AMICA1 | GY | ANXA8 | RE | ARGLU1 |
| GY | ACACB | YE | ADARB2 | YE | AK3L1 | BR | AMIGO2 | PI | AOAH | BL | ARHGAP15 |
| RE | ACAD11 | RE | ADAT2 | BR | AKAP12 | GY | AMN1 | BR | AOC3 | PI | ARHGAP18 |
| BL | ACAP1 | GN | ADAT3 | BL | AKAP5 | BR | AMOT | BR | AOX1 | YE | ARHGAP22 |
| BR | ACAT2 | GN | ADCK2 | BL | AKAP7 | BR | AMPD2 | RE | AP1B1 | MA | ARHGAP23 |
| BR | ACBD7 | BR | ADCY1 | BR | AKAP8 | YE | AMPD3 | GN | AP1M1 | BL | ARHGAP25 |
| BL | ACCN2 | BR | ADCY4 | GY | AKIRIN2 | YE | AMTN | YE | AP1M2 | YE | ARHGAP26 |
| GN | ACD | BR | ADCY5 | BL | AKNA | RE | AMT | PI | AP1S2 | MA | ARHGAP27 |
| PI | ACE | GN | ADCY6 | MA | AKR1B10 | RE | AMY2B | GY | AP2A1 | BR | ARHGAP28 |
| BR | ACIN1 | YE | ADC | YE | AKR1C1 | GN | AMZ2 | BR | AP2B1 | BL | ARHGAP30 |
| GN | ACO2 | GY | ADH5 | YE | AKR1C2 | GN | ANAPC7 | GY | AP3B2 | YE | ARHGAP31 |
| MA | ACOT11 | GY | ADH7 | YE | AKR1C3 | BR | ANGPT2 | GN | AP3D1 | RE | ARHGAP33 |
| PI | ACP2 | BL | ADM | GY | AKT2 | BR | ANGPTL2 | MA | AP3M2 | BL | ARHGAP9 |
| PI | ACP5 | BL | ADORA2A | GY | ALDH1A1 | GY | ANGPTL4 | GY | AP3S2 | BL | ARHGDIB |
| BR | ACSL1 | GY | ADORA2B | BR | ALDH1B1 | GN | ANK1 | BR | APBA2 | MA | ARHGEF10L |
| BR | ACTA2 | PI | ADORA3 | BR | ALDH1L2 | BR | ANK2 | GN | APBA3 | BR | ARHGEF15 |
| PI | ACTB | GY | ADO | YE | ALDH2 | BL | ANKDD1A | BL | APBB1IP | BR | ARHGEF16 |
| BL | ACTG1 | BL | ADPGK | GY | ALDH3A1 | YE | ANKH | BR | APBB2 | BR | ARHGEF17 |
| BR | ACTG2 | BL | ADPRH | GY | ALDH3A2 | YE | ANKLE2 | RE | APBB3 | GN | ARHGEF18 |
| BR | ACTN1 | BL | ADRA2A | PI | ALDH3B1 | RE | ANKMY1 | BR | APCDD1 | BL | ARHGEF1 |
| BR | ACTR6 | BR | ADRB2 | MA | ALDH3B2 | PI | ANKMY2 | BL | APH1A | BR | ARHGEF2 |
| RE | ACVR1 | BL | ADRBK2 | GY | ALDH4A1 | MA | ANKRD13B | PI | APH1B | MA | ARHGEF37 |
| BL | ACVR2A | BR | AEBP1 | BL | ALDH5A1 | GN | ANKRD16 | GN | APLF | MA | ARHGEF4 |
| BL | ARHGEF6 | BR | ATL1 | BL | BANK1 | BL | BMPR1B | BL | C12orf26 | BL | C19orf21 |
| BL | ARID5A | YE | ATOH8 | GY | BARX1 | GY | BMS1 | YE | C12orf34 | GN | C19orf22 |
| BR | ARL4C | BR | ATP10A | MA | BARX2 | BR | BNC2 | MA | C12orf41 | GN | C19orf24 |
| YE | ARL4D | MA | ATP10B | BL | BASP1 | MA | BNIPL | BR | C12orf56 | GN | C19orf25 |
| BL | ARL6IP5 | GN | ATP13A1 | BL | BATF | BR | BOC | GN | C12orf5 | GN | C19orf28 |
| MA | ARL8B | YE | ATP13A2 | GN | BBS12 | MA | BPNT1 | RE | C12orf76 | GN | C19orf29 |
| GN | ARMC6 | MA | ATP13A4 | GN | BBS4 | GN | BRMS1L | PI | C13orf15 | MA | C19orf33 |
| BR | ARMC9 | GY | ATP1A1 | GY | BBS5 | GN | BSG | BL | C13orf18 | RE | C19orf36 |
| BR | ARMCX1 | BL | ATP1B1 | GN | BBS7 | BL | BTBD10 | GY | C13orf1 | BR | C19orf40 |
| YE | ARNT2 | YE | ATP1B3 | GN | BBS9 | MA | BTBD11 | BR | C13orf29 | GN | C19orf43 |
| YE | ARPC1A | BL | ATP2A3 | MA | BCAS1 | GN | BTBD2 | GN | C13orf31 | RE | C19orf44 |
| YE | ARRB1 | YE | ATP2C2 | BR | BCAT1 | GY | BTD | BR | C13orf33 | GN | C19orf50 |
| PI | ARRB2 | GN | ATP5A1 | GN | BCKDK | YE | BTF3L4 | MA | C14orf129 | GN | C19orf52 |
| GN | ARSB | GN | ATP5B | MA | BCL10 | BL | BTG1 | GN | C14orf132 | GN | C19orf53 |
| GN | ARSD | GN | ATP5D | BL | BCL11A | BR | BTG3 | BL | C14orf139 | GN | C19orf54 |
| GY | ARSI | GN | ATP5SL | BL | BCL11B | BL | BTK | YE | C14orf147 | GN | C19orf56 |
| PI | ASAH1 | YE | ATP6AP2 | BL | BCL2A1 | YE | BTNL9 | BR | C14orf169 | GN | C19orf57 |
| BR | ASAP3 | BL | ATP6V1B2 | GN | BCL2L12 | BR | BUB3 | YE | C14orf73 | GN | C19orf60 |
| BR | ASB1 | BL | ATP8A1 | RE | BCL2L13 | GN | BVES | BR | C15orf23 | GN | C19orf62 |
| GY | ASB2 | BL | ATP8B2 | BL | BCL2L14 | RE | BZRAP1 | YE | C15orf29 | GN | C19orf6 |
| RE | ASB6 | BR | ATPBD4 | GY | BCL2L2 | GY | BZW2 | MA | C15orf39 | GN | C19orf70 |
| GY | ASB8 | BL | ATXN10 | YE | BCL2 | YE | C10orf10 | GY | C15orf44 | PI | C1QA |
| YE | ASB9 | RE | ATXN7L2 | GY | BCL3 | BR | C10orf137 | BL | C15orf57 | GN | C1QBP |
| GY | ASCC1 | BR | AUH | BR | BCL6B | BR | C10orf26 | BR | C16orf45 | PI | C1QB |
| GY | ASF1A | BR | AURKA | MA | BCL7A | BL | C10orf54 | BL | C16orf54 | PI | C1QC |
| BR | ASF1B | BR | AURKB | GN | BCL9L | MA | C10orf57 | GY | C16orf73 | YE | C1QTNF1 |
| GN | ASNA1 | GN | AXIN1 | BL | BCR | YE | C10orf72 | BL | C16orf74 | BR | C1QTNF3 |
| GY | ASNSD1 | BR | AXIN2 | YE | BDH1 | BR | C10orf78 | BR | C16orf75 | BR | C1QTNF6 |
| BR | ASPN | BR | AXL | MA | BDKRB2 | GY | C10orf81 | BR | C17orf28 | GN | C1RL |
| BR | ASRGL1 | GY | B3GALTL | YE | BECN1 | GN | C10orf88 | GY | C17orf51 | BR | C1R |
| BR | ASTE1 | GY | B3GNT3 | BR | BEX2 | MA | C10orf99 | BR | C17orf53 | BR | C1S |
| YE | ASTN2 | MA | B3GNT7 | BR | BGN | YE | C11orf41 | RE | C17orf56 | MA | C1orf106 |
| GY | ATAD1 | MA | B3GNT8 | YE | BHLHE41 | MA | C11orf46 | GY | C17orf58 | RE | C1orf113 |
| RE | ATAD3B | BL | B3GNT9 | BL | BIK | GY | C11orf54 | RE | C17orf65 | MA | C1orf116 |
| YE | ATF5 | GY | B4GALNT1 | BL | BIN2 | BR | C11orf57 | BL | C17orf68 | MA | C1orf126 |
| BL | ATF7IP2 | BL | B4GALNT4 | YE | BIRC3 | YE | C11orf58 | RE | C17orf86 | BR | C1orf131 |
| GY | ATG16L1 | BR | B4GALT1 | BL | BLK | RE | C11orf61 | GN | C17orf97 | BR | C1orf135 |
| RE | ATG16L2 | YE | B4GALT3 | BL | BLNK | GN | C11orf84 | GY | C18orf10 | GY | C1orf144 |
| GY | ATG2A | YE | B4GALT6 | BR | BMF | YE | C11orf92 | YE | C18orf1 | PI | C1orf162 |
| GN | ATG4D | BR | BACE1 | BR | BMP1 | YE | C11orf93 | GN | C18orf55 | MA | C1orf170 |
| GY | ATG5 | BL | BACE2 | YE | BMP2 | BR | C11orf95 | GN | C18orf8 | BR | C1orf172 |
| GY | ATG9A | YE | BAI2 | BR | BMP6 | BL | C11orf9 | GN | C19orf10 | BR | C1orf174 |
| RE | ATG9B | BL | BAIAP2L1 | GY | BMP7 | GN | C12orf10 | PI | C19orf12 | RE | C1orf175 |
| RE | ATHL1 | BL | BAIAP2 | BR | BMP8A | YE | C12orf23 | GN | C19orf20 | BR | C1orf198 |
| YE | C1orf201 | GY | C4orf43 | BR | C9orf150 | GN | CARM1 | GN | CCDC86 | PI | CD209 |
| MA | C1orf210 | BL | C4orf7 | GY | C9orf21 | GY | CASP3 | BL | CCDC88B | BL | CD22 |
| YE | C1orf21 | PI | C5AR1 | GY | C9orf25 | BL | CASP6 | YE | CCDC8 | BL | CD247 |
| BL | C1orf226 | BR | C5orf13 | BL | C9orf30 | GN | CASP8 | GY | CCDC90B | BR | CD248 |
| PI | C1orf38 | BR | C5orf15 | GN | C9orf40 | BR | CASP9 | GN | CCDC94 | MA | CD24 |
| PI | C1orf54 | BL | C5orf20 | RE | C9orf45 | BR | CAT | YE | CCDC97 | BL | CD274 |
| RE | C1orf63 | GY | C5orf23 | YE | C9orf85 | GN | CAV1 | GN | CCDC9 | BR | CD276 |
| BL | C1orf74 | RE | C5orf34 | BL | C9orf91 | GN | CAV2 | PI | CCL18 | BL | CD27 |
| YE | C1orf93 | BR | C5orf35 | YE | C9orf98 | BL | CBARA1 | BL | CCL19 | BL | CD28 |
| MA | C20orf108 | BL | C5orf39 | MA | CA12 | BR | CBFA2T3 | YE | CCL20 | BL | CD2 |
| YE | C20orf112 | BL | C5orf53 | YE | CA2 | BL | CBLC | BL | CCL21 | PI | CD300A |
| YE | C20orf54 | GY | C5orf54 | GY | CA9 | YE | CBLN2 | BL | CCL22 | PI | CD300LF |
| BR | C21orf45 | BL | C5orf56 | BR | CAB39L | YE | CBLN3 | PI | CCL2 | YE | CD302 |
| GY | C21orf56 | BR | C5orf62 | BR | CABLES2 | GN | CBR4 | PI | CCL3 | GN | CD320 |
| RE | C21orf58 | YE | C6orf105 | GY | CACNA1B | BR | CBS | BL | CCL4L2 | BR | CD34 |
| GY | C22orf13 | MA | C6orf132 | BR | CACNA1C | BR | CBWD6 | BL | CCL4 | BR | CD36 |
| GY | C22orf23 | RE | C6orf134 | BR | CACNA1H | BL | CBX1 | BL | CCL5 | BL | CD37 |
| YE | C22orf28 | YE | C6orf141 | BR | CADM1 | BR | CBX2 | BR | CCNB1 | BL | CD38 |
| BR | C22orf46 | GN | C6orf162 | BR | CADM3 | GY | CBX4 | BR | CCNB2 | BL | CD3D |
| BL | C2CD2L | YE | C6orf168 | BL | CADM4 | YE | CBX7 | YE | CCND1 | BL | CD3E |
| YE | C2CD2 | GY | C6orf182 | YE | CADPS2 | GN | CC2D1A | BL | CCND2 | BL | CD3G |
| GN | C2CD4B | BL | C6orf223 | YE | CALB1 | BL | CC2D2A | GY | CCNDBP1 | BL | CD40 |
| MA | C2orf29 | BL | C6orf64 | BR | CALCRL | GY | CCBL2 | BR | CCNF | BR | CD47 |
| BL | C2orf43 | GY | C7orf25 | BR | CALD1 | GN | CCDC111 | BL | CCNG1 | BL | CD48 |
| MA | C2orf55 | GY | C7orf28B | BL | CALHM2 | MA | CCDC120 | GN | CCNG2 | PI | CD4 |
| RE | C2orf56 | BL | C7orf29 | MA | CALML3 | GN | CCDC123 | YE | CCNJL | BL | CD52 |
| GY | C2orf65 | BL | C7orf31 | RE | CALML4 | GN | CCDC124 | RE | CCNL2 | BL | CD53 |
| GN | C2orf67 | BR | C7orf42 | BR | CALU | BR | CCDC125 | PI | CCR1 | BL | CD55 |
| BR | C2orf77 | YE | C7orf44 | PI | CAMK1 | RE | CCDC130 | BL | CCR2 | YE | CD59 |
| GN | C2orf79 | BR | C7orf46 | GN | CAMK2D | GY | CCDC134 | BL | CCR4 | BL | CD5 |
| PI | C2 | YE | C7orf49 | BR | CAMK2N1 | YE | CCDC149 | BL | CCR5 | PI | CD68 |
| PI | C3AR1 | BR | C7orf58 | RE | CANT1 | RE | CCDC150 | BL | CCR6 | BL | CD69 |
| GY | C3orf14 | BL | C7orf68 | RE | CAPN10 | GY | CCDC25 | BL | CCR7 | BL | CD6 |
| BL | C3orf52 | GY | C7orf70 | MA | CAPN14 | YE | CCDC28B | GN | CCT5 | BL | CD72 |
| BL | C3orf57 | BL | C7 | BL | CAPN1 | YE | CCDC3 | BL | CD101 | BL | CD74 |
| BL | C3orf59 | GN | C8orf38 | MA | CAPN2 | BL | CCDC43 | PI | CD14 | BL | CD79A |
| GN | C3orf64 | GN | C8orf41 | MA | CAPN5 | RE | CCDC45 | PI | CD163 | BL | CD79B |
| BL | C3 | YE | C8orf42 | RE | CAPRIN2 | RE | CCDC57 | GY | CD177 | BL | CD7 |
| BR | C4A | YE | C8orf4 | GY | CARD10 | MA | CCDC64B | BL | CD180 | PI | CD81 |
| YE | C4orf14 | MA | C8orf73 | BL | CARD11 | BR | CCDC64 | BL | CD19 | GY | CD82 |
| MA | C4orf19 | GY | C8orf79 | MA | CARD14 | GN | CCDC68 | BL | CD1A | BL | CD83 |
| GY | C4orf33 | BR | C9orf100 | PI | CARD16 | BL | CCDC69 | BL | CD1E | BL | CD84 |
| GN | C4orf34 | BL | C9orf125 | BL | CARD8 | GY | CCDC77 | YE | CD200 | PI | CD86 |
| GN | C4orf41 | BR | C9orf140 | BL | CARD9 | BR | CCDC80 | BL | CD207 | BL | CD8A |
| BL | CD8B | GY | CDS2 | BR | CHPF | YE | CLIP3 | YE | COL23A1 | BR | CPXM2 |
| BR | CD93 | GN | CDT1 | YE | CHPT1 | GN | CLIP4 | GN | COL27A1 | BR | CPZ |
| BL | CD96 | MA | CEACAM1 | BR | CHRDL1 | RE | CLK1 | BR | COL3A1 | BL | CR1 |
| BL | CD97 | MA | CEACAM5 | BR | CHRD | RE | CLK2 | BR | COL4A1 | BL | CR2 |
| MA | CD99L2 | MA | CEACAM6 | GN | CHST10 | YE | CLN5 | BR | COL4A2 | GY | CRAT |
| BR | CDAN1 | MA | CEACAM7 | PI | CHST11 | GY | CLN8 | YE | COL4A4 | YE | CRB2 |
| BL | CDC16 | GN | CEBPD | YE | CHST14 | GY | CLNS1A | BR | COL5A1 | GN | CRB3 |
| GN | CDC34 | BR | CEBPG | YE | CHST15 | BR | CLP1 | BR | COL5A2 | GY | CRBN |
| GN | CDC37 | PI | CECR1 | BR | CHST1 | GN | CLPP | BR | COL5A3 | BL | CRCP |
| PI | CDC42BPG | GN | CECR5 | BL | CHST2 | BL | CLSTN3 | BR | COL6A1 | YE | CREB3L1 |
| BR | CDC42EP3 | BL | CELF2 | BL | CHST6 | GN | CLTA | BR | COL6A2 | GN | CREB5 |
| YE | CDC42EP4 | BL | CEL | GN | CHST7 | BR | CLU | BR | COL6A3 | BL | CREBL2 |
| BR | CDC42EP5 | BR | CENPA | RE | CHTF18 | BR | CMAH | BR | COL8A1 | PI | CREG1 |
| BL | CDC42SE2 | BR | CENPQ | BR | CIDEB | GY | CMAS | BR | COLEC12 | YE | CREM |
| MA | CDC42 | RE | CENPT | BL | CIITA | GY | CMBL | YE | COMMD10 | BL | CRISPLD1 |
| BR | CDCA5 | GN | CENPV | BR | CILP2 | PI | CMKLR1 | BR | COMP | BR | CRISPLD2 |
| PI | CDCA7L | YE | CEP135 | BL | CISH | PI | CMTM3 | GN | COPE | GY | CRMP1 |
| BR | CDH11 | GY | CEP250 | BL | CITED2 | YE | CMTM4 | BR | COPS3 | RE | CROCCL1 |
| BR | CDH13 | BR | CEP72 | YE | CIZ1 | BL | CMTM7 | GN | COPS5 | GN | CROCC |
| YE | CDH23 | BR | CERCAM | BR | CKAP4 | PI | CNDP2 | BL | COPS7A | BL | CRTAM |
| MA | CDH26 | BR | CERK | YE | CKMT1B | BR | CNN1 | GN | COQ5 | GN | CRTC1 |
| GY | CDH3 | BR | CES3 | MA | CLCA2 | GN | CNN2 | BR | COQ7 | BR | CRY2 |
| BR | CDH5 | GN | CFD | MA | CLCA4 | GN | CNN3 | BL | CORO1A | GN | CRYZ |
| GY | CDHR1 | BR | CFI | BL | CLCF1 | GY | CNNM2 | BL | CORO7 | RE | CSAD |
| GN | CDIPT | YE | CFLAR | BR | CLCN4 | GN | CNOT3 | BL | COTL1 | PI | CSF1R |
| RE | CDK10 | BL | CFP | BL | CLCN6 | BR | CNOT8 | GY | COX10 | BL | CSF1 |
| BR | CDK11A | YE | CGNL1 | YE | CLDN10 | BR | CNRIP1 | GN | COX11 | BL | CSF2RA |
| BL | CDK16 | MA | CGN | BL | CLDN15 | BR | CNTD1 | GY | COX15 | BL | CSF2RB |
| YE | CDK18 | YE | CGRRF1 | MA | CLDN23 | YE | CNTNAP2 | GN | COX4I1 | PI | CSF3R |
| BR | CDK1 | BR | CH25H | YE | CLDN3 | BR | CNTROB | GN | COX5A | BR | CSGALNACT1 |
| RE | CDK3 | YE | CHAC2 | MA | CLDN4 | GY | COCH | YE | COX6B2 | GN | CSGALNACT2 |
| GN | CDK4 | BR | CHAF1A | BR | CLDN7 | GN | COG3 | BR | CPA3 | BL | CSK |
| GY | CDK5RAP2 | GN | CHCHD3 | BL | CLEC10A | GY | COG7 | YE | CPAMD8 | GN | CSNK1D |
| RE | CDK5RAP3 | GY | CHDH | BR | CLEC11A | BR | COL10A1 | BL | CPEB1 | BL | CSNK1E |
| MA | CDKN1A | PI | CHEK1 | BR | CLEC14A | BR | COL11A1 | GN | CPEB2 | GN | CSNK1G2 |
| BL | CDKN1B | PI | CHI3L1 | YE | CLEC1A | BR | COL12A1 | GN | CPE | BR | CSPG4 |
| BL | CDKN1C | BL | CHI3L2 | BL | CLEC2D | BR | COL14A1 | GN | CPM | BR | CST1 |
| BR | CDKN2A | PI | CHIT1 | BR | CLEC3B | BR | COL15A1 | YE | CPNE2 | BL | CST7 |
| MA | CDKN2B | RE | CHKB.CPT1B | PI | CLEC5A | YE | COL16A1 | BL | CPNE5 | MA | CSTB |
| BR | CDKN2C | PI | CHMP4C | PI | CLEC7A | YE | COL18A1 | PI | CPNE7 | BL | CTBP2 |
| YE | CDON | BL | CHMP7 | YE | CLGN | YE | COL19A1 | RE | CPT1B | GN | CTDP1 |
| BL | CDR2L | BR | CHN1 | BL | CLIC2 | BR | COL1A1 | GY | CPT2 | BR | CTGF |
| BL | CDRT4 | GY | CHP2 | BL | CLIC5 | BR | COL1A2 | PI | CPVL | BR | CTHRC1 |
| MA | CDS1 | BR | CHPF2 | YE | CLIP2 | YE | COL22A1 | BR | CPXM1 | BL | CTLA4 |
| YE | CTNNAL1 | BR | CYB5R3 | BR | DCLK1 | MA | DGKA | BL | DNASE1L3 | BL | DUSP14 |
| BL | CTNS | BL | CYBASC3 | BR | DCLRE1C | BR | DGKD | GN | DNASE2 | MA | DUSP22 |
| YE | CTPS | PI | CYBB | BR | DCN | MA | DHCR24 | GN | DNM2 | BL | DUSP2 |
| PI | CTSB | BL | CYFIP2 | BL | DCP2 | BL | DHCR7 | BL | DOCK10 | BL | DUSP4 |
| PI | CTSC | YE | CYGB | GN | DCTD | GY | DHDDS | BL | DOCK11 | GY | DUSP5 |
| PI | CTSD | BR | CYP26B1 | RE | DCTN1 | GN | DHPS | GN | DOCK1 | MA | DUSP7 |
| YE | CTSE | YE | CYP27A1 | GN | DCTN2 | GN | DHRS11 | BL | DOCK2 | BL | DUSP9 |
| PI | CTSH | GY | CYP27C1 | BR | DCTN6 | MA | DHRS9 | BL | DOCK6 | BR | DVL2 |
| BR | CTSK | MA | CYP2C18 | GN | DCTPP1 | BL | DHX32 | BL | DOCK8 | BL | DYNLT3 |
| PI | CTSL1 | BR | CYP2R1 | PI | DCUN1D4 | RE | DHX34 | GN | DOHH | YE | DYRK1B |
| GN | CTSO | BL | CYP2S1 | GY | DDIT4 | GN | DHX37 | PI | DOK1 | BR | DYSF |
| PI | CTSS | BR | CYP2U1 | BR | DDR2 | BR | DIO2 | BL | DOK2 | BL | DZIP1L |
| BL | CTSW | GY | CYP4F11 | GY | DDX10 | GY | DIS3L2 | BL | DOK3 | YE | DZIP1 |
| PI | CTSZ | GY | CYP4F3 | RE | DDX11 | BR | DIXDC1 | BL | DOK4 | BR | E2F2 |
| BL | CTTN | BL | CYP4V2 | RE | DDX12 | BL | DKFZP586 [symbol] | [symbol] | DONSON | GN | EBAG9 |
| GN | CTU1 | YE | CYP4X1 | BL | DDX1 | GY | DKK1 | GN | DOT1L | BR | EBF1 |
| GN | CTXN1 | BL | CYP51A1 | GY | DDX23 | BR | DKK3 | GN | DPH1 | YE | EBF3 |
| BL | CUEDC1 | BR | CYR61 | GN | DDX39 | BR | DLC1 | GN | DPH2 | BL | EBI3 |
| BR | CUL7 | BL | CYTH4 | GY | DDX3Y | GY | DLD | GN | DPP3 | YE | ECE1 |
| GY | CUL9 | BL | CYTIP | GY | DDX47 | BR | DLEU2 | BR | DPP4 | RE | ECHDC2 |
| BR | CUX1 | BL | CYTSB | GN | DDX49 | BR | DLG3 | GN | DPP9 | BL | ECHDC3 |
| BR | CWC25 | BR | CYYR1 | GN | DDX54 | BR | DLG4 | BR | DPT | BR | ECM2 |
| BR | CWC27 | GY | CYorf15B | RE | DDX55 | BR | DLGAP4 | GN | DPY19L1 | GN | ECSIT |
| BL | CX3CL1 | RE | D2HGDH | GN | DDX59 | YE | DLK2 | YE | DPYSL2 | YE | EDARADD |
| BL | CX3CR1 | GY | D4S234E | BL | DEDD | YE | DLL1 | BR | DPYSL3 | RE | EDIL3 |
| BR | CXCL12 | BR | DAAM2 | BL | DEF6 | BR | DLL4 | MA | DQX1 | GY | EDN1 |
| BL | CXCL13 | GY | DAB2IP | BL | DEGS1 | GY | DLX5 | YE | DRAM1 | GY | EDN2 |
| MA | CXCL17 | BR | DAB2 | GY | DEGS2 | BL | DLX6 | GN | DSC2 | BR | EDNRA |
| BL | CXCL1 | BR | DACT1 | BR | DEM1 | YE | DMD | BR | DSCR6 | BR | EDNRB |
| YE | CXCL2 | YE | DACT2 | BL | DENND1C | GN | DMRTA1 | BR | DSEL | BR | EEPD1 |
| GY | CXCL6 | BR | DAPK1 | BL | DENND2D | BR | DMRTA2 | RE | DSE | BR | EFEMP1 |
| BL | CXCL9 | GN | DAPK3 | BL | DENND3 | PI | DMXL2 | MA | DSG3 | BR | EFEMP2 |
| BL | CXCR2P1 | MA | DAPP1 | BL | DENND4B | YE | DNAH11 | BL | DSTN | RE | EFHC1 |
| BL | CXCR3 | BR | DAP | BR | DENND5B | GY | DNAH17 | BL | DTX1 | YE | EFHD1 |
| BL | CXCR4 | BL | DARC | YE | DENR | BL | DNAH1 | BR | DTX2 | GY | EFHD2 |
| BL | CXCR5 | GN | DAZAP1 | YE | DEPDC7 | GY | DNAH5 | RE | DTX3 | BL | EFNA1 |
| BL | CXCR6 | BR | DBF4 | BL | DERA | GY | DNAJA3 | GY | DTX4 | MA | EFNA3 |
| RE | CXXC1 | BL | DBN1 | BL | DERL3 | BR | DNAJB5 | MA | DUOX1 | BL | EFNA5 |
| BR | CXXC5 | GY | DCAF11 | GY | DET1 | GY | DNAJB6 | MA | DUOX2 | GY | EFNB1 |
| BR | CXorf36 | GN | DCAF15 | GY | DFFA | BR | DNAJB9 | MA | DUOXA1 | GN | EFNB2 |
| GN | CXorf57 | BR | DCAF8 | BL | DFNA5 | YE | DNAJC18 | MA | DUOXA2 | BR | EFS |
| GY | CYB561D1 | GY | DCAKD | YE | DFNB31 | GY | DNAJC21 | GN | DUS3L | GY | EFTUD2 |
| GY | CYB5A | BR | DCHS1 | YE | DGAT2 | GN | DNAJC24 | GY | DUS4L | RE | EGFL8 |
| BL | CYB5R2 | GN | DCI | BL | DGCR2 | RE | DNAJC25 | GN | DUSP10 | YE | EGFLAM |
| RE | EHD2 | GN | ENPP5 | BL | EVI2B | GN | FAM149B1 | RE | FAM73B | GN | FBXW7 |
| BR | EHD3 | YE | ENPP6 | YE | EVI5L | RE | FAM156A | GY | FAM76A | GN | FBXW9 |
| BR | EID1 | BL | ENTPD1 | BL | EVL | GY | FAM160A2 | BL | FAM78A | BL | FCER1A |
| GY | EIF1AY | BL | EOMES | MA | EVPL | BL | FAM160B1 | BR | FAM81A | PI | FCER1G |
| GY | EIF2AK1 | MA | EPB41L1 | GN | EXOC1 | YE | FAM161A | MA | FAM83A | YE | FCGBP |
| GN | EIF3CL | PI | EPB41L3 | BL | EXOC6 | YE | FAM164A | MA | FAM83C | PI | FCGR1A |
| GN | EIF3G | GY | EPB41L4B | GY | EXOC7 | YE | FAM167A | BR | FAM83D | PI | FCGR1B |
| BL | EIF4E3 | BL | EPB49 | BR | EXOSC10 | YE | FAM171A1 | BR | FAM83E | PI | FCGR2A |
| GN | EIF4EBP1 | BL | EPCAM | GN | EXOSC3 | BR | FAM171B | BL | FAM83H | PI | FCGR2B |
| GY | EIF4E | BR | EPDR1 | RE | EXT1 | GY | FAM174A | GY | FAM86C | PI | FCGR3A |
| GN | ELAVL1 | MA | EPHA1 | BR | EXT2 | BR | FAM174B | BL | FAM89A | BL | FCHO1 |
| MA | ELF3 | MA | EPHA2 | RE | EXTL3 | BR | FAM176A | MA | FAM92A1 | RE | FCHSD1 |
| GN | ELL | BR | EPHA3 | BR | EYA2 | GN | FAM188A | RE | FAM98A | BL | FCRL5 |
| BL | ELMO1 | YE | EPHB1 | BR | EZH2 | GN | FAM188B | BR | FANCA | BL | FCRLA |
| RE | ELMOD3 | YE | EPHB2 | BL | F11R | YE | FAM189A2 | BR | FANCD2 | YE | FDFT1 |
| BR | ELN | MA | EPHB3 | BR | F13A1 | BL | FAM189B | BR | FANCE | GY | FECH |
| GN | ELOF1 | GY | EPHB4 | BR | F2RL2 | YE | FAM18B2 | PI | FANCF | RE | FER1L4 |
| BL | ELOVL4 | GY | EPHB6 | BR | F2R | RE | FAM193B | GN | FANCG | BR | FERMT2 |
| BL | ELOVL6 | BR | EPHX3 | BL | F3 | GN | FAM195A | RE | FANCL | BL | FERMT3 |
| YE | ELP2 | MA | EPN2 | BL | F5 | BR | FAM198B | BR | FAP | PI | FES |
| GN | ELP3 | MA | EPN3 | GY | FA2H | GN | FAM200A | GN | FARSA | GY | FEZ1 |
| BR | ELP4 | MA | EPS8L1 | YE | FAAH2 | BR | FAM20A | YE | FASN | GY | FEZ2 |
| BR | ELTD1 | MA | EPS8L2 | MA | FABP5 | BL | FAM21B | BR | FASTKD1 | BL | FGD2 |
| BL | EMB | BL | ERAP2 | GN | FADD | PI | FAM26F | RE | FASTKD3 | BL | FGD3 |
| BR | EMCN | PI | ERBB2 | MA | FADS1 | GN | FAM32A | YE | FAS | BR | FGD5 |
| RE | EME1 | BR | ERCC3 | GY | FADS2 | GY | FAM35A | BR | FBF1 | BL | FGF11 |
| BR | EMILIN1 | BL | ERCC5 | BL | FAIM3 | GY | FAM35B | BR | FBLN2 | YE | FGF1 |
| PI | EMILIN2 | BR | ERF | BR | FAIM | BL | FAM3C | BR | FBLN5 | YE | FGF2 |
| BR | EML1 | RE | ERGIC1 | BR | FAM101B | YE | FAM45A | GY | FBLN7 | BL | FGFBP1 |
| MA | EMP1 | GN | ERICH1 | GY | FAM104A | GN | FAM46A | BR | FBN1 | BR | FGFR1 |
| MA | EMP2 | BR | ERLEC1 | BL | FAM105A | BL | FAM46C | PI | FBP1 | BR | FGFR3 |
| PI | EMP3 | YE | ERO1LB | YE | FAM105B | GN | FAM48A | GN | FBRSL1 | BR | FGFRL1 |
| PI | EMR2 | GN | ERRFI1 | BL | FAM107A | BL | FAM49A | GY | FBRS | GY | FGGY |
| YE | EN2 | BR | ESAM | BL | FAM107B | GY | FAM49B | GN | FBXL12 | PI | FGL2 |
| BR | ENC1 | YE | ESR1 | GN | FAM108A1 | GN | FAM50B | GY | FBXL18 | PI | FGR |
| RE | ENDOD1 | BL | ESRP1 | BR | FAM108C1 | BL | FAM53B | GY | FBXL19 | GN | FHDC1 |
| GN | ENDOG | RE | ESYT1 | BR | FAM110B | BR | FAM54A | BR | FBXL7 | YE | FHOD3 |
| RE | ENGASE | GY | ETF1 | BR | FAM111A | BR | FAM55C | GY | FBXO18 | GY | FIG4 |
| BR | ENG | GN | ETFA | BL | FAM113B | BR | FAM57A | GN | FBXO25 | BR | FILIP1L |
| BR | ENOPH1 | BL | ETS1 | YE | FAM117A | GY | FAM65A | YE | FBXO2 | BR | FIP1L1 |
| RE | ENOSF1 | YE | ETV1 | YE | FAM117B | BL | FAM65B | GY | FBXO41 | GN | FIZ1 |
| BR | ENPEP | BR | ETV5 | GN | FAM125A | YE | FAM65C | MA | FBXO42 | YE | FJX1 |
| BR | ENPP1 | YE | ETV6 | BL | FAM125B | YE | FAM70A | GN | FBXO46 | BR | FKBP10 |
| BL | ENPP2 | BL | EVI2A | BL | FAM129B | BR | FAM72B | GN | FBXO8 | BL | FKBP11 |
| GY | FKBP4 | PI | FPR3 | YE | GALNTL4 | BL | GIMAP8 | RE | GNB1 | BR | GPR161 |
| BL | FKBP5 | YE | FRMD4A | BR | GAS1 | BR | GIN1 | BR | GNB5 | BL | GPR171 |
| BR | FKBP7 | BL | FRMD8 | BR | GAS7 | GN | GINS2 | BR | GNG11 | RE | GPR172B |
| GN | FKBP8 | BR | FRY | BR | GATA2 | YE | GINS3 | BL | GNG2 | RE | GPR176 |
| BR | FKBP9 | BL | FRZB | BL | GATA3 | MA | GIPC1 | BL | GNG7 | BL | GPR183 |
| GY | FKRP | BL | FSCN1 | YE | GATA6 | YE | GIPC2 | GN | GNPDA1 | BL | GPR34 |
| BL | FLI1 | BR | FSTL1 | GY | GATM | GN | GIT1 | BL | GNPNAT1 | YE | GPR39 |
| GY | FLII | BR | FSTL3 | RE | GBA2 | BL | GIT2 | RE | GNRHR2 | BR | GPR4 |
| BR | FLJ10357 | YE | FSTL4 | GY | GBAS | RE | GJA1 | RE | GNS | BL | GPR56 |
| GY | FLJ33630 | BR | FST | GY | GBA | YE | GJA3 | RE | GOLGA2B | BL | GPR65 |
| BL | FLJ40330 | PI | FTL | PI | GBGT1 | BR | GJA4 | MA | GOLGA6L10 | GY | GPR68 |
| RE | FLJ45445 | BL | FTSJ1 | BL | GBP3 | BR | GJA5 | MA | GOLGA6L9 | BL | GPR87 |
| BL | FLJ90757 | GN | FTSJ3 | BL | GBP4 | BR | GJB3 | BR | GOLGA7B | GN | GPR98 |
| BR | FLRT2 | GN | FUBP1 | BL | GCA | YE | GJB4 | RE | GOLGA8A | BL | GPRC5A |
| RE | FLRT3 | BL | FUCA1 | GN | GCDH | MA | GJB5 | RE | GOLGA8B | BR | GPRC5B |
| BR | FLT1 | YE | FUCA2 | YE | GCET2 | BR | GJC1 | BL | GOLM1 | GY | GPRC5C |
| BR | FLT4 | MA | FUT2 | BR | GCH1 | BL | GJD3 | YE | GORAB | YE | GPRIN2 |
| PI | FLVCR2 | MA | FUT3 | BL | GCNT1 | BR | GK | BR | GOSR2 | BL | GPSM3 |
| BL | FMNL1 | YE | FUT4 | GY | GCNT3 | GY | GLB1L | GN | GOT1 | BR | GPX3 |
| BL | FMNL3 | MA | FUT6 | PI | GDA | BL | GLCCI1 | BL | GPAT2 | GY | GPX7 |
| BR | FMO1 | GY | FXC1 | GY | GDE1 | BR | GLE1 | BR | GPATCH1 | BR | GPX8 |
| BR | FMO2 | GN | FXN | GN | GDF11 | BR | GLI2 | BL | GPC1 | YE | GRAMD1A |
| BR | FMOD | BL | FXYD5 | BL | GDF15 | GN | GLI3 | RE | GPC2 | YE | GRAMD3 |
| BR | FN1 | YE | FXYD6 | BR | GEFT | PI | GLIPR1 | BR | GPC4 | YE | GRAMD4 |
| GY | FN3KRP | BL | FYB | BR | GEN1 | PI | GLIPR2 | BR | GPC6 | BL | GRAP2 |
| GN | FN3K | BL | FYN | BL | GFI1 | BR | GLIS2 | MA | GPCPD1 | BL | GRAP |
| BL | FNBP1 | BR | FZD10 | MA | GFOD2 | BR | GLIS3 | GY | GPER | BL | GRB2 |
| BR | FNDC1 | BR | FZD4 | YE | GFPT2 | BR | GLOD4 | GN | GPI | BR | GRB7 |
| GY | FOLH1 | YE | FZD7 | BR | GFRA1 | BL | GLRX | GN | GPN3 | BR | GREM1 |
| PI | FOLR2 | YE | FZD8 | YE | GGA2 | RE | GLS2 | YE | GPNMB | MA | GRHL1 |
| BL | FOSL1 | GN | FZR1 | RE | GGCX | GN | GLS | GN | GPR108 | MA | GRHL3 |
| GY | FOXA1 | BR | G0S2 | GY | GGPS1 | GY | GLT25D1 | BR | GPR109A | YE | GRIN2A |
| GN | FOXC1 | BL | GAB3 | YE | GGT1 | BR | GLT8D2 | BR | GPR109B | GN | GRIN2C |
| BL | FOXD1 | BR | GABARAPL1 | BR | GGT5 | GN | GLTSCR1 | MA | GPR110 | BL | GRIN2D |
| BR | FOXF1 | RE | GABBR1 | MA | GGT6 | GY | GLUD1 | BL | GPR114 | BR | GRK5 |
| BR | FOXF2 | GY | GABRP | PI | GGTA1 | PI | GM2A | MA | GPR115 | BR | GRPEL2 |
| GY | FOXJ1 | MA | GABRQ | GN | GHITM | BR | GMEB2 | BR | GPR116 | RE | GRSF1 |
| GN | FOXK2 | GN | GADD45GIP1 | RE | GIGYF1 | BL | GMFG | BR | GPR124 | BR | GRTP1 |
| YE | FOXN1 | GN | GALC | BL | GIMAP1 | BL | GMIP | BL | GPR132 | GN | GRWD1 |
| BR | FOXP1 | MA | GALE | BL | GIMAP2 | PI | GMPR | BR | GPR137B | RE | GSDMB |
| BL | FOXP3 | BL | GALM | BL | GIMAP4 | GN | GNA12 | BR | GPR137C | MA | GSDMC |
| YE | FOXP4 | YE | GALNT11 | BL | GIMAP5 | MA | GNA15 | BL | GPR153 | GN | GSK3A |
| GY | FOXQ1 | RE | GALNT2 | BL | GIMAP6 | PI | GNAI2 | BL | GPR155 | BR | GSPT2 |
| PI | FPR1 | BR | GALNT6 | BL | GIMAP7 | BR | GNAO1 | YE | GPR160 | GN | GSS |
| BL | GSTM1 | GN | HDGFRP2 | BR | HMCN1 | GN | HSPA2 | BL | IGJ | BR | INHBB |
| GY | GSTT1 | GY | HDHD1A | GN | HMG20B | RE | HSPA4 | PI | IGSF6 | BR | INMT |
| BR | GTDC1 | BL | HDHD2 | BL | HMGA2 | GN | HSPA6 | GY | IKBIP | YE | INPP1 |
| GN | GTF2F1 | BR | HDLBP | BR | HMGB2 | RE | HSPA8 | BL | IKBKB | YE | INPP4A |
| GY | GTF2H2B | GY | HECTD3 | RE | HMGN1 | BR | HSPA9 | YE | IKBKE | BL | INPP5A |
| BL | GTF2IRD1 | RE | HEG1 | PI | HMGN5 | YE | HSPB8 | BL | IKZF1 | BL | INPP5B |
| PI | GTF2IRD2B | GN | HELQ | BL | HMHA1 | GN | HSPBP1 | BL | IL10RA | BL | INPP5D |
| RE | GTF2IRD2P1 | BR | HEPH | PI | HMOX1 | GN | HSPD1 | RE | IL11RA | RE | INPP5E |
| RE | GTPBP3 | GN | HERC4 | PI | HN1L | GY | HSPH1 | BL | IL12RB1 | GY | INPP5F |
| BL | GTPBP4 | BL | HERPUD1 | PI | HNMT | BR | HTRA1 | RE | IL13RA1 | GN | INSIG2 |
| BR | GUCY1A3 | BL | HES1 | BR | HNRNPA2B1 | BR | HTRA3 | YE | IL15 | YE | INTS12 |
| YE | GUCY1B3 | MA | HES2 | BR | HNRNPF | YE | HUNK | BL | IL16 | GN | INTS5 |
| RE | GUSBP1 | PI | HEXA | RE | HNRNPH1 | GN | HUS1 | RE | IL17RB | GN | INTS9 |
| BL | GVIN1 | RE | HEXDC | RE | HNRNPL | BL | HVCN1 | YE | IL17REL | GN | INTU |
| BR | GYG2 | YE | HEY1 | GN | HNRNPM | BL | HYAL1 | MA | IL17RE | BL | IPCEF1 |
| YE | GYLTL1B | GY | HEY2 | RE | HNRPDL | BR | HYOU1 | BL | IL18BP | BR | IPO13 |
| BL | GYPC | BR | HEYL | MA | HOMER2 | YE | ICAM1 | YE | IL18R1 | GY | IPO4 |
| MA | GZF1 | GN | HGS | BR | HOMEZ | BL | ICAM2 | PI | IL18 | MA | IPPK |
| BL | GZMA | BL | HHEX | RE | HOOK2 | BL | ICAM3 | GN | IL1A | RE | IQCC |
| BL | GZMB | GN | HIBCH | GY | HOXA10 | YE | ICAM5 | GY | IL1B | YE | IQCE |
| BL | GZMH | BR | HIC1 | GY | HOXA3 | GY | ICMT | BR | IL1R1 | BR | IQCG |
| BL | GZMK | GN | HIST1H1C | YE | HOXB13 | YE | ICOSLG | YE | IL1R2 | PI | IQGAP2 |
| BL | GZMM | GY | HIST1H2AC | BR | HOXB2 | BL | ICOS | BR | IL20RA | YE | IRAK2 |
| BL | H19 | GY | HIST1H2BJ | YE | HOXC6 | BL | ID2 | MA | IL20RB | BL | IRAK4 |
| GY | H2AFV | YE | HIVEP3 | GY | HOXD10 | GY | IDH3A | BL | IL21R | BL | IRF1 |
| YE | H2AFY2 | RE | HK1 | GY | HOXD11 | MA | IDI1 | YE | IL23A | GN | IRF2 |
| BL | HAAO | PI | HK3 | GY | HOXD13 | GN | IDS | YE | IL27RA | BL | IRF4 |
| BL | HADHB | BL | HKR1 | BR | HPGD | GN | IER2 | BL | IL2RA | PI | IRF5 |
| BR | HADH | BL | HLA.DMA | GN | HPS4 | BL | IER3 | BL | IL2RB | BL | IRF8 |
| PI | HAPLN3 | PI | HLA.DMB | BR | HR | PI | IFFO1 | BL | IL2RG | YE | IRS2 |
| BR | HAT1 | BL | HLA.DOA | MA | HS3ST1 | PI | IFI30 | PI | IL32 | BR | IRX4 |
| BR | HAUS3 | BL | HLA.DOB | PI | HS3ST3A1 | PI | IFITM2 | BL | IL3RA | YE | IRX5 |
| RE | HAUS5 | PI | HLA.DPA1 | BL | HS3ST4 | BL | IFNAR2 | PI | IL4I1 | YE | ISL1 |
| GN | HAUS8 | PI | HLA.DPB1 | PI | HS6ST1 | GN | IFNGR1 | GY | IL4R | BR | ISLR |
| PI | HAVCR2 | PI | HLA.DQA1 | YE | HS6ST2 | BR | IFRD1 | YE | IL7 | BR | ISOC1 |
| GY | HBA2 | BL | HLA.DQA2 | BR | HSD17B11 | GY | IFT74 | BL | IL8 | BR | ITGA11 |
| GY | HBB | PI | HLA.DQB1 | BL | HSD17B12 | YE | IFT88 | BR | ILDR1 | BR | ITGA1 |
| PI | HCG11 | BL | HLA.DQB2 | PI | HSD17B14 | BR | IGF2 | RE | ILF3 | BL | ITGA4 |
| BL | HCK | PI | HLA.DRA | RE | HSF4 | BL | IGFBP2 | GN | ILVBL | BR | ITGA5 |
| BL | HCLS1 | PI | HLA.DRB1 | BL | HSH2D | BR | IGFBP3 | GN | IMMT | BL | ITGAE |
| RE | HCN3 | PI | HLA.DRB5 | BR | HSPA12B | BR | IGFBP4 | GY | INA | BL | ITGAL |
| BL | HCST | BL | HLA.DRB6 | BR | HSPA14 | BR | IGFBP5 | YE | ING1 | YE | ITGAM |
| MA | HDAC1 | YE | HLF | GY | HSPA1A | BR | IGFBP7 | RE | ING5 | GN | ITGAV |
| GY | HDAC2 | BL | HLX | GN | HSPA1B | BL | IGHMBP2 | BR | INHBA | PI | ITGAX |
| RE | ITGB1 | RE | KCNJ15 | BL | KIAA1274 | BR | KRT15 | GN | LENG9 | BL | LMO2 |
| PI | ITGB2 | YE | KCNJ5 | BL | KIAA1279 | BL | KRT17 | YE | LEO1 | BR | LMO4 |
| RE | ITGB3BP | BR | KCNJ8 | GY | KIAA1324 | GN | KRT18 | BR | LEPRE1 | BR | LMOD1 |
| BR | ITGB3 | BL | KCNK1 | BR | KIAA1462 | BR | KRT19 | BR | LEPREL2 | BL | LMTK3 |
| GY | ITGB4 | BL | KCNK5 | RE | KIAA1529 | BR | KRT24 | BL | LEPROTL1 | BL | LNP1 |
| BL | ITGB5 | MA | KCNK6 | YE | KIAA1543 | GY | KRT31 | GN | LEPR | PI | LNX1 |
| GN | ITGB6 | YE | KCNMA1 | MA | KIAA1609 | BR | KRT5 | BL | LETM1 | BL | LOC100125556 |
| BL | ITGB7 | BR | KCNN3 | BR | KIAA1644 | GY | KRT7 | RE | LETMD1 | BR | LOC100128191 |
| BR | ITGBL1 | BL | KCNN4 | RE | KIAA1683 | GN | KRT8 | BL | LFNG | PI | LOC100129034 |
| BR | ITIH5 | GY | KCNQ1 | | | YE | KSR1 | BL | LGALS2 | RE | LOC100129637 |
| BL | ITK | BL | KCNS1 | | | GY | KYNU | BL | LGALS9 | GN | LOC100130776 |
| BL | ITM2A | GY | KCNS3 | GN | KIAA1712 | YE | L3MBTL4 | YE | LGI2 | RE | LOC100132287 |
| GY | ITM2B | RE | KCTD10 | PI | KIAA1841 | PI | LACTB | MA | LGI3 | RE | LOC100133161 |
| BL | ITM2C | BL | KCTD11 | BL | KIAA1949 | MA | LAD1 | PI | LGMN | RE | LOC100133331 |
| MA | ITPKC | PI | KCTD12 | GN | KIAA1967 | BL | LAG3 | BR | LGR5 | GY | LOC100134229 |
| BL | ITPR1 | RE | KCTD13 | GY | KIAA2022 | PI | LAIR1 | PI | LHFPL2 | MA | LOC100190939 |
| GN | ITPRIPL1 | YE | KCTD15 | YE | KIF21A | BR | LAMA1 | GY | LHFPL4 | RE | LOC100216545 |
| BR | IVNS1ABP | BL | KDELC1 | BL | KIF21B | BR | LAMA2 | BR | LHFP | GN | LOC113230 |
| BL | IWS1 | BL | KDELR2 | BR | KIF26A | BR | LAMA4 | BL | LHX6 | RE | LOC115110 |
| YE | JAG2 | BR | KDELR3 | BR | KIF26B | BR | LAMB1 | YE | LIFR | RE | LOC146880 |
| BL | JAK2 | BL | KDM1A | BR | KIF2C | BR | LAMB2 | BR | LIF | RE | LOC150776 |
| BL | JAK3 | GY | KDM5D | BR | KIF3C | GY | LAMB3 | RE | LIG1 | BR | LOC151162 |
| BR | JAM3 | BR | KDR | YE | KIFAP3 | GY | LAMP1 | PI | LILRB1 | RE | LOC162632 |
| YE | JAZF1 | GY | KDSR | BR | KIFC1 | BL | LAPTM4B | PI | LILRB2 | RE | LOC220594 |
| BL | JMJD5 | GN | KEAP1 | RE | KIFC2 | PI | LAPTM5 | PI | LILRB3 | BR | LOC254559 |
| RE | JMJD7.PLA [symbol] | [symbol] | KEL | BR | KIN | YE | LARGE | PI | LILRB4 | YE | LOC283070 |
| BL | JSRP1 | GY | KHDRBS1 | BL | KLC2 | YE | LARP6 | BR | LIMCH1 | YE | LOC283174 |
| GY | JUB | GY | KHDRBS3 | BL | KLC3 | BR | LARP7 | BL | LIMD2 | YE | LOC283267 |
| GN | JUNB | GN | KIAA0020 | GN | KLF16 | MA | LASS3 | BL | LIME1 | RE | LOC285074 |
| GN | JUND | BL | KIAA0040 | BL | KLF2 | BL | LAT2 | RE | LIMK1 | RE | LOC338799 |
| MA | JUP | GN | KIAA0114 | BL | KLF4 | BL | LAT | BL | LIMK2 | RE | LOC339047 |
| BR | KAL1 | BL | KIAA0125 | YE | KLHDC7B | BL | LAX1 | BR | LIMS2 | RE | LOC349114 |
| BR | KALRN | GY | KIAA0141 | RE | KLHL17 | BR | LAYN | BR | LINS1 | BL | LOC374443 |
| BR | KATNA1 | BR | KIAA0195 | YE | KLHL29 | BL | LBH | PI | LIPA | BR | LOC387647 |
| MA | KAZ | GN | KIAA0319L | GN | KLHL2 | BL | LCK | GN | LIPE | MA | LOC388152 |
| GN | KBTBD2 | GY | KIAA0391 | BL | KLHL6 | BL | LCLAT1 | GN | LIPG | BL | LOC388692 |
| BL | KBTBD8 | BR | KIAA0427 | BL | KLRB1 | MA | LCN2 | MA | LIPH | BL | LOC399744 |
| BL | KCNAB2 | YE | KIAA0649 | GY | KLRG2 | BL | LCP1 | RE | LIPT1 | YE | LOC399959 |
| RE | KCNC3 | GN | KIAA0664 | BL | KLRK1 | BL | LCP2 | YE | LITAF | RE | LOC400027 |
| YE | KCNC4 | BL | KIAA0748 | GN | KRCC1 | BR | LDB2 | BL | LIX1L | BL | LOC400657 |
| YE | KCND1 | RE | KIAA0895L | YE | KREMEN2 | GY | LDHA | BL | LLGL2 | YE | LOC401093 |
| BR | KCNE4 | BL | KIAA0895 | GN | KRI1 | BL | LEF1 | BR | LMCD1 | BL | LOC401397 |
| YE | KCNIP3 | RE | KIAA0907 | BL | KRT10 | YE | LEMD1 | BL | LMNA | GN | LOC407835 |
| GY | KCNJ11 | BL | KIAA0922 | MA | KRT13 | RE | LENG8 | GN | LMNB2 | GY | LOC440173 |
| RE | LOC440944 | BR | LRRC15 | PI | LYZ | GN | MAPKAPK5 | BR | MEIS1 | GY | MKNK1 |
| GN | LOC550112 | BL | LRRC1 | YE | LZTS1 | GN | MAPKBP1 | GN | MEIS2 | PI | MKS1 |
| GY | LOC595101 | PI | LRRC25 | MA | MACC1 | GN | MAPKSP1 | BR | MELK | BR | MLF1IP |
| BL | LOC606724 | RE | LRRC28 | BR | MAD2L1 | BR | MAPRE3 | BR | MEN1 | BR | MLF1 |
| RE | LOC642846 | BR | LRRC32 | MA | MADD | PI | MARCO | BL | MEOX1 | BR | MLLT11 |
| GY | LOC654433 | BL | LRRC33 | BL | MAFF | BR | MARK1 | YE | MERTK | GN | MLLT1 |
| YE | LOC728392 | BR | LRRC37B2 | BR | MAFG | BR | MARK4 | GY | MESDC2 | MA | MLLT3 |
| BR | LOC728554 | BL | LRRC42 | BL | MAFK | GN | MARS | RE | METT11D1 | BL | MLLT6 |
| GY | LOC728613 | YE | LRRC49 | YE | MAF | BR | MARVELD1 | RE | METTL10 | GY | MLPH |
| GN | LOC72991 [symbol] | [symbol] | LRRC4 | BL | MAGED1 | BL | MAST3 | YE | METTL13 | GN | MMAA |
| GY | LOC730101 | BL | LRRC59 | BR | MAGED4B | PI | MASTL | BL | METTL2A | BL | MMADHC |
| MA | LOC80154 | BL | LRRC8A | GN | MAGED4 | BR | MAT2A | RE | METTL3 | YE | MMD |
| BR | LOC81691 | BL | LRRC8E | GY | MAGEE1 | YE | MAT2B | BL | METTL7A | BR | MME |
| YE | LOC84740 | YE | LSAMP | BR | MAGEH1 | BL | MATK | GY | METTL9 | BL | MMP10 |
| YE | LOC84856 | GN | LSM4 | GY | MAL2 | YE | MATN2 | BL | MEX3D | BR | MMP11 |
| GY | LOC90784 | GN | LSM7 | RE | MALAT1 | BR | MAVS | BR | MFAP2 | PI | MMP12 |
| RE | LOC91316 | BL | LSP1 | MA | MALL | BL | MAX | BR | MFAP4 | BR | MMP13 |
| BL | LOC96610 | GN | LSR | BL | MALT1 | GN | MAZ | BR | MFAP5 | BR | MMP14 |
| GN | LONP1 | PI | LST1 | RE | MAMDC4 | BR | MBD1 | BR | MFGE8 | BL | MMP15 |
| BR | LOXL1 | GY | LTB4R2 | YE | MAMLD1 | GN | MBD3 | BL | MFNG | YE | MMP19 |
| BR | LOXL2 | BR | LTBP2 | BL | MAN1C1 | YE | MBNL2 | BR | MFRP | BR | MMP1 |
| BR | LOXL3 | BR | LTBP3 | BL | MAN2A2 | GY | MBOAT1 | YE | MFSD2A | PI | MMP25 |
| BR | LOXL4 | GY | LTBP4 | BL | MAN2B1 | MA | MBOAT2 | PI | MFSD7 | YE | MMP28 |
| MA | LPAR5 | BL | LTBR | PI | MANBA | BR | MCAM | GN | MFSD8 | BR | MMP2 |
| PI | LPCAT1 | BL | LTB | GY | MANEAL | YE | MCF2L | YE | MGAT3 | BR | MMP3 |
| YE | LPCAT4 | YE | LTF | GN | MAOB | BR | MCM3 | BL | MGAT4A | PI | MMP9 |
| GN | LPHN1 | GY | LTV1 | BR | MAP1A | BR | MCM5 | YE | MGC2752 | BR | MMRN2 |
| GN | LPHN2 | RE | LUC7L3 | BR | MAP1B | GN | MCM7 | BL | MGC29506 | BR | MN1 |
| BL | LPIN1 | RE | LUC7L | GN | MAP1S | GN | MCOLN1 | PI | MGC57346 | PI | MNDA |
| YE | LPIN2 | BR | LUM | GN | MAP2K2 | BL | MCOLN2 | BR | MGP | BR | MNS1 |
| RE | LPIN3 | GY | LXN | GN | MAP2K5 | BR | ME3 | BL | MIAT | BL | MOBKL2A |
| BR | LPL | BL | LY86 | GN | MAP2K7 | GY | MEAF6 | BL | MICAL1 | YE | MOBKL2B |
| BL | LPPR2 | PI | LY96 | BR | MAP2 | GN | MED16 | BR | MICAL2 | BR | MOBKL2C |
| BL | LPXN | BL | LY9 | MA | MAP3K12 | PI | MED24 | GN | MICAL3 | GN | MOBKL3 |
| GY | LRAT | BL | LYL1 | YE | MAP3K14 | GN | MED25 | MA | MICALL1 | BR | MOCS1 |
| RE | LRDD | BR | LYPD1 | BL | MAP4K1 | RE | MED26 | YE | MICALL2 | BL | MORC2 |
| GN | LRFN3 | MA | LYPD3 | YE | MAP7D2 | GY | MED29 | GY | MID1IP1 | BL | MORF4L2 |
| MA | LRG1 | BL | LYPD6B | GN | MAP7D3 | GN | MED30 | GN | MID1 | YE | MOXD1 |
| BR | LRIG1 | BL | LYPLA1 | GN | MAP9 | BR | MED6 | GN | MIER2 | PI | MPEG1 |
| BL | LRMP | MA | LYPLA2P1 | MA | MAPK13 | BL | MEF2B | YE | MINA | GY | MPHOSPH10 |
| RE | LRP10 | GN | LYRM1 | GY | MAPK7 | YE | MEGF10 | GY | MINPP1 | YE | MPI |
| BL | LRP11 | GY | LYRM2 | GN | MAPK8IP2 | YE | MEGF6 | GN | MIOS | GN | MPND |
| RE | LRP1 | BR | LYRM5 | RE | MAPK8IP3 | YE | MEGF8 | RE | MITD1 | PI | MPP1 |
| GN | LRP3 | BL | LYSMD1 | GY | MAPK9 | RE | MEI1 | YE | MKL1 | RE | MPP3 |
| YE | MPP6 | GN | MT1G | RE | MZF1 | YE | NEDD1 | BR | NMNAT1 | YE | NTN1 |
| RE | MPPE1 | BR | MTA2 | BL | N4BP2L1 | BL | NEDD4L | YE | NMT2 | BR | NTN4 |
| GN | MPRIP | BL | MTA3 | BL | N4BP2L2 | BR | NEFH | BR | NNMT | YE | NTRK2 |
| GN | MPV17L2 | GN | MTERFD1 | BL | NAAA | GY | NEFL | PI | NOD1 | YE | NTS |
| BR | MPZL1 | BL | MTERFD2 | YE | NACC1 | GY | NEIL2 | RE | NOMO1 | YE | NUAK1 |
| MA | MPZL2 | RE | MTERFD3 | PI | NADK | BR | NEK11 | RE | NOMO3 | GN | NUAK2 |
| GN | MR1 | YE | MTHFD1L | RE | NAPB | PI | NEK6 | GY | NOP14 | BL | NUB1 |
| BR | MRAS | BR | MTHFD2 | BL | NAPSB | BL | NEK8 | GY | NOP2 | GN | NUBP1 |
| PI | MRC1 | GY | MTIF2 | RE | NASP | GY | NELL2 | BR | NOS2 | GY | NUBPL |
| BR | MRC2 | PI | MTL5 | GY | NAT1 | RE | NEURL4 | BR | NOS3 | BR | NUDCD3 |
| BR | MRGPRF | GN | MTMR11 | YE | NAV2 | BR | NF2 | YE | NOTCH4 | MA | NUDT11 |
| RE | MRI1 | BL | MTSS1L | MA | NBEAL2 | PI | NFAM1 | YE | NOV | BL | NUDT12 |
| GN | MRPL10 | BR | MTX2 | GN | NBEA | GY | NFATC1 | BR | NOX4 | GY | NUDT15 |
| GN | MRPL11 | YE | MUC15 | YE | NCALD | BR | NFATC4 | YE | NOXO1 | GY | NUDT19 |
| GN | MRPL13 | MA | MUC20 | BR | NCAPG | YE | NFE2L3 | BR | NPAS2 | BR | NUF2 |
| GN | MRPL15 | GN | MUM1 | GY | NCDN | GN | NFIA | RE | NPIPL3 | GY | NUFIP1 |
| GN | MRPL34 | RE | MUS81 | BL | NCF1C | MA | NFIB | RE | NPIP | BL | NUMBL |
| BR | MRPL35 | MA | MXD1 | BL | NCF1 | YE | NFIL3 | BL | NPLOC4 | BR | NUP210 |
| BR | MRPL39 | BL | MXD4 | PI | NCF2 | GN | NFKB1 | PI | NPL | BR | NUP35 |
| BL | MRPL44 | BR | MXRA5 | BL | NCF4 | YE | NFKB2 | BR | NPM2 | YE | NUP50 |
| BL | MRPL49 | BR | MXRA7 | BL | NCKAP1L | YE | NFKBIA | YE | NPNT | GN | NUP54 |
| GN | MRPL4 | BR | MXRA8 | BR | NCKAP5L | BL | NFKBID | BR | NPR1 | RE | NUPL2 |
| BR | MRPL50 | BR | MYADM | GN | NCKAP5 | YE | NFKBIE | YE | NPTXR | BR | NUSAP1 |
| GN | MRPL54 | GN | MYBBP1A | GN | NCLN | BR | NFS1 | BL | NR1D1 | RE | NVL |
| GN | MRPS12 | YE | MYBL1 | GN | NCRNA00174 | RE | NFYB | GN | NR1H2 | RE | NXF1 |
| GN | MRPS30 | GY | MYB | RE | NCRNA00201 | BL | NGEF | PI | NR1H3 | YE | NXN |
| GN | MRPS35 | MA | MYCBP | BL | NCS1 | YE | NGFR | GN | NR2C2AP | GN | NXPH4 |
| BR | MRRF | GY | MYCL1 | BR | NDC80 | GY | NHEJ1 | BR | NR2F1 | PI | NYNRIN |
| BR | MRVI1 | YE | MYCN | GY | NDNL2 | BL | NHLRC3 | GN | NR2F6 | BR | OAF |
| BL | MS4A1 | GY | MYC | BR | NDN | BR | NID1 | BR | NR4A3 | GN | OAZ1 |
| PI | MS4A4A | PI | MYEOV | GY | NDRG1 | BR | NID2 | BL | NRARP | BR | OAZ2 |
| PI | MS4A6A | BR | MYH11 | MA | NDRG2 | BR | NIF3L1 | YE | NRCAM | GY | OBFC2A |
| PI | MS4A7 | BL | MYH14 | GY | NDRG4 | YE | NINJ1 | BL | NRIP3 | BR | OBSL1 |
| BR | MSC | RE | MYH9 | BL | NDST2 | PI | NINJ2 | BR | NRP1 | YE | OCA2 |
| RE | MSH5 | RE | MYL5 | GN | NDUFA11 | RE | NINL | RE | NSMCE4A | YE | ODC1 |
| GY | MSL3L2 | BR | MYL9 | GN | NDUFA13 | BL | NIPSNAP1 | BL | NSUN2 | MA | ODF2L |
| BL | MSL3 | BR | MYLK | GY | NDUFA4L2 | BL | NKG7 | RE | NSUN5P1 | GY | ODF2 |
| GN | MSLN | RE | MYO15B | GN | NDUFA7 | BL | NKIRAS2 | RE | NSUN5P2 | GN | ODZ2 |
| YE | MSMB | BR | MYO19 | GN | NDUFAB1 | GY | NKX3.1 | BR | NSUN6 | YE | ODZ3 |
| PI | MSR1 | BL | MYO1F | GN | NDUFB7 | GY | NLGN4Y | BR | NSUN7 | BR | ODZ4 |
| BR | MSRB3 | BL | MYO1G | GN | NDUFS7 | GN | NLK | YE | NT5DC1 | RE | OFD1 |
| RE | MST1P2 | GY | MYO3A | RE | NEAT1 | BL | NLRC3 | MA | NT5DC3 | RE | OGFOD2 |
| YE | MST1R | PI | MYO7A | GN | NECAB1 | YE | NLRP1 | BR | NT5E | BL | OGFRL1 |
| YE | MSTO1 | BL | MYO9B | BL | NECAP2 | PI | NLRP2 | BR | NTM | BR | OIP5 |
| YE | OLFM1 | MA | PAFAH2 | BR | PCDH17 | MA | PDZK1IP1 | GY | PIGG | RE | PLBD2 |
| BR | OLFM2 | BL | PAG1 | BR | PCDH18 | BR | PDZRN3 | GY | PIGR | BL | PLCB2 |
| BR | OLFML1 | BL | PAIP1 | GN | PCDH1 | BR | PEA15 | BL | PIK3AP1 | BL | PLCB3 |
| GN | OLFML2A | BL | PAIP2B | RE | PCDH7 | PI | PECAM1 | GN | PIK3C2B | BL | PLCD3 |
| BR | OLFML2B | GY | PAK1IP1 | BL | PCDHB14 | BR | PECR | BL | PIK3CD | BL | PLCG2 |
| BR | OLFML3 | YE | PAK1 | RE | PCDHGC3 | BR | PEG10 | BL | PIK3CG | BR | PLCH2 |
| PI | OLR1 | BL | PAK4 | BL | PCGF2 | GN | PELI2 | BL | PIK3IP1 | YE | PLCL1 |
| GY | OMA1 | YE | PAK6 | GY | PCGF3 | MA | PERP | GY | PIK3R2 | BL | PLCL2 |
| YE | ORAI2 | BR | PALM2.AK [symbol] | [symbol] | PCGF6 | BL | PEX11A | BL | PIK3R5 | RE | PLCXD1 |
| BL | ORAI3 | GN | PALMD | GY | PCID2 | BR | PEX5 | PI | PILRA | PI | PLD3 |
| PI | ORC4L | BR | PALM | GY | PCNT | BR | PEX6 | RE | PILRB | BL | PLD4 |
| GN | ORC5L | YE | PAMR1 | BL | PCNXL3 | GY | PEX7 | MA | PIM1 | YE | PLD6 |
| BR | ORC6L | RE | PANX1 | GY | PCOLCE2 | YE | PFKFB4 | BL | PIM2 | RE | PLEC |
| RE | ORMDL1 | YE | PAPLN | BR | PCOLCE | BL | PFKP | GN | PIN1 | BL | PLEKHA2 |
| BL | OSBP2 | GN | PAPPA | GY | PCP4L1 | GY | PFN2 | BL | PION | GN | PLEKHA3 |
| BR | OSBPL5 | YE | PAPSS1 | BR | PCSK5 | BR | PGAP2 | BL | PIP4K2A | BL | PLEKHA6 |
| RE | OSBPL7 | YE | PAPSS2 | BL | PCSK7 | GY | PGBD1 | BR | PIR | BL | PLEKHB1 |
| PI | OSCAR | BR | PAQR4 | GY | PCTP | BR | PGBD2 | YE | PISD | YE | PLEKHF1 |
| YE | OSTF1 | GY | PAQR5 | BR | PCYOX1L | GN | PGBD3 | BL | PITPNB | BR | PLEKHG3 |
| BR | OSTM1 | BL | PAQR7 | PI | PDCD1LG2 | BR | PGCP | BL | PITPNC1 | GY | PLEKHG4B |
| GN | OSTalpha | BL | PAQR8 | BL | PDCD1 | MA | PGD | GY | PITPNM1 | BR | PLEKHG4 |
| MA | OTUB2 | BL | PARD3 | GY | PDCD7 | GN | PGLS | RE | PITRM1 | GN | PLEKHG5 |
| BL | OTUD1 | GY | PARD6B | RE | PDDC1 | MA | PGLYRP3 | GN | PITX1 | PI | PLEKHG6 |
| MA | OVOL1 | GY | PARD6G | GN | PDE10A | BL | PGM1 | BR | PITX2 | GN | PLEKHH2 |
| GN | OVOL2 | YE | PARM1 | BR | PDE2A | RE | PGM2L1 | YE | PJA1 | GN | PLEKHJ1 |
| YE | OXCT1 | RE | PARP10 | BR | PDE4A | BL | PGPEP1 | GN | PKD2 | MA | PLEKHN1 |
| BL | P2RX5 | BL | PARP11 | BR | PDE4B | GN | PGRMC2 | YE | PKDCC | PI | PLEKHO1 |
| BL | P2RY10 | BR | PARP16 | GY | PDE7A | RE | PGS1 | BR | PKIG | BL | PLEKHO2 |
| GN | P2RY11 | BR | PARP2 | GY | PDE9A | GN | PHB | GY | PKNOX1 | BL | PLEK |
| BL | P2RY13 | RE | PARP6 | BR | PDGFA | YE | PHC1 | MA | PKP1 | PI | PLIN2 |
| MA | P2RY2 | BR | PARS2 | BR | PDGFB | YE | PHF10 | YE | PKP2 | MA | PLIN3 |
| PI | P2RY6 | GY | PART1 | BR | PDGFC | BL | PHF13 | BL | PKP3 | YE | PLK1 |
| BL | P2RY8 | BL | PARVG | BR | PDGFRA | GY | PHF15 | GN | PKP4 | GY | PLK2 |
| GY | P4HA1 | GY | PAX1 | BR | PDGFRB | BL | PHF17 | BL | PLA2G12A | GN | PLLP |
| BL | P4HA2 | BL | PAX5 | BR | PDGFRL | RE | PHKA2 | BL | PLA2G2D | BR | PLOD1 |
| GN | PA2G4P4 | GY | PAX6 | RE | PDIA4 | BL | PHLDA1 | BR | PLA2G3 | GN | PLRG1 |
| GN | PA2G4 | GY | PAX8 | BL | PDIA5 | BL | PHLDA2 | YE | PLA2G4C | PI | PLTP |
| RE | PABPC1L | BR | PAX9 | RE | PDIA6 | BR | PHLDB1 | MA | PLA2G4F | BR | PLVAP |
| GN | PABPC4L | BR | PBK | GY | PDK2 | GY | PHYHD1 | RE | PLA2G6 | BR | PLXDC1 |
| RE | PABPN1 | BR | PBXIP1 | GY | PDK3 | RE | PI4KAP1 | PI | PLA2G7 | BR | PLXDC2 |
| GY | PACS1 | BR | PCBD2 | YE | PDPN | RE | PI4KAP2 | MA | PLAC2 | BL | PLXNB2 |
| MA | PADI1 | GY | PCBP1 | BR | PDSS1 | GY | PI4KB | GY | PLAC8 | BR | PLXND1 |
| GY | PADI3 | YE | PCCA | RE | PDXDC2 | GN | PIAS3 | RE | PLAU | BR | PMEPA1 |
| GN | PAFAH1B3 | BR | PCDH12 | YE | PDZD2 | RE | PIF1 | MA | PLBD1 | BR | PMP22 |
| BR | PMS2L11 | MA | PPL | YE | PRKD1 | BR | PTK7 | GY | RAB12 | BL | RAPGEF1 |
| BR | PNCK | GY | PPM1F | GY | PRKY | YE | PTN | BL | RAB1A | MA | RAPGEF3 |
| BR | PNLDC1 | BL | PPM1K | YE | PRLR | BR | PTP4A3 | PI | RAB20 | MA | RAPGEFL1 |
| YE | PNMAL2 | BL | PPM1M | GN | PRMT10 | GN | PTPN12 | MA | RAB25 | BR | RARA |
| BL | PNO1 | BL | PPME1 | GY | PRNP | BL | PTPN22 | RE | RAB28 | MA | RARG |
| GN | PNPLA6 | BL | PPP1CB | GY | PROCR | GN | PTPN3 | RE | RAB2A | PI | RARRES1 |
| YE | PNPO | GY | PPP1R10 | YE | PRODH | BL | PTPN6 | YE | RAB35 | BR | RARRES2 |
| YE | PNRC1 | MA | PPP1R11 | MA | PROM2 | BL | PTPN7 | YE | RAB36 | GY | RARS2 |
| BL | PNRC2 | BR | PPP1R13B | YE | PROS1 | GN | PTPRA | BL | RAB37 | BR | RASA3 |
| BR | POC5 | MA | PPP1R13L | MA | PROSC | BL | PTPRCAP | BR | RAB3D | BL | RASA4P |
| BR | PODNL1 | GY | PPP1R14C | GN | PRPF19 | BL | PTPRC | BR | RAB3IL1 | BR | RASA4 |
| BR | PODN | BL | PPP1R16B | BR | PRPF38A | MA | PTPRH | GY | RAB3IP | MA | RASAL1 |
| YE | PODXL | BR | PPP1R3C | PI | PRPSAP1 | BL | PTPRJ | BL | RAB40B | BL | RASAL3 |
| BR | POLA2 | RE | PPP1R3E | BL | PRR15 | YE | PTPRM | GY | RAB40C | BR | RASD1 |
| GY | POLDIP3 | BL | PPP1R9B | YE | PRR5L | BL | PTPRN2 | YE | RAB42 | YE | RASD2 |
| BR | POLE3 | BR | PPP2CA | YE | PRRX1 | YE | PTPRS | RE | RAB5C | YE | RASGEF1A |
| RE | POLG2 | GY | PPP2CB | BR | PRSS16 | BL | PTPRU | GY | RAB7L1 | YE | RASGEF1B |
| GY | POLG | BL | PPP2R2B | BL | PRSS21 | BR | PTRF | BL | RAB8A | BL | RASGRP1 |
| BL | POLM | YE | PPP2R3A | MA | PRSS22 | GY | PTTG1IP | BL | RAB8B | BL | RASGRP2 |
| GN | POLR2E | YE | PPP2R5A | BR | PRSS23 | GN | PUS10 | YE | RAB9A | BL | RASGRP3 |
| GY | POLR2J2 | BL | PPP2R5B | MA | PRSS8 | BL | PVRIG | BL | RABGEF1 | BR | RASL12 |
| GN | POLR3D | GN | PPP3CA | BL | PRTFDC1 | MA | PVRL4 | RE | RABL2A | GY | RASSF10 |
| YE | POLR3G | BR | PPP3CB | PI | PSAP | BL | PVR | GN | RABL2B | BL | RASSF2 |
| GN | POLR3K | GN | PPP3CC | BR | PSAT1 | GN | PWP2 | BL | RAC2 | PI | RASSF4 |
| GN | POLRMT | BR | PPP4R1 | BL | PSD4 | BR | PXDN | GY | RAD1 | BL | RASSF5 |
| GN | PON2 | YE | PPP4R4 | RE | PSMC3IP | RE | PXN | GN | RAD23A | GN | RAVER1 |
| GY | PON3 | PI | PPT1 | BL | PSMD14 | YE | PYCARD | GN | RAD51C | BR | RBBP7 |
| GY | POP1 | PI | PQLC3 | BR | PSMD1 | BL | PYCR1 | MA | RAD51L3 | BR | RBM14 |
| GN | POP7 | GY | PRAME | BR | PSPC1 | YE | PYGL | BR | RAD54L | YE | RBM19 |
| BL | POR | BR | PRCP | BL | PSTPIP1 | BL | PYHIN1 | RE | RAD9A | GY | RBM23 |
| BR | POSTN | GN | PRDX2 | YE | PSTPIP2 | BL | ProSAPiP1 | BR | RAD9B | YE | RBM38 |
| BL | POU2AF1 | BR | PRELP | RE | PTBP2 | YE | QDPR | MA | RAET1E | BR | RBM39 |
| GY | POU2F1 | BL | PREX1 | YE | PTCD1 | PI | QPCT | MA | RAET1G | GN | RBM42 |
| BL | POU2F2 | BL | PRF1 | RE | PTCD3 | RE | QRICH2 | BL | RAET1L | BR | RBM45 |
| BL | POU6F1 | BR | PRICKLE1 | BR | PTENP1 | GY | QRSL1 | GY | RAF1 | RE | RBM5 |
| GN | PPAN | BR | PRICKLE2 | BR | PTEN | RE | QSOX1 | YE | RAI14 | RE | RBM6 |
| BR | PPAP2A | MA | PRICKLE4 | BL | PTGDS | RE | QSOX2 | BL | RAI2 | GN | RBMS1 |
| YE | PPAP2B | BL | PRIMA1 | BL | PTGER4 | RE | QTRT1 | MA | RALA | RE | RBMX |
| BL | PPAP2C | YE | PRKAB1 | GN | PTGES3 | MA | RAB10 | YE | RALB | BL | RBP1 |
| GN | PPARGC1A | YE | PRKAG2 | YE | PTGES | MA | RAB11A | RE | RALGPS1 | GN | RBP7 |
| BR | PPARG | BL | PRKAR2B | GY | PTGR2 | GN | RAB11B | YE | RAMP1 | MA | RBPJ |
| YE | PPFIBP2 | BL | PRKCB | BR | PTGS1 | BR | RAB11FIP3 | BL | RAMP3 | BR | RBPMS |
| BR | PPHLN1 | PI | PRKCQ | BL | PTK2B | YE | RAB11FIP4 | GN | RANBP3 | YE | RCAN1 |
| BR | PPIL3 | GN | PRKCSH | MA | PTK6 | BR | RAB11FIP5 | YE | RAP2B | BR | RCAN2 |
| BR | RCC1 | BR | RIBC2 | YE | ROR2 | BR | SAMD4A | GN | SDHA | PI | SERPING1 |
| BR | RCC2 | BL | RILPL2 | YE | RORB | BL | SAMD9L | YE | SDK1 | BR | SERPINH1 |
| GN | RCHY1 | GN | RIMS3 | RE | RPAIN | BL | SAMSN1 | YE | SDK2 | GN | SERTAD2 |
| GN | RCL1 | BL | RIN3 | YE | RPH3AL | YE | SAP30L | BR | SDPR | YE | SERTAD3 |
| GY | RCN2 | BL | RINL | YE | RPIA | RE | SAP30 | GY | SDR16C5 | BL | SESN1 |
| BR | RCN3 | GY | RIOK2 | GN | RPS15 | RE | SAR1B | PI | SDS | RE | SETD4 |
| GN | RCOR3 | GN | RIPK1 | GY | RPS28 | GY | SARM1 | BL | SEC14L2 | BR | SETD8 |
| BL | RCSD1 | BR | RIPK4 | GY | RPS4Y1 | BL | SASH3 | RE | SEC31A | BL | SETDB2 |
| GN | RDH13 | BL | RLTPR | BR | RPS6KA2 | GN | SAT1 | RE | SEC31B | YE | SEZ6L2 |
| YE | RDH16 | PI | RNASE1 | GY | RRAGD | GY | SBDSP1 | RE | SECISBP2 | BR | SF1 |
| BR | RECK | PI | RNASE6 | GY | RRAS2 | BL | SBDS | BL | SEL1L3 | GN | SF3A2 |
| RE | RECQL5 | BL | RNASEH1 | YE | RRAS | MA | SC4MOL | GY | SELENBP1 | BL | SF3B4 |
| GN | REEP4 | GN | RNASEH2A | BR | RRM2 | GN | SC5DL | BR | SELE | GN | SF4 |
| MA | REEP6 | YE | RND1 | GY | RRP15 | GN | SCAF1 | BL | SELL | RE | SFI1 |
| GY | RELA | MA | RND3 | RE | RRP7B | MA | SCAMP2 | BL | SELPLG | BR | SFPQ |
| YE | RELB | GN | RNF103 | GN | RRS1 | GN | SCAMP4 | BL | SELP | BR | SFRP1 |
| BL | RELT | BR | RNF122 | GY | RTCD1 | GN | SCAMP5 | BL | SEMA3B | BR | SFRP2 |
| PI | RENBP | BL | RNF125 | RE | RTEL1 | RE | SCAND2 | RE | SEMA3C | BR | SFRP4 |
| GN | REXO1 | GN | RNF126 | PI | RTN1 | BR | SCARA3 | MA | SEMA3F | RE | SFRS16 |
| GY | RFK | GN | RNF130 | BR | RTN3 | PI | SCARB1 | BR | SEMA3G | RE | SFRS17A |
| BR | RFPL1S | RE | RNF139 | YE | RTN4RL1 | GN | SCARB2 | BL | SEMA4A | GY | SFRS2B |
| BL | RFTN1 | BR | RNF145 | RE | RUFY3 | PI | SCARF1 | YE | SEMA4C | RE | SFRS2 |
| GN | RFXANK | YE | RNF150 | BR | RUNX2 | BR | SCARF2 | BL | SEMA4D | BR | SFRS4 |
| RE | RG9MTD3 | RE | RNF152 | BL | RUNX3 | BR | SCCPDH | YE | SEMA5A | RE | SFRS5 |
| YE | RGAG4 | BL | RNF157 | BL | RUSC1 | YE | SCD5 | YE | SEMA6A | RE | SFRS6 |
| YE | RGMA | YE | RNF165 | BR | RUSC2 | GY | SCFD1 | BR | SEMA6B | RE | SFRS7 |
| BL | RGS10 | PI | RNF166 | GN | RUVBL2 | GY | SCHIP1 | BR | SEMA6D | RE | SFRS8 |
| BR | RGS16 | GY | RNF185 | BL | RXRA | GN | SCIN | BL | SEMA7A | BR | SFT2D2 |
| BL | RGS19 | YE | RNF19A | YE | RYR3 | GN | SCLT1 | PI | SEPHS1 | GY | SFXN1 |
| BL | RGS1 | YE | RNF19B | MA | S100A12 | GY | SCMH1 | GN | SEPHS2 | GY | SFXN2 |
| BR | RGS2 | RE | RNF207 | MA | S100A14 | BR | SCML1 | BR | SEPN1 | BR | SFXN3 |
| BR | RGS3 | GY | RNF212 | MA | S100A16 | MA | SCNN1A | RE | SEPT7P2 | BR | SGCB |
| BR | RGS4 | BL | RNF213 | PI | S100A4 | GY | SCNN1B | GY | SERHL | BR | SGCD |
| BR | RGS5 | BR | RNF214 | MA | S100A8 | YE | SCNN1G | PI | SERPINA1 | BR | SGCE |
| MA | RHBDL2 | YE | RNF216 | MA | S100A9 | GN | SCO1 | BR | SERPINA3 | GY | SGEF |
| MA | RHCG | BR | RNF24 | BL | S100B | GN | SCOC | MA | SERPINB13 | BL | SGPP1 |
| RE | RHEBL1 | BR | RNF34 | BR | S1PR1 | YE | SCUBE2 | MA | SERPINB1 | RE | SGSM2 |
| BR | RHOBTB1 | BL | RNF39 | BL | S1PR2 | GN | SCYL3 | MA | SERPINB2 | GN | SGTA |
| YE | RHOBTB2 | YE | RNF44 | BL | S1PR4 | MA | SDC1 | MA | SERPINB3 | BL | SGTB |
| BL | RHOB | BR | RNF4 | GN | SAE1 | BR | SDC2 | MA | SERPINB4 | RE | SH2B1 |
| BL | RHOF | GY | RNLS | GN | SAFB2 | PI | SDC3 | MA | SERPINB5 | YE | SH2B3 |
| BL | RHOH | YE | ROBO1 | GN | SAFB | GY | SDC4 | BR | SERPINE1 | BL | SH2D1A |
| BR | RHOQ | YE | ROBO2 | GY | SALL2 | MA | SDCBP2 | YE | SERPINE2 | BL | SH2D2A |
| BR | RHOU | BR | ROBO4 | GN | SAMD1 | GY | SDCCAG8 | YE | SERPINF1 | GN | SH2D3A |
| BL | SH2D3C | BL | SLAMF1 | BR | SLC29A2 | GY | SLCO4A1 | BR | SOD3 | BR | SREBF1 |
| BL | SH3BGRL | BL | SLAMF6 | PI | SLC29A3 | GY | SLFN13 | GN | SOLH | RE | SRGAP3 |
| BR | SH3BP1 | BL | SLAMF7 | PI | SLC2A3 | YE | SLIT3 | BR | SORBS2 | PI | SRGN |
| BL | SH3BP2 | PI | SLAMF8 | PI | SLC2A5 | BR | SLMO2 | BR | SORCS2 | BL | SRP68 |
| MA | SH3BP5L | BL | SLA | PI | SLC2A6 | GY | SLU7 | BL | SORD | BR | SRPR |
| YE | SH3BP5 | BR | SLBP | BL | SLC2A9 | GN | SMAD1 | YE | SORL1 | BR | SRPX2 |
| GN | SH3GL1 | PI | SLC11A1 | PI | SLC31A2 | YE | SMAD7 | GY | SORT1 | BR | SRPX |
| BL | SH3KBP1 | GY | SLC12A4 | GY | SLC34A2 | MA | SMAGP | YE | SOX15 | YE | SRRM3 |
| BR | SH3RF3 | YE | SLC12A7 | BL | SLC35A2 | BL | SMAP2 | GY | SOX2 | RE | SRRT |
| YE | SH3TC1 | YE | SLC12A8 | GN | SLC35A4 | YE | SMARCA2 | GN | SOX9 | RE | SS18L1 |
| BR | SH3YL1 | MA | SLC15A2 | MA | SLC35C1 | GY | SMARCA4 | BL | SP140L | BR | SS18 |
| GN | SHANK2 | PI | SLC15A3 | BL | SLC35E2 | GY | SMARCAL1 | BL | SP140 | BR | SSC5D |
| BR | SHANK3 | BR | SLC15A4 | YE | SLC35F2 | GY | SMARCD1 | GY | SPAG16 | MA | SSH3 |
| BR | SHC1 | GY | SLC16A14 | GY | SLC37A1 | BR | SMC1B | GN | SPAG1 | YE | SSPN |
| BR | SHC2 | BR | SLC16A2 | BR | SLC38A5 | BR | SMNDC1 | BR | SPARCL1 | BL | SSRP1 |
| YE | SHCBP1 | BL | SLC16A5 | PI | SLC38A6 | BR | SMOC2 | BR | SPARC | BR | ST3GAL2 |
| BR | SHE | BL | SLC17A9 | GN | SLC39A11 | GY | SMOX | GY | SPATA20 | BL | ST3GAL5 |
| YE | SHISA2 | BL | SLC19A2 | BR | SLC39A14 | BR | SMO | GN | SPATA5L1 | BR | ST3GAL6 |
| BR | SHMT1 | GN | SLC1A1 | MA | SLC39A2 | BR | SMPD4 | BR | SPC24 | GY | ST5 |
| GN | SHMT2 | PI | SLC1A3 | GN | SLC39A3 | YE | SMPDL3A | BR | SPC25 | BL | ST6GAL1 |
| YE | SHOX2 | GN | SLC1A5 | GN | SLC39A8 | GY | SMPDL3B | GN | SPCS3 | BR | ST6GAL2 |
| PI | SHROOM3 | PI | SLC20A1 | BR | SLC41A2 | BL | SMTN | GY | SPESP1 | GY | ST6GALNAC2 |
| BR | SHROOM4 | BL | SLC20A2 | BL | SLC43A2 | GN | SMU1 | GN | SPG20 | BL | ST7L |
| RE | SIAE | MA | SLC22A15 | GY | SLC44A2 | BR | SMYD2 | PI | SPI1 | BL | ST7 |
| BL | SIDT1 | BR | SLC24A3 | BL | SLC44A3 | BR | SNAI2 | YE | SPIB | YE | ST8SIA1 |
| BL | SIDT2 | GN | SLC25A10 | GY | SLC44A4 | RE | SNAPC4 | GN | SPIN3 | BL | ST8SIA4 |
| PI | SIGLEC10 | GY | SLC25A12 | BR | SLC45A3 | BR | SNCAIP | YE | SPIRE1 | PI | STAB1 |
| PI | SIGLEC1 | BL | SLC25A13 | YE | SLC45A4 | BR | SND1 | GN | SPIRE2 | RE | STAG3L3 |
| GN | SIGMAR1 | GY | SLC25A16 | PI | SLC46A3 | BR | SNED1 | GN | SPNS1 | BR | STAG3 |
| YE | SIK1 | GN | SLC25A19 | PI | SLC47A1 | RE | SNHG10 | BL | SPN | BL | STAMBPL1 |
| YE | SIM2 | YE | SLC25A22 | GY | SLC4A2 | RE | SNHG12 | BR | SPOCK1 | BL | STAMBP |
| BL | SIPA1 | BR | SLC25A23 | MA | SLC6A14 | RE | SNHG1 | BL | SPOCK2 | GN | STAP2 |
| PI | SIRPA | GY | SLC25A25 | GY | SLC6A15 | GN | SNN | BR | SPON1 | BR | STARD13 |
| PI | SIRPB1 | RE | SLC25A35 | YE | SLC6A8 | GN | SNRNP25 | BR | SPON2 | GY | STARD3NL |
| BL | SIRPG | BR | SLC25A37 | YE | SLC7A2 | GY | SNRNP27 | GY | SPOP | BR | STARD8 |
| GN | SIRT6 | GN | SLC25A3 | YE | SLC7A5 | RE | SNRNP70 | PI | SPP1 | YE | STAR |
| BL | SIT1 | GN | SLC25A42 | PI | SLC7A7 | BR | SNTB1 | GN | SPPL2B | BL | STAT4 |
| BL | SIX1 | GN | SLC25A43 | BR | SLC7A8 | BR | SNW1 | BR | SPRY4 | BL | STAT5A |
| GN | SIX2 | BL | SLC25A45 | PI | SLC8A1 | PI | SNX10 | YE | SPSB1 | BR | STAT5B |
| BR | SKA1 | YE | SLC26A9 | YE | SLC9A2 | BL | SNX20 | BL | SPTBN2 | BL | STC2 |
| BR | SKA2 | BL | SLC27A2 | MA | SLC9A3R1 | BL | SNX2 | RE | SPTBN5 | GN | STEAP3 |
| BR | SKA3 | YE | SLC27A4 | YE | SLC9A9 | MA | SNX33 | BR | SPTLC3 | BL | STIP1 |
| BL | SKAP1 | GN | SLC27A5 | YE | SLCO2A1 | GY | SNX3 | YE | SQSTM1 | BL | STK10 |
| BL | SLA2 | BR | SLC29A1 | PI | SLCO2B1 | BL | SOCS2 | MA | SRD5A3 | GN | STK11 |
| BL | STK17A | BR | SYCP2 | BR | TBX2 | YE | TGFB2 | YE | TINAGL1 | BR | TMEM171 |
| BL | STK17B | BR | SYDE1 | GY | TBX3 | BR | TGFB3 | BR | TIPIN | YE | TMEM173 |
| RE | STK36 | BL | SYK | MA | TBX6 | BR | TGFBI | GY | TJP3 | PI | TMEM176A |
| BR | STK40 | BR | SYNE1 | PI | TBXAS1 | BR | TGFBR2 | GN | TK1 | PI | TMEM176B |
| BL | STK4 | YE | SYNGR2 | GN | TC2N | BL | TGIF1 | PI | TLE1 | GN | TMEM180 |
| GN | STOML2 | BR | SYNGR3 | BR | TCAM1P | YE | TGIF2 | GY | TLE2 | MA | TMEM184A |
| GY | STOM | RE | SYNPO | GN | TCEA1 | BR | TGM2 | BL | TLK2 | RE | TMEM184B |
| YE | STON1 | BL | SYT11 | BR | TCF19 | YE | TG | RE | TLN1 | BR | TMEM200A |
| YE | STON2 | GY | SYT17 | GN | TCF3 | BR | THADA | BR | TLN2 | BR | TMEM201 |
| GY | STOX1 | BL | SYT7 | BR | TCF4 | BR | THAP10 | BL | TLR10 | BR | TMEM204 |
| GY | STRA6 | YE | SYTL3 | YE | TCF7L1 | GN | THAP2 | YE | TLR1 | YE | TMEM20 |
| BR | STRAP | BL | SYTL4 | GN | TCF7L2 | GN | THAP6 | PI | TLR4 | BR | TMEM214 |
| GN | STRN4 | BL | SYVN1 | BL | TCF7 | YE | THAP8 | YE | TLR6 | GY | TMEM220 |
| BR | STT3A | GN | TACO1 | GN | TCFL5 | GY | THBD | BL | TLR7 | BL | TMEM229B |
| GN | STX10 | MA | TACSTD2 | RE | TCHP | BR | THBS1 | PI | TLR8 | GY | TMEM22 |
| PI | STX11 | RE | TAF1C | BL | TCL1A | BR | THBS2 | BR | TLX3 | MA | TMEM231 |
| RE | STX16 | GY | TAF6 | MA | TCN1 | BR | THBS3 | MA | TM4SF1 | BR | TMEM2 |
| GN | STX2 | GY | TAF7L | PI | TCN2 | BL | THEM4 | PI | TM6SF1 | MA | TMEM40 |
| BL | STX3 | BR | TAF7 | BR | TCOF1 | BL | THEMIS | YE | TM7SF3 | BR | TMEM41B |
| YE | STXBP1 | BL | TAGAP | BL | TCP11 | GY | THNSL2 | GY | TM9SF1 | GY | TMEM45A |
| GN | STXBP2 | BR | TAGLN | GY | TCP1 | RE | THOC1 | BL | TMBIM1 | BR | TMEM47 |
| YE | STXBP6 | GN | TANK | YE | TCTN1 | BR | THOC3 | MA | TMC4 | BR | TMEM55A |
| BR | SULF1 | BL | TAPBPL | GY | TCTN3 | GN | THOP1 | BL | TMC8 | GY | TMEM56 |
| BR | SULF2 | GY | TAPT1 | YE | TDRD10 | BL | THRB | YE | TMCC3 | GY | TMEM57 |
| RE | SULT1A3 | BR | TARDBP | GY | TDRD5 | RE | THSD1 | BL | TMCO1 | GY | TMEM5 |
| YE | SULT1E1 | BL | TARS2 | GY | TDRKH | GN | THSD4 | BR | TMCO4 | GN | TMEM63A |
| MA | SULT2B1 | YE | TARSL2 | MA | TEAD3 | RE | THUMPD2 | RE | TMCO6 | BL | TMEM66 |
| MA | SUMF1 | GY | TARS | GN | TECR | GY | THUMPD3 | GN | TMED1 | GY | TMEM68 |
| MA | SUOX | GN | TATDN1 | GY | TEF | BR | THY1 | GY | TMEM104 | BR | TMEM69 |
| GY | SUPT3H | GN | TBC1D10B | BR | TEK | BR | TIAL1 | BL | TMEM106A | MA | TMEM79 |
| GN | SUPT5H | BL | TBC1D10C | BR | TENC1 | BR | TIAM2 | GN | TMEM109 | PI | TMEM86A |
| RE | SUPT7L | BR | TBC1D16 | YE | TESC | MA | TICAM1 | YE | TMEM117 | BR | TMEM98 |
| BR | SUSD2 | BR | TBC1D1 | GN | TFAP2A | BR | TIE1 | BR | TMEM119 | GN | TMEM99 |
| BL | SUSD3 | RE | TBC1D3B | YE | TFAP2C | BR | TIFA | BL | TMEM140 | MA | TMPRSS11A |
| GY | SUSD4 | RE | TBC1D3 | GN | TFAP4 | RE | TIGD1 | GN | TMEM143 | MA | TMPRSS11D |
| BR | SUV39H1 | GN | TBCK | BR | TFB1M | GN | TIGD2 | BL | TMEM149 | GY | TMPRSS2 |
| BR | SUV39H2 | YE | TBKBP1 | BL | TFB2M | GY | TIGD6 | BL | TMEM14A | BL | TMPRSS4 |
| RE | SUV420H2 | YE | TBL1X | MA | TFCP2L1 | BL | TIGIT | YE | TMEM150C | PI | TMSB15A |
| RE | SUZ12P | GN | TBL2 | PI | TFEC | GN | TIMM13 | MA | TMEM154 | BR | TMTC1 |
| YE | SV2B | GY | TBPL1 | BL | TFG | GN | TIMM44 | MA | TMEM159 | RE | TMUB2 |
| RE | SVIL | GY | TBP | BR | TFPI | GN | TIMM50 | GN | TMEM160 | BR | TMX4 |
| GY | SVIP | PI | TBRG1 | GN | TGDS | BR | TIMP1 | GN | TMEM161A | BR | TNC |
| BL | SYAP1 | BR | TBX15 | GN | TGFA | BR | TIMP2 | MA | TMEM165 | YE | TNFAIP2 |
| GY | SYBU | BR | TBX1 | BR | TGFB1I1 | BR | TIMP3 | BR | TMEM170B | GN | TNFAIP3 |
| BR | TNFAIP6 | GY | TPMT | GY | TRPM4 | YE | TUBB2B | GN | UFM1 | GN | VDAC3 |
| BL | TNFAIP8L2 | PI | TPP1 | RE | TRPV1 | BR | TUBD1 | GY | UGT1A6 | GN | VEGFA |
| BL | TNFAIP8 | BL | TPPP3 | PI | TRPV2 | GY | TUBE1 | RE | UHRF2 | BR | VEGFC |
| GN | TNFRSF10D | BL | TPPP | BR | TRPV4 | BL | TUBG1 | BL | ULBP2 | BR | VGLL3 |
| BL | TNFRSF12A | MA | TPRXL | YE | TRPV6 | RE | TUBGCP6 | GY | ULK1 | GY | VILL |
| BL | TNFRSF14 | BR | TPSAB1 | YE | TSC22D1 | GN | TUFM | PI | UNC13D | PI | VIM |
| BL | TNFRSF17 | BR | TPSB2 | YE | TSC2 | YE | TUSC3 | GN | UNC45A | MA | VNN1 |
| YE | TNFRSF19 | BL | TPST1 | YE | TSEN15 | BR | TWIST1 | GN | UNC5B | BL | VNN2 |
| BL | TNFRSF1A | RE | TRA2A | GY | TSGA14 | BL | TXNDC11 | GY | UNG | PI | VOPP1 |
| BL | TNFRSF1B | BL | TRAF1 | BL | TSKU | BL | TXNDC5 | BL | UNKL | BL | VPS11 |
| RE | TNFRSF25 | YE | TRAF2 | GY | TSLP | BL | TXNIP | RE | UNK | GY | VPS26B |
| BL | TNFRSF4 | BL | TRAF3IP3 | GN | TSNAX | BL | TXNRD3IT1 | GN | UPF1 | MA | VPS37B |
| YE | TNFRSF6B | BL | TRAF5 | BL | TSN | YE | TYK2 | RE | UPF3A | GY | VPS37C |
| YE | TNFRSF9 | BL | TRAFD1 | BR | TSPAN11 | RE | TYMS | GY | UPK1B | YE | VRK2 |
| PI | TNFSF12.TN [symbol] | [symbol] | TRANK1 | BR | TSPAN12 | YE | TYRO3 | BR | UQCC | RE | VSIG10 |
| PI | TNFSF12 | GN | TRAP1 | GY | TSPAN13 | PI | TYROBP | GN | UQCR11 | PI | VSIG4 |
| BL | TNFSF13B | GN | TRAPPC5 | YE | TSPAN17 | BR | U2AF2 | GN | UQCRC2 | YE | VSTM2L |
| PI | TNFSF13 | BR | TRAPPC6B | BR | TSPAN18 | BL | UAP1 | GN | USE1 | BL | VTCN1 |
| MA | TNFSF4 | GY | TRDMT1 | BL | TSPAN1 | RE | UBA1 | BL | USH1G | BR | VWA5A |
| GN | TNFSF9 | PI | TREM2 | BL | TSPAN33 | BL | UBA7 | MA | USP11 | BR | VWF |
| BL | TNF | BR | TRIM13 | BL | TSPAN3 | BL | UBASH3A | GY | USP21 | GY | WARS2 |
| YE | TNIP1 | BL | TRIM14 | PI | TSPAN4 | BL | UBASH3B | GN | USP27X | RE | WASH7P |
| BR | TNK1 | YE | TRIM16L | BR | TSPAN7 | YE | UBD | BR | USP39 | BL | WAS |
| BL | TNKS1BP1 | MA | TRIM16 | YE | TSPAN9 | GY | UBE2D3 | BL | USP43 | BL | WBP5 |
| YE | TNS3 | GN | TRIM28 | RE | TSPYL2 | BR | UBE2D4 | GY | USP5 | YE | WBSCR17 |
| BL | TNS4 | MA | TRIM29 | BR | TSPYL5 | RE | UBE2G2 | GY | USP9Y | BL | WDFY4 |
| GY | TOMM20 | BL | TRIM38 | GY | TTC12 | BL | UBE2J1 | BL | USPL1 | GN | WDR18 |
| GN | TOMM40 | GY | TRIM3 | MA | TTC22 | GY | UBE2N | GN | UTP18 | YE | WDR19 |
| BL | TOMM70A | GY | TRIM45 | GY | TTC23 | YE | UBE2Q2 | YE | UXS1 | RE | WDR27 |
| RE | TOP3B | YE | TRIM47 | BR | TTC31 | YE | UBE2QL1 | BL | VAMP1 | GY | WDR33 |
| BL | TOX2 | BR | TRIM59 | BL | TTC39A | GN | UBE2R2 | MA | VAMP3 | BL | WDR41 |
| BL | TOX | GN | TRIM65 | BL | TTC39C | GN | UBE2V2 | GY | VAMP4 | BL | WDR45L |
| BL | TP53AIP1 | GN | TRIM68 | GY | TTC7A | GY | UBIAD1 | PI | VAMP5 | RE | WDR62 |
| YE | TP53I11 | BL | TRIM7 | MA | TTC9 | GN | UBL5 | GN | VANGL2 | BL | WDR72 |
| BL | TP53I3 | YE | TRIM8 | RE | TTF1 | GY | UBLCP1 | BR | VAPA | RE | WDR73 |
| BL | TP53INP1 | BL | TRIP13 | MA | TTLL12 | BR | UBTD1 | BL | VASH1 | GY | WDR75 |
| BL | TP53INP2 | MA | TRIP4 | RE | TTLL3 | YE | UBTF | YE | VASH2 | BL | WDR81 |
| YE | TP73 | GY | TRMT12 | GN | TTLL7 | RE | UBXN11 | BR | VASN | RE | WDR85 |
| BL | TPBG | RE | TRMT1 | GY | TTL | BL | UBXN2A | BL | VAV1 | RE | WDR90 |
| BR | TPCN1 | MA | TRNP1 | GY | TTTY15 | GN | UBXN6 | YE | VAV2 | YE | WDR91 |
| GY | TPCN2 | GY | TRNT1 | PI | TTYH2 | GN | UBXN8 | YE | VCAM1 | BR | WDSUB1 |
| YE | TPD52L1 | RE | TROAP | BR | TTYH3 | GY | UCHL1 | BR | VCAN | GY | WFDC2 |
| BR | TPM1 | BR | TRO | BR | TUBA1A | BL | UCK2 | GY | VCP | BR | WFS1 |
| BR | TPM2 | PI | TRPM2 | BL | TUBB2A | BL | UCP2 | GN | VDAC1 | BL | WHAMM |
| BR | WHSC1 | BL | ZBP1 | MA | ZNF251 | BL | ZNF502 | MA | ZNF750 | | |
| BR | WHSC2 | BL | ZBTB24 | BL | ZNF253 | GY | ZNF503 | GY | ZNF764 | | |
| BL | WIPF1 | GR | ZBTB3 | GY | ZNF256 | BL | ZNF506 | GY | ZNF766 | | |
| BR | WIPI1 | GY | ZBTB42 | GR | ZNF25 | GY | ZNF512B | RE | ZNF767 | | |
| BR | WISP1 | GR | ZBTB45 | GR | ZNF263 | YE | ZNF512 | GR | ZNF777 | | |
| YE | WNK2 | YE | ZBTB46 | RE | ZNF266 | RE | ZNF513 | BR | ZNF77 | | |
| YE | WNT10A | RE | ZBTB49 | GY | ZNF271 | BR | ZNF521 | RE | ZNF785 | | |
| YE | WNT10B | MA | ZBTB7B | GY | ZNF273 | BL | ZNF526 | GR | ZNF787 | | |
| YE | WNT2B | BR | ZC3H8 | BR | ZNF274 | BL | ZNF527 | RE | ZNF789 | | |
| BR | WNT2 | BR | ZCCHC10 | RE | ZNF276 | GR | ZNF528 | BL | ZNF793 | | |
| PI | WNT3A | BR | ZCCHC24 | GR | ZNF282 | GY | ZNF529 | GY | ZNF799 | | |
| YE | WNT4 | GY | ZCCHC7 | BR | ZNF287 | BR | ZNF541 | BL | ZNF79 | | |
| BR | WNT5A | BR | ZCCHC9 | BL | ZNF2 | BR | ZNF542 | BL | ZNF814 | | |
| YE | WNT5B | BR | ZDHHC13 | BL | ZNF300 | GY | ZNF544 | BR | ZNF823 | | |
| BR | WRNIP1 | BL | ZDHHC1 | MA | ZNF323 | BL | ZNF549 | BR | ZNF830 | | |
| RE | WSB1 | BR | ZDHHC23 | GY | ZNF324 | BR | ZNF552 | BL | ZNF831 | | |
| RE | WSB2 | BR | ZDHHC2 | GY | ZNF329 | GR | ZNF554 | RE | ZNF839 | | |
| YE | WSCD1 | BR | ZDHHC6 | GR | ZNF330 | BL | ZNF557 | RE | ZNF83 | | |
| GY | WTAP | YE | ZDHHC9 | RE | ZNF335 | GR | ZNF564 | GR | ZNF841 | | |
| BL | WWC1 | BR | ZEB1 | RE | ZNF337 | BL | ZNF566 | BR | ZNF853 | | |
| GR | WWC2 | BR | ZEB2 | GR | ZNF341 | BL | ZNF569 | BL | ZNF879 | | |
| GR | WWC3 | GY | ZFAND1 | GR | ZNF343 | GR | ZNF574 | GR | ZNRF2 | | |
| YE | WWOX | YE | ZFP112 | BL | ZNF350 | BL | ZNF577 | BR | ZSCAN16 | | |
| GR | XAB2 | GY | ZFP36L2 | GR | ZNF358 | BR | ZNF57 | | | | |
| BL | XBP1 | GR | ZFPM1 | GY | ZNF362 | BL | ZNF585A | | | | |
| YE | XG | GR | ZFPM2 | BL | ZNF383 | GY | ZNF586 | | | | |
| GY | XK | BR | ZFR2 | GY | ZNF385A | BR | ZNF595 | | | | |
| BL | XPC | BL | ZFYVE28 | GR | ZNF397OS | GR | ZNF598 | | | | |
| YE | XPNPEP1 | GY | ZFY | GR | ZNF3 | BL | ZNF600 | | | | |
| GY | YAF2 | MA | ZG16B | GR | ZNF414 | BL | ZNF607 | | | | |
| GR | YARS2 | YE | ZMIZ2 | BL | ZNF416 | GY | ZNF613 | | | | |
| GR | YBX1 | BL | ZNF101 | BL | ZNF419 | GR | ZNF628 | | | | |
| BR | YBX2 | GR | ZNF117 | BR | ZNF423 | GY | ZNF629 | | | | |
| BR | YEATS4 | BL | ZNF14 | GY | ZNF425 | GR | ZNF638 | | | | |
| GR | YIPF2 | GY | ZNF155 | GY | ZNF438 | GR | ZNF653 | | | | |
| GY | YIPF4 | MA | ZNF165 | BL | ZNF43 | BR | ZNF675 | | | | |
| RE | YJEFN3 | GR | ZNF175 | BL | ZNF441 | BL | ZNF683 | | | | |
| PI | YOD1 | MA | ZNF185 | GY | ZNF443 | RE | ZNF692 | | | | |
| GR | YPEL2 | BL | ZNF187 | BR | ZNF467 | RE | ZNF700 | | | | |
| GY | YRDC | BL | ZNF211 | BR | ZNF469 | GR | ZNF706 | | | | |
| BL | YWHAQ | BR | ZNF234 | GR | ZNF480 | GY | ZNF711 | | | | |
| BL | YWHAZ | BL | ZNF235 | YE | ZNF488 | BR | ZNF721 | | | | |
| BL | ZAP70 | YE | ZNF238 | GR | ZNF48 | BL | ZNF738 | | | | |
| YE | ZBED1 | RE | ZNF248 | BL | ZNF490 | BL | ZNF74 | | | | |

WG = WGCNA, Blue = BL, Brown = BR, Green = GN, Grey = GY, Magenta = MA, Pink = PI, Red = RE, Yellow = YE. [symbol]
indicates data missing or illegible when filed

TABLE 4
Hypergeometric enrichment analysis comparing WGCNA modules and MISigDB Hallmark Gene Sets. Adjusted P-values are as produced
from EnrichR R package. Ratio represents the number of Hallmark gene set genes are members of the inticated WGCNA module.

| | p. adjust_{—} | p. adjust_{—} | p. adjust_{—} | p. adjust_{—} | p. adjust_{—} | p. adjust_{—} | p. adjust_{—} |
|---|---|---|---|---|---|---|---|
| Description | blue | bro [symbol] | yello [symbol] | gre [symbol] | re [symbol] | pi [symbol] | mage |
| HALLMARK_ALLOGRAFT_REJECTION | 1.76E−15 | | | | | 0.0791371 [symbol] | |
| HALLMARK_INTERFERON_GAMMA_{—} | 1.42E−06 | | | | | 0.072368 [symbol] | |
| RESPO [symbol] | | | | | | | |
| HALLMARK_IL2_STAT5_SIGNALING | 5.27E−05 | | | | | | |
| HALLMARK_IL6_JAK_STAT3_{—} | 9.10E−04 | | | | | | |
| SIGNALING | | | | | | | |
| HALLMARK_INTERFERON_ALPHA_{—} | 0.021450565 [symbol] | | | | | | |
| RESPON [symbol] | | | | | | | |
| HALLMARK_INFLAMMATORY_{—} | 0.045898895 [symbol] | | | | | 0.0077113 [symbol] | |
| RESPONSE | | | | | | | |
| HALLMARK_COMPLEMENT | 0.074558502 [symbol] | | | | | 0.0073101 [symbol] | |
| HALLMARK_EPITHELIAL_{—} | | 1.14E− [symbol] | | | | | |
| MESENCHYMAL_T [symbol] | | | | | | | |
| HALLMARK_MYOGENESIS | | 1.18E− [symbol] | | | | | |
| HALLMARK_G2M_CHECKPOINT | | 4.30E− [symbol] | | | | | |
| HALLMARK_UV_RESPONSE_DN | | 0.0012603 [symbol] | | | | | |
| HALLMARK_E2F_TARGETS | | 0.0027778 [symbol] | | | | | |
| HALLMARK_COAGULATION | | 0.0035163 [symbol] | | | | 0.0077113 [symbol] | |
| HALLMARK_SPERMATOGENESIS | | 0.0156214 [symbol] | | | | | |
| HALLMARK_ANGIOGENESIS | | 0.0304179 [symbol] | | | | | |
| HALLMARK_APICAL_JUNCTION | | 0.0314497 [symbol] | | | | | |
| HALLMARK_TNFA_SIGNALING_VIA_{—} | | | 6.30E− [symbol] | | | | |
| NFKB | | | | | | | |
| HALLMARK_ESTROGEN_RESPONSE_{—} | | | 0.0901457 [symbol] | | | | |
| EARLY | | | | | | | |
| HALLMARK_OXIDATIVE_{—} | | | | 1.07E− [symbol] | | | |
| PHOSPHORYLATIO [symbol] | | | | | | | |
| HALLMARK_MYC_TARGETS_V1 | | | | 3.38E− [symbol] | | | |
| HALLMARK_MYC_TARGETS_V2 | | | | 7.43E− [symbol] | | | |
| HALLMARK_ADIPOGENESIS | | | | 0.0065944 [symbol] | | | |
| HALLMARK_DNA_REPAIR | | | | | 0.0979634 [symbol] | | |
| HALLMARK_UNFOLDED_PROTEIN_{—} | | | | | 0.0979634 [symbol] | | |
| RESPON [symbol] | | | | | | | |
| HALLMARK_KRAS_SIGNALING_UP | | | | | | 0.0265877 [symbol] | |
| HALLMARK_ESTROGEN_RESPONSE_{—} | | | | | | | 0.0054382 [symbol] |
| LATE | | | | | | | |
| HALLMARK_KRAS_SIGNALING_DN | | | | | | | 0.0570496 [symbol] |
| HALLMARK_P53_PATHWAY | | | | | | | 0.0570496 [symbol] |

| | Ratio_{—} | Ratio_{—} | Ratio_{—} | Ratio_{—} | Ratio_{—} | Ratio_{—} | Ratio_{—} |
|---|---|---|---|---|---|---|---|
| Description | blue | brown | yellow | green | red | pink | mage |
| HALLMARK_ALLOGRAFT_REJECTION | 0.06293706 [symbol] | | | | | 0.0526315 [symbol] | |
| HALLMARK_INTERFERON_GAMMA_{—} | 0.03796203 [symbol] | | | | | 0.0421052 [symbol] | |
| RESPO [symbol] | | | | | | | |
| HALLMARK_IL2_STAT5_SIGNALING | 0.04195804 [symbol] | | | | | | |
| HALLMARK_IL6_JAK_STAT3_{—} | 0.02197802 [symbol] | | | | | | |
| SIGNALING | | | | | | | |
| HALLMARK_INTERFERON_ALPHA_{—} | 0.01198801 [symbol] | | | | | | |
| RESPON [symbol] | | | | | | | |
| HALLMARK_INFLAMMATORY_{—} | 0.03296703 [symbol] | | | | | 0.0596491 [symbol] | |
| RESPONSE | | | | | | | |
| HALLMARK_COMPLEMENT | 0.03196803 [symbol] | | | | | 0.0631578 [symbol] | |
| HALLMARK_EPITHELIAL_{—} | | 0.095472 [symbol] | | | | | |
| MESENCHYMAL_T [symbol] | | | | | | | |
| HALLMARK_MYOGENESIS | | 0.034448 [symbol] | | | | | |
| HALLMARK_G2M_CHECKPOINT | | 0.026574 [symbol] | | | | | |
| HALLMARK_UV_RESPONSE_DN | | 0.027559 [symbol] | | | | | |
| HALLMARK_E2F_TARGETS | | 0.025590 [symbol] | | | | | |
| HALLMARK_COAGULATION | | 0.027559 [symbol] | | | | 0.0456140 [symbol] | |
| HALLMARK_SPERMATOGENESIS | | 0.012795 [symbol] | | | | | |
| HALLMARK_ANGIOGENESIS | | 0.011811 [symbol] | | | | | |
| HALLMARK_APICAL_JUNCTION | | 0.030511 [symbol] | | | | | |
| HALLMARK_TNFA_SIGNALING_VIA_{—} | | | 0.05372617 [symbol] | | | | |
| NFKB | | | | | | | |
| HALLMARK_ESTROGEN_RESPONSE_{—} | | | 0.036395147 [symbol] | | | | |
| EARLY | | | | | | | |
| HALLMARK_OXIDATIVE_{—} | | | | 0.0416666 [symbol] | | | |
| PHOSPHORYLATIO [symbol] | | | | | | | |
| HALLMARK_MYC_TARGETS_V1 | | | | 0.0288461 [symbol] | | | |
| HALLMARK_MYC_TARGETS_V2 | | | | 0.0160256 [symbol] | | | |
| HALLMARK_ADIPOGENESIS | | | | 0.027243 [symbol] | | | |
| HALLMARK_DNA_REPAIR | | | | | 0.0136239 [symbol] | | |
| HALLMARK_UNFOLDED_PROTEIN_{—} | | | | | 0.0163487 [symbol] | | |
| RESPON [symbol] | | | | | | | |
| HALLMARK_KRAS_SIGNALING_UP | | | | | | 0.0561403 [symbol] | |
| HALLMARK_ESTROGEN_RESPONSE_{—} | | | | | | | 0.049 [symbol] |
| LATE | | | | | | | |
| HALLMARK_KRAS_SIGNALING_DN | | | | | | | 0.028 [symbol] |
| HALLMARK_P53_PATHWAY | | | | | | | 0.038 [symbol] |

[symbol]

indicates data missing or illegible when filed

TABLE 5
Clinical characteristics of Vanderbilt
cohort of HPV + HNSCC patients

| | | NFkB | NFkB | |
|---|---|---|---|---|
| | | Inactive | Active | |
| | | n = 52 | n = 41 | p-value |
| Pathologic N Stage (%) | N0 | 3 (13.0) | 3 (15.8) | 0.31 |
| | N1 | 7 (30.4) | 2 (10.5) | |
| | N2 | 12 (52.2) | 14 (73.7) | |
| | N3 | 1 (4.3) | 0 (0.0) | |
| Pathologic T Stage (%) | T0 | 1 (4.3) | 2 (9.5) | 0.152 |
| | T1 | 13 (56.5) | 14 (66.7) | |
| | T2 | 9 (39.1) | 3 (14.3) | |
| | T3 | 0 (0.0) | 2 (9.5) | |
| Pathologic Summary | | | | |
| Stage (%) | Stage 1 | 1 (5.3) | 0 (0.0) | 0.773 |
| | Stage 2 | 2 (10.5) | 1 (6.7) | |
| | Stage 3 | 3 (15.8) | 2 (13.3) | |
| | Stage 4 | 13 (68.4) | 12 (80.0) | |
| Treatment Strategy(%) | S | 6 (12.0) | 3 (7.5) | 0.334 |
| | S + CXRT | 21 (42.0) | 23 (57.5) | |
| | CXRT | 23 (46.0) | 14 (35.0) | |
| Race (%) | Other | 0 (0.0) | 2 (4.9) | 0.373 |
| | White | 52 (100.0) | 39 (95.1) | |
| Sex (%) | F | 2 (3.8) | 5 (12.2) | 0.263 |
| | M | 50 (96.2) | 36 (87.8) | |
| | Never | | | |
| Smoking (%) | Smoker | 17 (32.7) | 17 (42.5) | 0.454 |
| | Smoker | 35 (67.3) | 23 (57.5) | |
| Age (%) | <50 | 16 (30.8) | 8 (19.5) | 0.321 |
| | >=50 | 36 (69.2) | 33 (80.5) | |

XRT: Radiation Therapy
CXRT: Chemoradiation Therapy
S: Surgery

1. A method for evaluating the prognosis of a human papilloma virus (HPV) associated head and neck cancer patient, comprising detecting defects in nucleic acids encoding genes, or their expression products, of TRAF3, CYLD, TRAF2, MYD88, NFKBIA, TNFAIP3, TRAF6, BIRC2, BIRC3, and MAP3K14 in a sample from the patient, normalized against a reference set of nucleic acids encoding genes, or their expression products, in the sample, wherein defects in the nucleic acids or their expression products is indicative of prognosis, thereby evaluating the prognosis of the head and neck cancer patient.

2. The method of claim 1, wherein the head and neck cancer is an oropharyngeal squamous cell carcinoma (OPSCC), a nasopharyngeal squamous cell carcinoma, a squamous cell carcinomas of the nasal cavity or paranasal sinuses, a squamous cell carcinoma of the oral cavity, or a squamous cell carcinoma of the hypopharynx.

3. The method of claim 3, wherein the head and neck cancer is an oropharyngeal squamous cell carcinoma (OPSCC).

4. (canceled)

5. (canceled)

6. The method of claim 1, wherein the defects are mutations or copy number alterations.

7. The method of claim 6, wherein the mutations are missense mutations, nonsense mutations, frameshift mutations, insertions, and/or deletions.

8. The method of claim 1, wherein the detecting defects in nucleic acids encoding genes, or their expression products, for the biomarkers comprises performing next generation sequencing (NGS), nucleic acid hybridization, quantitative RT-PCR, or immunohistochemistry (IHC), immunocytochemistry (ICC), or immunofluorescence (IF).

9. The method of claim 1, wherein the method for evaluating the prognosis of a head and neck cancer patient further comprises assessment of a medical history, a family history, a physical examination, an endoscopic examination, imaging, a biopsy result, or a combination thereof.

10. The method of claim 10, wherein the method is used to develop a treatment strategy for the head and neck cancer patient.

11. The method of claim 1, wherein the nucleic acids encoding genes are isolated from a fixed, paraffin-embedded sample from the patient.

12. The method of claim 1, wherein the nucleic acids encoding genes are isolated from core biopsy tissue or fine needle aspirate cells from the patient.

13. A method for predicting a response of a human papilloma virus (HPV) associated head and neck cancer patient to a selected treatment, comprising detecting defects in nucleic acids encoding genes, or their expression products, of TRAF3, CYLD, TRAF2, MYD88, NFKBIA, TNFAIP3, TRAF6, BIRC2, BIRC3, and MAP3K14 in a sample from the patient, normalized against a reference set of nucleic acids encoding genes, or their expression products, in the sample, wherein defects in the nucleic acids, or their expression products, is indicative of a positive treatment response, thereby predicting the response of the head and cancer patient to the treatment.

14. (canceled)

15. (canceled)

16. (canceled)

17. (canceled)

18. (canceled)

19. (canceled)

20. A method for evaluating the prognosis of a human papilloma virus (HPV) associated head and neck cancer patient, comprising measuring mRNA expression of MGAT3, STAR, VCAM1, RAB42, NFE2L3, FGF2, ABCA3, RNF165, PKDCC, and ZBTB46 in a sample comprising a cancer cell from the patient, normalized against the expression levels of all RNA transcripts in the sample or a reference set of mRNA expression levels, wherein the mRNA expression levels of MGAT3, STAR, VCAM1, RAB42, NFE2L3, FGF2, ABCA3, RNF165, PKDCC, and ZBTB46 are indicative of NF-kB activity, thereby evaluating the prognosis of the head and neck cancer patient.

21. The method of claim 20, wherein the mRNA expression of MGAT3, STAR, VCAM1, RAB42, NFE2L3, FGF2, ABCA3, RNF165, PKDCC, ZBTB46, IL27RA, KREMEN2, ARNT2, MMP19, PARM1, VRK2, COL22A1, BIRC3, SIM2, MEGF10, MAP3K14, C9orf172, C11orf92, CDH23, and C8orf42 are measured.

22. The method of claim 20, wherein the mRNA expression of MGAT3, STAR, VCAM1, RAB42, NFE2L3, FGF2, ABCA3, RNF165, PKDCC, ZBTB46, IL27RA, KREMEN2, ARNT2, MMP19, PARM1, VRK2, COL22A1, BIRC3, SIM2, MEGF10, MAP3K14, C9orf172, C11orf92, CDH23, C8orf42, ERO1LB, TMEM150C, SV2B, FAM105B, C9orf98, CYP27A1, LIFR, RTN4RL1, LOC283174, MCF2L, NEDD1, LOC100272146, TLR6, GALNT11, CDRT4, NT5DC1, TRAF2, FAM65C, ITGAM, ZNF488, RELB, VSTM2L, LGI2, FAM164A, and NOXO1 are measured.

23. The method of claim 20, wherein the head and neck cancer is an oropharyngeal squamous cell carcinoma (OPSCC), a nasopharyngeal squamous cell carcinoma, a squamous cell carcinomas of the nasal cavity or paranasal sinuses, a squamous cell carcinoma of the oral cavity, or a squamous cell carcinoma of the hypopharynx.

24. (canceled)

25. The method of claim 1, further comprising detecting defects in a biomarker for ESR1 (estrogen receptor).

26. (canceled)

27. (canceled)

28. An isolated and purified probe for specifically detecting defects in (a) nucleic acids encoding CYLD mutation N300S or D618A, or (b) their expression products.

29. The probe of claim 28, wherein the probe for detecting defects in nucleic acids is a PCR primer or probe.

30. The probe of claim 29, wherein the PCR primer is SEQ ID NO. 1, SEQ ID NO. 2, SEQ ID NO. 3, or SEQ ID NO. 4.

31. The probe of claim 28, where in the probe specifically detects SEQ ID NO. 6 or SEQ ID NO. 8.


SEQUENCE LISTING (8 sequences)

SEQ ID NO: 1 - DNA, 27 bp, artificial sequence
  Synthetic sequence
acatcagtgatatcatcccagctttat

SEQ ID NO: 2 - DNA, 27 bp, artificial sequence
  Synthetic sequence
gcaatagaattgtactttcaacacacg

SEQ ID NO: 3 - DNA, 39 bp, artificial sequence
  Synthetic sequence
gggtctaagtaacacagtggccagaacagaactaaaagc

SEQ ID NO: 4 - DNA, 39 bp, artificial sequence
  Synthetic sequence
gcttttagttctgttctggccactgtgttacttagaccc

SEQ ID NO: 5 - DNA, 21 bp, Homo sapiens
  exon2 (1)..(21)
ttgcacatcaatgatatcatc

SEQ ID NO: 6 - DNA, 21 bp, Homo sapien
ttgcagatcagtgatatcatc

SEQ ID NO: 7 - DNA, 21 bp, homo sapien
tctgttctggacactgtgtta

SEQ ID NO: 8 - DNA, 21 bp, homo sapien
tctggtctggccactgtgtta